(19) United States
(12) Patent Application Publication
(10) Pub. No.: US 2007/0254872 A1
(43) Pub. Date: Nov. 1, 2007

(54) Antibacterial Agents

(75) Inventors: William Henry Miller, Collegeville, PA (US); Meagan B. Rouse, Collegeville, PA (US); Mark Andrew Seefeld, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford, Middlesex (GB)

(21) Appl. No.: 11/570,441

(22) PCT Filed: Jul. 8, 2005
(86) PCT No.: PCT/US05/24221

§ 371 Date: Dec. 12, 2006

Related U.S. Application Data
(60) Provisional application No. 60/586,446, filed on Jul. 8, 2004.

(51) Int. Cl. A61K 31/5415; A61K 31/5383; A61P 31/04; C07D 265/36; C07D 279/16

(52) U.S. Cl. 514/224.2; 514/230.5; 544/105; 544/51

(57) ABSTRACT

Naphthalene, quinoline, quinoxaline and naphthyridine derivatives useful in the treatment of bacterial infections in mammals, particularly humans, are disclosed herein.

### FIELD OF THE INVENTION

This invention relates to novel compounds, compositions containing them and their use as antibacterials.

### BACKGROUND OF THE INVENTION

The emergence of pathogens resistant to known antibiotic therapy is becoming a serious global healthcare problem (Chu, et al., (1996) J. Med. Chem., 39: 3853-3874). Thus, there is a need to discover new broad spectrum antibiotics useful in combating multidrug-resistant organisms. Importantly, it has now been discovered that certain compounds have antibacterial activity, and, therefore, may be useful for the treatment of bacterial infections in mammals, particularly in humans.

### SUMMARY OF THE INVENTION

This invention comprises compounds of the formula (I), as described hereinafter, which are useful in the treatment of bacterial infections. This invention is also a pharmaceutical composition comprising a compound according to formula (I) and a pharmaceutically acceptable carrier. This invention is also processes for the preparation of compounds of formula (I), as well as processes for the preparation of intermediates useful in the synthesis of compounds of formula (I). This invention is also a method of treating bacterial infections in mammals, particularly in humans.

### DETAILED DESCRIPTION OF THE INVENTION

This invention provides a compound of formula (I) or a pharmaceutically acceptable salt, solvate or derivative thereof:

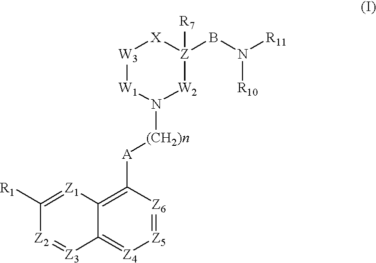

wherein:

Z₁, Z₃, and Z₄ are independently N or CR^{1a};

Z₂, Z₅ and Z₆ are each CR^{1a};

R₁ and R^{1a} are independently at each occurrence hydrogen; cyano; halogen; hydroxy; (C₁₋₆)alkoxy unsubstituted or substituted by (C₁₋₆)alkoxy, hydroxy, amino, piperidyl, guanidino or amidino any of which is unsubstituted or N-substituted by one or two (C₁₋₆)alkyl, acyl, (C₁₋₆)alkylsulphonyl, CONH₂, hydroxy, (C₁₋₆)alkylthio, heterocyclylthio, heterocyclyloxy, arylthio, aryloxy, acylthio, acyloxy or (C₁₋₆)alkylsulphonyloxy; (C₁₋₆)alkyl; (C₁₋₆)alkylthio; trifluoromethyl; trifluoromethoxy; nitro; azido; acyl; acyloxy; acylthio; (C₁₋₆)alkylsulphonyl; (C₁₋₆)alkylsulphoxide; arylsulphonyl; arylsulphoxide; or an amino, piperidyl, guanidino or amidino group unsubstituted or N-substituted by one or two (C₁₋₆)alkyl, acyl or (C₁₋₆)alkylsulphonyl groups; or R₁ and R^{1a} of Z₂ together form ethylenedioxy;

A is CR₂R₃ or NR^{1b}(C═O);

R₂ is hydrogen; halogen; hydroxy; acyloxy; or (C₁₋₆)alkoxy;

R₃ is hydrogen;

n is independently at each occurrence 0, 1, or 2;

R^{1b} is hydrogen; trifluoromethyl; (C₁₋₆)alkyl; (C₂₋₆)alkenyl; (C₁₋₆)alkoxycarbonyl; (C₁₋₆)alkylcarbonyl; (C₂₋₆)alkenyloxycarbonyl; aryl; aralkyl; (C₃₋₈)cycloalkyl; heterocyclyl; or heterocyclylalkyl;

W₁, W₂ and W₃ are CR₄R₅;

R₄, R₈, and R₉ are independently at each occurrence hydrogen; thiol; (C₁₋₆)alkylthio; halogen; trifluoromethyl; azido; (C₁₋₆)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aryl; aralkyl; aryl; heterocyclyl; heterocyclylalkyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted with hydrogen; (C₁₋₆)alkyl; (C₂₋₆)alkenyl; or aralkyl;

X is O, CR₄R₅, or NR₆;

R₅ is independently at each occurrence hydrogen or (C₁₋₆)alkyl;

R₆ is hydrogen; (C₁₋₆)alkyl; or together with R₁₀ forms Y;

Y is CR₄R₅CH₂; CH₂CR₄R₅; (C═O); CR₄R₅; CR₄R₅(C═O); or (C═O)CR₄R₅;

R₇ is hydrogen; halogen; hydroxy; or (C₁₋₆)alkyl;

Z is carbon;

B is CR₈R₉ or (C═O);

R₁₀ is hydrogen; (C₁₋₆)alkyl or together with R₆ forms Y;

R₁₁ is UR₁₂;

U is CR₄R₅; C(═O); or S(O)ₙ;

R₁₂ is a substituted or unsubstituted bicyclic carbocyclic or heterocyclic ring system (A):

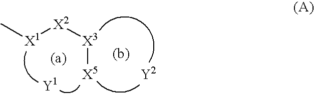

containing up to four heteroatoms in each ring in which at least one of rings (a) and (b) is aromatic;

X¹ is C or N when part of an aromatic ring or CR₁₃ when part of a non aromatic ring;

X² is N, NR₁₄, O, S(O)ₙ, CO or CR₁₃ when part of an aromatic or non-aromatic ring or may in addition be CR₁₅R₁₆ when part of a non aromatic ring;

X³ and X⁵ are independently N or C;

Y¹ is a 0 to 4 atom linker group each atom of which is independently selected from N, NR₁₄, O, S(O)ₙ, CO and CR₁₃ when part of an aromatic or non-aromatic ring or may additionally be CR₁₅R₁₆ when part of a non aromatic ring,

Y² is a 2 to 6 atom linker group, each atom of Y² being independently selected from N, NR₁₄, O, S(O)ₙ, CO and CR₁₃ when part of an aromatic or non-aromatic ring or may additionally be CR₁₅R₁₆ when part of a non aromatic ring;

R₁₃, R₁₅ and R₁₆ are at each occurrence independently selected from: hydrogen; (C₁₋₄)alkylthio; halo; (C₁₋₄)alkyl; (C₂₋₄)alkenyl; hydroxy; hydroxy(C₁₋₄)alkyl; mercapto(C₁₋₄)alkyl; (C₁₋₄)alkoxy; trifluoromethoxy; nitro; cyano; carboxy; amino or aminocarbonyl unsubstituted or substituted by (C₁₋₄)alkyl;

R₁₄ is at each occurrence independently hydrogen; trifluoromethyl; (C₁₋₄)alkyl unsubstituted or substituted by hydroxy, carboxy, (C₁₋₄)alkoxy, (C₁₋₆)alkylthio, halo or trifluoromethyl; (C₂₋₄)alkenyl; or aminocarbonyl wherein the amino group is optionally substituted with (C₁₋₄)alkyl;

or a pharmaceutically acceptable salt or solvate thereof;

provided that when Z₁ and Z₃ are CR^{1a}; Z₄ is N; X is O or CR₄R₅; and A is CR₂R₃; then R₂ is not hydroxy.

In one aspect, this invention describes a compound of formula (I) wherein Z₁ and Z₄ are N and Z₃ is CR^{1a}.

In other aspects, this invention describes a compound of formula (I) wherein Z₁ and Z₃ are CR^{1a} and Z₄ is N.

In some embodiments, this invention describes a compound of formula (I) wherein R₁ is OCH₃.

In some embodiments, this invention describes a compound of formula (I) wherein R^{1a} is at each occurrence independently hydrogen; halogen; or cyano.

In certain embodiments, this invention describes compounds of formula (I) wherein Z₁ and Z₄ are N and Z₃ is CR^{1a}; R^{1a} of Z₂, Z₃ and Z₅ are each hydrogen; R^{1a} of Z₆ is fluorine or cyano; and R₁ is OCH₃.

In certain aspects, this invention describes a compound of formula (I) wherein A is CH₂; and n of (CH₂)ₙ is 1.

In some aspects, this invention describes a compound of formula (I) wherein X is O.

In some embodiments, this invention describes a compound of formula (I) wherein X is CR₄R₅.

In some embodiments, this invention describes a compound of formula (I) wherein X is NR₆.

In some embodiments, this invention describes a compound of formula (I) wherein X is NR₆ and R₆ and R₇ together form Y.

In some embodiments, this invention describes a compound of formula (I) wherein X is NR₆ and R₆ and R₇ together form Y, and Y is CR₄R₅(C═O); (C═O); or (C═O)CR₄R₅.

In some embodiments, this invention describes a compound of formula (I) wherein X is NR₆ and R₆ and R₇ together form Y and Y is CH₂(C═O); (C═O); or (C═O)CH₂.

In some embodiments, this invention describes a compound of formula (I) wherein X is NR₆ and R₆ and R₇ together form Y, and Y is CR₄R₅(C═O); (C═O); or (C═O)CR₄R₅; and R₁₂ is 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl, 2,3-Dihydro-benzofuran-7-carbonitrile-5-yl; or [1,3]Oxathiolo[5,4-c]pyridin-6-yl.

In certain embodiments, this invention describes a compound of formula (I) wherein U is CH₂.

In some embodiments, this invention describes a compound of formula (I) wherein U is SO₂.

In some embodiments, this invention describes a compound of formula (I) wherein U is (C═O).

In some aspects, this invention describes a compound of formula (I) wherein R₁₂ is 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl; or [1,3]Oxathiolo[5,4-c]pyridin-6-yl.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is O; B is CH₂; and R₁₀ is hydrogen.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is O; B is CH₂; R₁₀ is hydrogen; and U is CH₂.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is O; B is CH₂; R₁₀ is hydrogen; and U is (C═O).

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is O; B is CH₂; R₁₀ is hydrogen; and U is SO₂.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano, A is CH₂; n of (CH₂)ₙ is 1; R₄ is independently at each occurrence selected from the group consisting of hydrogen; hydroxy or halogen; X is O; B is CH₂; R₁₀ is hydrogen; U is CH₂; and R₁₂ is: 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl; or [1,3]Oxathiolo[5,4-c]pyridin-6-yl.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is fluorine or cyano; A is CH₂; n of (CH₂)ₙ is 1; R₄ is independently at each occurrence selected from the group consisting of hydrogen; hydroxy or halogen; X is O; B is CH₂; R₁₀ is hydrogen; U is CH₂; and R₁₂ is: 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl; or [1,3]Oxathiolo[5,4-c]pyridin-6-yl.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is fluorine or cyano; A is CH₂; n of (CH₂)ₙ is 1; R₄ is independently at each occurrence selected from the group consisting of hydrogen; hydroxy or halogen; X is O; B is CH₂; R₇ is hydrogen; R₁₀ is hydrogen; U is CH₂; stereochemistry at Z is (S); and R₁₂ is: 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl; or [1,3]Oxathiolo[5,4-c]pyridin-6-yl.

In certain embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is NR^{1b}(C═O); n of (CH₂)ₙ is 0; R₄ is independently at each occurrence selected from the group consisting of hydrogen; hydroxy or halogen; X is O; B is CH₂; and R₁₀ is hydrogen.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is NR^{1b}(C═O); n of (CH₂)ₙ is 0; R₄ is independently at each occurrence selected from the group consisting of hydrogen; hydroxy or halogen; X is O; B is CH₂; R₁₀ is hydrogen; U is CH₂; and R₁₂ is 4H-Pyrido[3,2-b][1,4]thiazin-3-oxo-6-yl; 8-Cyano-2,3-dihydro-benzo[1,4]-dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl; or [1,3]Oxathiolo[5,4-c]pyridin-6-yl.

In some embodiments, this invention describes a compound of formula (I) wherein Z₁ and Z₄ are N; Z₃ is CR^{1a}; R₁ is OCH 3; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is CR₄R₅; R₇ is hydrogen; B is CH₂; and R₁₀ is hydrogen.

In some embodiments, this invention describes a compound of formula (I) wherein Z₁ and Z₄ are N; Z₃ is CR^{1a}; R₁ is OCH 3; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is CR₄R₅; R₇ is hydrogen; B is CH₂; R₁₀ is hydrogen; and R₁₂ is 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl; or [1,3]Oxathiolo[5,4-c]pyridin-6-yl.

In one aspect, this invention describes a compound of formula (I) wherein Z₁ and Z₄ are N; Z₃ is CR^{1a}; R^{1a} is OCH₃; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is NR₆; R₆ is hydrogen or (C₁₋₆)alkyl; B is CH₂; and R₁₀ is hydrogen.

In some embodiments, this invention describes a compound of formula (I) wherein Z₁ and Z₄ are N; Z₃ is CR^{1a}; R₁ is OCH₃; R^{1a} of Z₃, Z₄ and Z₅ is hydrogen; R^{1a} of Z₆ is hydrogen, fluorine or cyano; A is CH₂; n of (CH₂)ₙ is 1; R₄ is independently at each occurrence selected from the group consisting of hydrogen; hydroxy or halogen; X is NR₆; R₆ is hydrogen or (C₁₋₆)alkyl; B is CH₂; R₁₀ is hydrogen; U is CH₂ and R₁₂ is 4H-Pyrido[3,2-b][1,4]thiazin-3-oxo-6-yl; 8-Cyano-2,3-dihydro-benzo[1,4]dioxin-6-yl; 5-Cyano-2,3-dihydro-benzo[1,4]dioxin-7-yl; 4H-Pyrido[3,2-b][1,4]oxazin-3-oxo-6-yl; 8-Fluoro-4H-[1,4]-benzoxazin-3-oxo-6-yl; 4H-Benzo[1,4]thiazin-3-oxo-6-yl; 7-Chloro-4H-pyrido[3,2-b]oxazin-3-oxo-6-yl; 2,3-Dihydro-benzofuran-7-carbonitrile-5-yl or [1,3]Oxathiolo[5,4-c]pyridin-6-yl.

In certain aspects, this invention describes a process for the preparation of intermediates of formula (IV) useful in the preparation of compounds of formula (I), which process comprises:

(a) reacting a compound or formula (II) with a compound of formula (III) to give a useful intermediate having formula (IV): 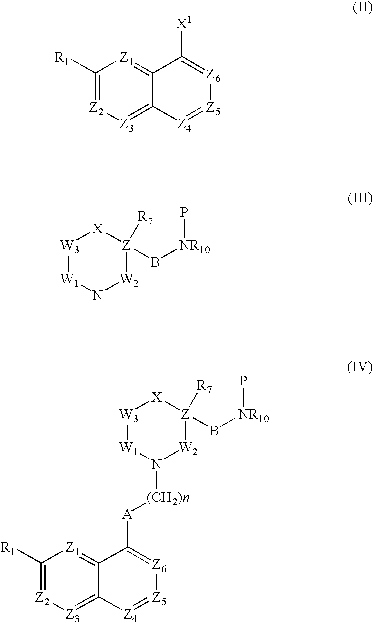
wherein:
Z₁, R₁, R₂, R₃, Z₂, Z₃, Z₄, Z₅, Z₆, n, W₁, W₂ W₃, X, Z, R₇, B and R₁₀ are as defined in claim 1; and
X′ is CH═CH₂ or A-(CH₂)ₙ-L;
A is CR₂R₃;
L is a leaving group; and
P is hydrogen or an amine protecting group.

In some embodiments, this invention describes a process for the preparation of a compound of claim 1, which process comprises:

(a) reacting a compound of formula (II) with a compound of formula (III) to give a compound of formula (IV);

(b) reacting the compound of formula (IV) with a compound of formula (V);

(c) removing P (where P is not hydrogen) to give a compound of formula (I);

(d) optionally converting to a pharmaceutically acceptable salt or solvate, thereof;

or

(a) reacting a compound of formula (II) with a compound of formula (III) to give a compound of formula (IV);

(b) removing P (where P is not hydrogen); and

(c) reacting the product of step (b) with a compound of formula (V) to give a compound of formula (I);

(d) optionally converting to a pharmaceutically acceptable salt or solvate, thereof;

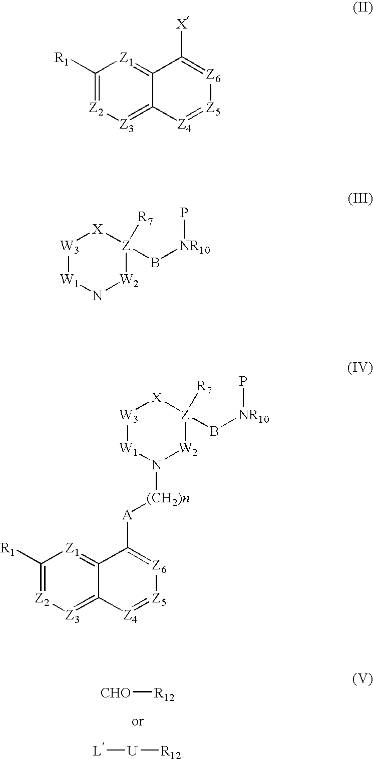

wherein:

Z₁, R₁, R₂, R₃, Z₂, Z₃, Z₄, Z₅, Z₆, n, W₁, W₂, W₃, X, Z, R₇, B, R₁₀, R₁₂ and U are as defined in claim 1; and

X′ is CH═CH₂ or A-(CH₂)ₙ-L;

A is CR₂R₃;

L and L′ are leaving groups; and

P is hydrogen or an amine protecting group.

In certain aspects, this invention describes a compound of formula (I) wherein said compound is 6-({[(1-{2-[6-(methyloxy)-1,5-naphthyridin-4-yl]ethyl}-3-piperidinyl)methyl]amino}methyl)-2H-pyrido[3,2-b][1,4]thiazin-3(4H)-one; N-[(1-{2-[6-(methyloxy)-1,5-naphthyridin-4-yl]ethyl}-3-piperidinyl)methyl]-3-oxo-3,4-dihydro-2H-1,4-benzothiazine-6-sulfonamide; N-[(1-{2-[6-(methyloxy)-1,5-naphthyridin-4-yl]ethyl}-3-piperidinyl)methyl]-3-oxo-3,4-dihydro-2H-pyrido[3,2-b][1,4]thiazine-6-carboxamide; 6-({[(4-{2-[6-(methyloxy)-1,5-naphthyridin-4-yl]ethyl}-2-morpholinyl)methyl]amino}methyl)-2H-pyrido[3,2-b][1,4]thiazin-3(4H)-one; N-[(4-{2-[6-(methyloxy)-1,5-naphthyridin-4-yl]ethyl}-2-morpholinyl)methyl]-3-oxo-3,4-dihydro-2H-pyrido[3,2-b][1,4]thiazine-6-carboxamide; N-[(4-{2-[6-(methyloxy)-1,5-naphthyridin-4-yl]ethyl}-2-morpholinyl)methyl]-3-oxo-3,4-dihydro-2H-1,4-benzothiazine-6-sulfonamide; 6-({[((2R)-4-{2-[6-(methyloxy)-1,5-naphthyridin-4-yl]ethyl}-2-morpholinyl)methyl]amino}methyl)-2H-pyrido[3,2-b][1,4]thiazin-3(4H)-one; 6-({[((2S)-4-{2-[6-(methyloxy)-1,5-naphthyridin-4-yl]ethyl}-2-morpholinyl)methyl]amino}methyl)-2H-pyrido[3,2-b][1,4]thiazin-3(4H)-one; 6-({[((2R)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one; 6-({[((2S)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one; N-methyl-4-{2[6-(methyloxy)-1,5-naphthyridin-4-yl]ethyl}-N-[(3-oxo-3,4-dihydro-2H-pyrido[3,2-b][1,4]thiazin-6-yl)methyl]-2-morpholinecarboxamide; 6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]thiazin-3(4H)-one; 6-({[(4-{2-[3-fluoro-6-(methyloxy)-1,5-naphthyridin-4-yl]ethyl}-2-piperazinyl)methyl]amino}methyl)-2H-pyrido[3,2-b][1,4]thiazin-3(4H)-one; 6-{[7-{2-[3-fluoro-6-(methyloxy)-1,5-naphthyridin-4-yl]ethyl}-3-oxohexahydroimidazo[1,5-a]pyrazin-2(3H)-yl]methyl}-2H-pyrido[3,2-b][1,4]thiazin-3(4H)-one; 6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]oxazin-3(4H)-one; 6-(methyloxy)-4-{2-[(2S)-2-({[(3-oxo-3,4-dihydro-2H-pyrido[3,2-b][1,4]oxazin-6-yl)methyl]amino}methyl)-4-morpholinyl]ethyl}-1,5-naphthyridine-3-carbonitrile; 6-(methyloxy)-4-{2-[(2S)-2-({[(3-oxo-3,4-dihydro-2H-pyrido[3,2-b][1,4]thiazin-6-yl)methyl]amino}methyl)-4-morpholinyl]ethyl}-1,5-naphthyridine-3-carbonitrile; 6-({[((2S)-4-{2-[3-fluoro-6-(methyloxy)-4-quinolinyl]ethyl}-2-morpholinyl)methyl]amino}methyl)-2H-pyrido[3,2-b][1,4]thiazin-3(4H)-one; 6-({[((2S)-4-{2-[3-fluoro-6-(methyloxy)-4-quinolinyl]ethyl}-2-morpholinyl)methyl]amino}methyl)-2H-pyrido[3,2-b][1,4]oxazin-3(4H)-one; 8-fluoro-6-({[((2S)-4-{2-[3-fluoro-6-(methyloxy)-1,5-naphthyridin-4-yl]ethyl}-2-morpholinyl)methyl]amino}methyl)-2H-1,4-benzoxazin-3(4H)-one; 7-chloro-6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]oxazin-3(4H)-one; [((2S)-4-{2-[3-fluoro-6-(methyloxy)-1,5-naphthyridin-4-yl]ethyl}-2-morpholinyl)methyl]([1,3]oxathiolo[5,4-c]pyridin-6-ylmethyl)amine; 7-({[((2S)-4-{2-[3-fluoro-6-(methyloxy)-1,5-naphthyridin-4-yl]ethyl}-2-morpholinyl)methyl]amino}methyl)-2,3-dihydro-1,4-benzodioxin-5-carbonitrile; 5-({[((2S)-4-{2-[3-fluoro-6-(methyloxy)-1,5-naphthyridin-4-yl]ethyl}-2-morpholinyl)methyl]amino}methyl)-2,3-dihydro-1-benzofuran-7-carbonitrile; 6-({[((2S)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one; 6-({[((2S)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one; 6-({[((2R)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one; 6-({[((2R)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one; 6-[(8-{2-[3-fluoro-6-(methyloxy)-1,5-naphthyridin-4-yl]ethyl}-4-oxooctahydro-2H-pyrazino[1,2-a]pyrazin-2-yl)methyl]-2H-pyrido[3,2-b][1,4]thiazin-3(4H)-one; 6-[(8-{2-[3-fluoro-6-(methyloxy)-1,5-naphthyridin-4-yl]ethyl}-3-oxooctahydro-2H-pyrazino[1,2-a]pyrazin-2-yl)methyl]-2H-pyrido[3,2-b][1,4]thiazin-3(4H)-one; 6-({[(4-{2-[6-(methyloxy)-1,5-naphthyridin-4-yl]ethyl}-2-piperazinyl)methyl]amino}methyl)-2H-pyrido[3,2-b][1,4]thiazin-3(4H)-one; (2S)—N-[6-(methyloxy)-1,5-naphthyridin-4-yl]-2-({[(3-oxo-3,4-dihydro-2H-pyrido[3,2-b][1,4]thiazin-6-yl)methyl]amino}methyl)-4-morpholinecarboxamide; or a pharmaceutically acceptable salt or solvate thereof.

In certain embodiments, this invention describes a pharmaceutical composition comprising a compound of formula I or any one of the embodiments described herein, and a pharmaceutically acceptable carrier.

In some embodiments, this invention describes a method of treating bacterial infections which comprises administering to a mammal in need thereof an effective amount of a compound of formula or any of its embodiments described herein.

In some embodiments, this invention describes compounds of formula I wherein the (a) and (b) rings of R₁₁ are both aromatic as demonstrated by the following non-limiting examples: 1H-pyrrolo[2,3-b]-pyridin-2-yl, 1H-pyrrolo[3,2-b]-pyridin-2-yl, 3H-imidazo[4,5-b]-pyrid-2-yl, 3H-quinazolin-4-one-2-yl, benzimidazol-2-yl, benzo[1,2,3]-thiadiazol-5-yl, benzo[1,2,5]-oxadiazol-5-yl, benzofur-2-yl, benzothiazol-2-yl, benzo[b]thiophen-2-yl, benzoxazol-2-yl, chromen-4-one-3-yl, imidazo[1,2-a]pyridin-2-yl, imidazo-[1,2-a]-pyrimidin-2-yl, indol-2-yl, indol-6-yl, isoquinolin-3-yl, [1,8]-naphthyridine-3-yl, oxazolo[4,5-b]-pyridin-2-yl, quinolin-2-yl, quinolin-3-yl, quinoxalin-2-yl, indan-2-yl, naphthalen-2-yl, 1,3-dioxo-isoindol-2-yl, benzimidazol-2-yl, benzothiophen-2-yl, 1H-benzotriazol-5-yl, 1H-indol-5-yl, 3H-benzooxazol-2-one-6-yl, 3H-benzooxazol-2-thione-6-yl, 3H-benzothiazol-2-one-5-yl, 3H-quinazolin-4-one-2-yl, 3H-quinazolin-4-one-6-yl, 4-oxo-4H-pyrido[1,2-a]pyrimidin-3-yl, benzo[1,2,3]thiadiazol-6-yl, benzo[1,2,5]thiadiazol-5-yl, benzo[1,4]oxazin-2-one-3-yl, benzothiazol-5-yl, benzothiazol-6-yl, cinnolin-3-yl, imidazo[1,2-a]pyridazin-2-yl, imidazo[1,2-b]pyridazin-2-yl, pyrazolo[1,5-a]pyrazin-2-yl, pyrazolo[1,5-a]pyridin-2-yl, pyrazolo[1,5-a]pyrimidin-6-yl, pyrazolo[5,1-c][1,2,4]triazin-3-yl, pyrido[1,2-a]pyrimidin-4-one-2-yl, pyrido[1,2-a]pyrimidin-4-one-3-yl, quinazolin-2-yl, quinoxalin-6-yl, thiazolo[3,2-a]pyrimidin-5-one-7-yl, thiazolo[5,4-b]pyridin-2-yl, thieno[3,2-b]pyridin-6-yl, thiazolo[5,4-b]pyridin-6-yl, 4-oxo-4H-pyrido[1,2-a]pyrimidin-2-yl, 1-oxo-1,2-dihydro-isoquinolin-3-yl, thiazolo[4,5-b]pyridin-5-yl, [1,2,3]thiadiazolo[5,4-b]pyridin-6-yl, 2H-isoquinolin-1-one-3-yl.

In yet other embodiments, R₁₁ is defined by a non-aromatic (a) ring and aromatic (b) ring as illustrated by the following non-limiting examples:_(2S)-2,3-dihydro-1H-indol-2-yl, (2S)-2,3-dihydro-benzo[1,4]dioxine-2-yl, 3-(R,S)-3,4-dihydro-2H-benzo[1,4]thiazin-3-yl, 3-(R)-2,3-dihydro-[1,4]dioxino[2,3-b]pyridin-3-yl, 3-(S)-2,3-dihydro-[1,4]dioxino[2,3-b]pyridin-3-yl, 2,3-dihydro-benzo[1,4]dioxan-2-yl, 3-substituted-3H-quinazolin-4-one-2-yl, 2,3-dihydro-benzo[1,4]dioxan-2-yl, 1-oxo-1,3,4,5-tetrahydrobenzo[c]azepin-2-yl. In still other embodiments, R₁₁ is defined by an aromatic (a) ring and a non aromatic (b) ring as illustrated by the following non-limiting examples: 1,1,3-trioxo-1,2,3,4-tetrahydro-1 β-benzo[1,4]thiazin-6-yl, benzo[1,3]dioxol-5-yl, 2,3-dihydro-benzo[1,4]dioxin-6-yl, 2-oxo-2,3-dihydro-benzooxazol-6-yl, 4H-benzo[1,4]oxazin-3-one-6-yl(3-oxo-3,4-dihydro-2H-benzo[1,4]oxazin-6-yl), 4H-benzo[1,4]thiazin-3-one-6-yl(3-oxo-3,4-dihydro-2H-benzo[1,4]thiazin-6-yl), 4H-benzo[1,4]oxazin-3-one-7-yl, 4-oxo-2,3,4,5-tetrahydro-benzo[b][1,4]thiazepine-7-yl, 5-oxo-2,3-dihydro-5H-thiazolo[3,2-a]pyrimidin-6-yl, benzo[1,3]dioxol-5-yl, 2-oxo-2,3-dihydro-1H-pyrido[2,3-b][1,4]thiazin-7-yl, 2-oxo-2,3-dihydro-1H-pyrido[3,4-b][1,4]thiazin-7-yl, 3-oxo-3,4-dihydro-2H-pyrido[3,2-b][1,4]thiazin-6-yl, 2,3-dihydro-[1,4]dioxino[2,3-b]pyridin-6-yl, 2,3-dihydro-[1,4]dioxino[2,3-c]pyridin-7-yl, 2,3-dihydro-[1,4]dioxino[2,3-b]pyridin-7-yl, 6,7-dihydro-[1,4]dioxino[2,3-d]pyrimidin-2-yl, 3-oxo-3,4-dihydro-2H-pyrido[3,2-b][1,4]oxazin-6-yl, 2-oxo-2,3-dihydro-1H-pyrido[3,4-b][1,4]oxazin-7-yl, 2-oxo-2,3-dihydro-1H-pyrido[2,3-b][1,4]oxazin-7-yl, 6-oxo-6,7-dihydro-5H-8-thia-1,2,5-triaza-naphthalen-3-yl, 3,4-dihydro-2H-benzo[1,4]oxazin-6-yl, 3-substituted-3H-benzooxazol-2-one-6-yl, 3-substituted-3H-benzooxazole-2-thione-6-yl, 3-substituted-3H-benzothiazol-2-one-6-yl, 2,3-dihydro-1H-pyrido[2,3-b][1,4]thiazin-7-yl, 3,4-dihydro-2H-benzo[1,4]thiazin-6-yl, 3,4-dihydro-1H-quinolin-2-one-7-yl, 3,4-dihydro-1H-quinoxalin-2-one-7-yl, 6,7-dihydro-4H-pyrazolo[1,5-a]pyrimidin-5-one-2-yl, 5,6,7,8-tetrahydro-[1,8]naphthyridin-2-yl, 2-oxo-3,4-dihydro-1H-[1,8]naphthyridin-6-yl, 3,4-dihydro-2H-pyrido[3,2-b][1,4]thiazin-6-yl.

Unless otherwise defined, the term “alkyl” when used alone or when forming part of other groups (such as the ‘alkoxy’ group) includes substituted or unsubstituted, straight or branched chain alkyl groups containing the specified range of carbon atoms. For example, the term “(C₁₋₆)alkyl” include methyl, ethyl, propyl, butyl, iso-propyl, sec-butyl, tert-butyl, iso-pentyl, and the like.

The term “alkenyl” means a substituted or unsubstituted alkyl group of the specified range of carbon atoms, wherein one carbon-carbon single bond is replaced by a carbon-carbon double bond. For example, the term “(C₂₋₆)alkenyl” include ethylene, 1-propene, 2-propene, 1-butene, 2-butene, and isobutene, and the like. Both cis and trans isomers are included.

The term “cycloalkyl” refers to substituted or unsubstituted carbocyclic system of the specified range of carbon atoms, which may contain up to two unsaturated carbon-carbon bonds. For example, the term “(C₃₋₇)cycloalkyl” include cyclopropyl, cyclobutyl, cyclopentyl, cyclopentenyl, cyclohexyl, cyclohexenyl, and cycloheptyl.

The term “alkoxy” refers to an O-alkyl radical where the alkyl group contains the specified range of carbon atoms and is as defined herein.

The term “acyl” refers to a C(═O)alkyl or a C(═O)aryl radical. In some embodiments, the alkyl group contains 13 or less carbons; in some embodiments 10 or less carbon atoms; in some embodiments 6 or less carbon atoms; and is as otherwise defined. Aryl is as defined herein.

The term “alkylcarbonyl” refers to a (C₁₋₆)alkyl(C═O)(C₁₋₆)alkyl group wherein alkyl is as otherwise defined herein.

The term “alkylsulphonyl” refers to a SO₂alkyl radical wherein the alkyl group contains the specified range of carbon atoms and is as defined herein.

The term “alkylthio” refers to a Salkyl wherein the alkyl group contains the specified range of carbon atoms and is as defined herein.

The term “aminosulphonyl” refers to a SO₂N(alkyl)₂ radical wherein the alkyl groups are independent from each other and as otherwise defined.

The term “aminocarbonyl” refers to a carboxamide radical wherein the nitrogen of the amide is substituted as defined.

The term “heterocyclylthio” refers to a S-heterocyclyl radical wherein the heterocyclyl moiety is as defined herein.

The term “heterocyclyloxy” refers to an O-heterocyclyl radical wherein heterocyclyl is as defined herein.

The term “arylthio” refers to an S-aryl radical wherein aryl is as defined herein.

The term “aryloxy” refers to an O-aryl radical wherein aryl is as defined herein.

The term “acylthio” refers to a S-acyl radical wherein acyl is as defined herein.

The term “acyloxy” refers to an O-acyl radical wherein acyl is as defined herein.

The term “alkoxycarbonyl” refers to a CO₂alkyl radical wherein the alkyl group contains the specified range of carbon atoms and is as defined herein.

The term “alkenyloxycarbonyl” refers to a CO₂alkyl radical wherein the alkenyl group contains the specified range of carbon atoms and is as defined herein.

The term “alkylsulphonyloxy” refers to an O—SO₂alkyl radical wherein the alkyl group contains the specified range of carbon atoms and is as defined herein.

The term “arylsulphonyl” refers to a SO₂aryl radical wherein aryl is as herein defined.

The term “arylsulphoxide” refers to a SOaryl radical wherein aryl is as defined herein.

Unless otherwise defined, suitable substituents for any alkyl, alkoxy, alkenyl, and cycloalkyl groups includes up to three substituents selected from the group consisting of hydroxy, halogen, nitro, cyano, carboxy, amino, amidino, sulphonamido, unsubstituted (C₁₋₃)alkoxy, trifluoromethyl, and acyloxy.

Halo or halogen includes fluoro, chloro, bromo and iodo.

The term “haloalkyl” refers to an alkyl radical containing the specified range of carbon atoms and is as otherwise defined herein, which is further substituted with 1-3 halogen atoms.

The term “haloalkoxy” refers to an alkoxy radical of the specified range and as defined herein, which is further substituted with 1-3 halogen atoms.

The term “hydroxyalkyl” refers to an alkyl group as defined herein, further substituted with a hydroxy group.

Unless otherwise defined, the term “heterocyclic” or “heterocyclyl” as used herein includes optionally substituted aromatic and non-aromatic, single and fused, mono- or bicyclic rings suitably containing up to four hetero-atoms in each ring selected from oxygen, nitrogen and sulphur, which rings may be unsubstituted or C-substituted by, for example, up to three groups selected from (C₁₋₄)alkylthio; halo; (C₁₋₄)haloalkoxy; (C₁₋₄)haloalkyl; (C₁₋₄)alkyl; (C₂₋₄)alkenyl; hydroxy; hydroxy, (C₁₋₄)alkyl; (C₁₋₄)thioalkyl; (C₁₋₄)alkoxy; nitro; cyano, carboxy; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl.

Each heterocyclic ring suitably has from 3 to 7, preferably 5 or 6, ring atoms. A fused heterocyclic ring system may include carbocyclic rings and need include only one heterocyclic ring.

Compounds within the invention containing a heterocyclyl group may occur in two or more tautometric forms depending on the nature of the heterocyclyl group; all such tautomeric forms are included within the scope of the invention.

Where an amino group forms part of a single or fused non-aromatic heterocyclic ring as defined above suitable optional substituents in such substituted amino groups include hydrogen; trifluoromethyl; (C₁₋₄)alkyl optionally substituted by hydroxy, (C₁₋₄)alkoxy, (C₁₋₄)alkylthio, halo or trifluoromethyl; and (C₂₋₄)alkenyl.

The term “heterocyclylalkyl” refers to a (C₁₋₆)alkyl radical which bears as a substituent a heterocyclyl group, wherein heterocyclyl and alkyl are as herein defined. The heterocyclyl group maybe joined to a primary, secondary or tertiary carbon of the (C₁₋₆)alkyl chain.

When used herein the term “aryl”, includes optionally substituted phenyl and naphthyl.

Aryl groups may be optionally substituted with up to five, preferably up to three, groups selected from (C₁₋₄)alkylthio; halo; (C₁₋₄)haloalkoxy; (C₁₋₄)haloalkyl; (C₁₋₄)alkyl; (C₂₋₄)alkenyl; hydroxy; (C₁₋₄)hydroxyalkyl; (C₁₋₄)alkylthio; (C₁₋₄)alkoxy; nitro; cyano; carboxy; amino or aminocarbonyl optionally substituted by (C₁₋₄)alkyl; (C₁₋₄)alkylsulphonyl; (C₂₋₄)alkenylsulphonyl.

The term “aralkyl” refers to a (C₁₋₆)alkyl radical which bears as a substituent an aryl group, wherein aryl and alkyl are as herein defined. The aryl group maybe joined to a primary, secondary or tertiary carbon of the (C₁₋₆)alkyl chain.

This invention also contemplates that some of its structural embodiments maybe present as a solvate. Solvates maybe produced from crystallization from a given solvent or mixture of solvents, inorganic or organic. Solvates may also produced upon contact or exposure to solvent vapors, such as water. This invention includes within its scope stoichiometric and non-stoichiometric solvates including hydrates as well as compounds containing variable amounts of water that may be produced by processes such as lyophilisation.

Furthermore, it will be understood that phrases such as “a compound of Formula I or a pharmaceutically acceptable salt, solvate or derivative thereof” are intended to encompass the compound of Formula I, a derivative of formula (I), a pharmaceutically acceptable salt of the compound of formula (I), a solvate of formula (I), or any pharmaceutically acceptable combination of these. Thus by way of non-limiting example used here for illustrative purpose, “a compound of Formula I or a pharmaceutically acceptable salt or solvate thereof” may include a pharmaceutically acceptable salt of a compound of formula (I) that is further present as a solvate.

Since the compounds of formula (I) are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

Pharmaceutically acceptable salts of the above-mentioned compounds of formula (I) include the free base form or their acid addition or quaternary ammonium salts, for example their salts with mineral acids e.g. hydrochloric, hydrobromic, sulphuric, nitric or phosphoric acids, or organic acids, e.g. acetic, fumaric, succinic, maleic, citric, benzoic, p-toluenesulphonic, methanesulphonic, naphthalenesulphonic acid or tartaric acids. Compounds of formula (I) may also be prepared as the N-oxide. Compounds of formula (I) having a free carboxy group may also be prepared as an in vivo hydrolysable ester. The invention extends to all such derivatives. One of skill in the art will recognize that where compounds of the invention contain multiple basic sites, a compound of the invention maybe present as a salt complexed with more than one equivalent of a corresponding acid or mixture of acids.

Pharmaceutically acceptable derivatives refers to compounds of formula (I) that have been covalently modified with a group that undergoes at least some in vivo cleavage to a compound of formula (I).

Examples of suitable pharmaceutically acceptable in vivo hydrolysable ester-forming groups include those forming esters which break down readily in the human body to leave the parent acid or its salt.

Suitable groups of this type include those of part formulae (i), (ii), (iii), (iv) and (v):

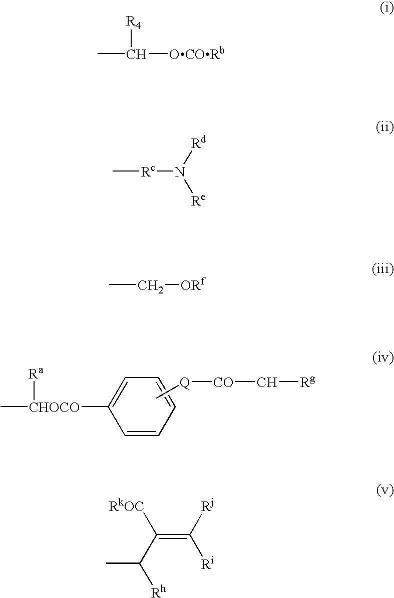

wherein R^{a} is hydrogen, (C₁₋₆) alkyl, (C₃₋₇) cycloalkyl, methyl, or phenyl, R^{b} is (C₁₋₆) alkyl, (C₁₋₆)alkoxy, phenyl, benzyl, (C₃₋₇)cycloalkyl, (C₃₋₇)cycloalkyloxy, (C₁₋₆)alkyl(C₃₋₇) cycloalkyl, 1-amino(C₁₋₆)alkyl, or 1-(C₁ alkyl)amino(C₁₋₆) alkyl; or R^{a} and R^{b} together form a 1,2-phenylene group optionally substituted by one or two methoxy groups; R^{c} represents (C₁₋₆)alkylene optionally substituted with a methyl or ethyl group and R^{d} and R^{e} independently represent (C₁₋₆)alkyl, R^{f} represents (C₁₋₆)alkyl; R^{g} represents hydrogen or phenyl optionally substituted by up to three groups selected from halogen, (C₁₋₆) alkyl, or (C₁₋₆) alkoxy; Q is oxygen or NH; R^{h} is hydrogen or

(C₁₋₆) alkyl; Rⁱ is hydrogen, (C₁₋₆) alkyl optionally substituted by halogen, (C₂₋₆) alkenyl, (C₁₋₆)alkoxycarbonyl, aryl or heteroaryl; or R^{h} and Rⁱ together form (C₁₋₆) alkylene; R^{j} represents hydrogen, (C₁₋₆) alkyl or (C₁₋₆)alkoxycarbonyl; and R^{k} represents (C₁₋₈)alkyl, (C₁₋₈)alkoxy, (C₁₋₆)alkoxy(C₁₋₆)alkoxy or aryl.

Examples of suitable in vivo hydrolysable ester groups include, for example, acyloxy(C₁₋₆)alkyl groups such as acetoxymethyl, pivaloyloxymethyl, acetoxyethyl, pivaloyloxyethyl, 1-(cyclohexylcarbonyloxy)prop-1-yl, and (1-aminoethyl)carbonyloxymethyl; (C₁₋₆)alkoxycarbonyloxy(C₁₋₆)alkyl groups, such as ethoxycarbonyloxymethyl, ethoxycarbonyloxyethyl and propoxycarbonyloxyethyl; di(C₁₋₆)alkylamino(C₁₋₆)alkyl especially di(C₁₋₄)alkylamino(C₁₋₄)alkyl groups such as dimethylaminomethyl, dimethylaminoethyl, diethylaminomethyl or diethylaminoethyl; 2-(C₁₋₆)alkoxycarbonyl)-2-(C₂₋₆)alkenyl groups such as 2-(isobutoxycarbonyl)pent-2-enyl and 2-(ethoxycarbonyl)but-2-enyl; lactone groups such as phthalidyl and dimethoxyphthalidyl.

A further suitable pharmaceutically acceptable in vivo hydrolysable ester-forming group is that of the formula:

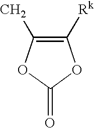

wherein R^{k} is hydrogen, C₁₋₆alkyl or phenyl.

R is preferably hydrogen.

Compounds of formula (I) may also be prepared as the corresponding N-oxides.

Certain of the compounds of formula (I) may exist in the form of optical isomers, e.g. diastereoisomers and mixtures of isomers in all ratios, e.g. racemic mixtures. The invention includes all such form, including pure isomeric forms. The different isomeric forms may be separated or resolved one from the other by conventional methods, or any given isomer may be obtained by conventional synthetic methods or by stereospecific or asymmetric syntheses.

One of skill in the readily appreciates that optimization for a given reaction may require some routine variation in reaction parameters such as reaction time, temperature, energy source, pressure, light, pressure, solvent or solvents used, co-reagents, catalysts, and the like.

Protective groups wherever found herein maybe designated by their specific formula or alternatively, maybe referred to generically by P or Pₙ (wherein n is an integer). It is to be appreciated that where generic descriptors are used, that such descriptors are at each occurrence independent from each other. Thus, a compound with more than one of the same generic descriptors (e.g. P) does not indicate that each P is the same protective group, they maybe the same or different, so long as the group is suitable to the chemistry being employed. Where protection or deprotection is generically referred to, one of ordinary skill in the art will understand this to mean that suitable conditions are employed that will allow for the removal of the protecting group to be removed while minimizing reaction at other positions of the molecule, unless otherwise indicated. Many protective groups and protective group strategies are known to those of skill in the art in maybe found in numerous references including, Greene, et al. “Protective Groups in Organic Synthesis” (Published by Wiley-Interscience), which is herein incorporated by reference in its entirety.

Leaving groups wherever found herein maybe designated by a specific chemical formula, or alternatively, maybe generically referred to as L, L′, Ln or L′n (wherein n is an integer). It is to be appreciated that where a generic descriptor is used, that such descriptors are at each occurrence independent from each other. Leaving groups can be single atoms such as Cl, Br, or I, or maybe a group such as OSO₂CH₃, OC(═O)CH₃, O(C═O)CF₃, OSO₂CF₃, and the like. Leaving groups may be formed during the course of a reaction and thus a compound containing a leaving group may not always be an isolated material but rather as a reactive intermediate. By way of non-limiting example, a carboxylic acid maybe reacted with a coupling reagent such as DCC, CDI, EDCI, isobutyl chloroformate, etc, and the corresponding reactive intermediate thus formed is further reacted with the nucleophilic coupling partner. In such cases, one of skill in the art appreciates that the activation step maybe performed before the introduction of the amine, or in some cases, even in the presence of the amine (depending upon the identity of the particular activating agent and carboxylic acid used). One skilled in the art readily ascertains that leaving groups generally refer to atoms or groups which can be eliminated, substituted or otherwise dissociate during the course of the reaction.

The antibacterial compounds according to the invention may be formulated for administration in any convenient way for use in human or veterinary medicine, by analogy with other antibacterials.

The pharmaceutical compositions of the invention include those in a form adapted for oral, topical or parenteral use and may be used for the treatment of bacterial infection in mammals including humans.

The composition may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

No toxicological effects are indicated when a compound of formula (I) or a pharmaceutically acceptable derivative thereof is administered in the above-mentioned dosage range.

The compound of formula (I) may be the sole therapeutic agent in the compositions of the invention or a combination with other antibacterials. If the other antibacterial is a β-lactam then a β-lactamase inhibitor may also be employed.

Compounds of formula (I) are active against a wide range of organisms including both Gram-negative and Gram-positive organisms.

The compounds of this invention may also be used in the manufacture of medicaments useful in treating bacterial infections in humans or other mammals.

All publications, including but not limited to patents and patent applications, cited in this specification are herein incorporated by reference (whether specifically stated to be so or not) as if each individual publication were specifically and individually indicated to be incorporated by reference herein as though fully set forth.

The following examples illustrate the preparation of certain compounds of formula (I) and the activity of certain compounds of formula (I) against various bacterial organisms.

The compounds of the present invention were prepared by the methods illustrated in Schemes I, II, III, IV, V, VI and VII. One of skill in the art readily appreciates that although the following schemes describe specific examples, they maybe more generally applied to produce additional embodiments of this invention. Furthermore, the examples set forth below are illustrative of the present invention and are not intended to limit, in any way, the scope of the present invention. 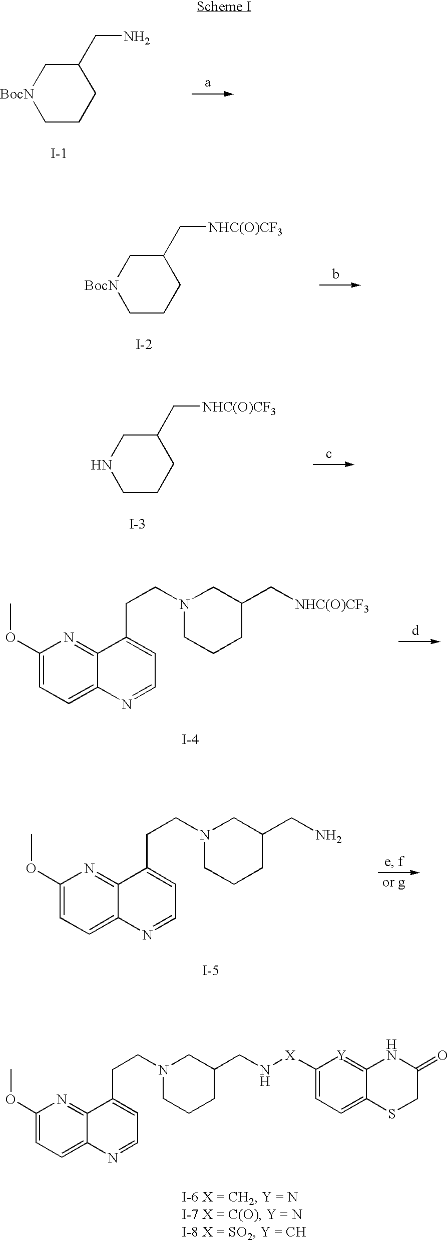
Reagents and conditions: (a) TFAA, DCM, pyr., 25° C. (b) TFA, 25° C. (c) 8-ethenyl-2-(methyloxy)-1,5-naphthyridine, DMF, 90° C. (d) K₂CO₃, MeOH—H₂O, 25° C. (e) 3-oxo-3,4-dihydro-2H-pyrido[3,2-b][1,4]thiazine-6-carbaldehyde, Na₂SO₄, DCM-EtOH; then NaBH₄, 25° C. (f) 3-oxo-3,4-dihydro-2H-pyrido[3,2-b][1,4]thiazine-6-carboxylic acid, 1-(3-Dimethylaminopropyl)-3-ethylcarbodimide, DCM-DMF, 25° C. (g) DIPEA, 3-oxo-3,4-dihydro-2H-pyrido[3,2-b][1,4]thiazine-6-sulfonyl chloride, DCM, 25° C. Commercial Boc-piperidinecarboxylate (I-1) was protected as the trifluoroacetamide (I-2). The Boc group was then removed and the resulting amine (I-3) underwent Michael addition into the vinyl substrate providing the adduct (I-4). The reaction proceeds most readily under high solvent concentration using protic or aprotic solvents, either EtOH or DMF. Hydrolysis of the trifluoroacetate generated free amine (I-5). The amine was then coupled with 3-oxo-3,4-dihydro-2H-pyrido[3,2-b][1,4]thiazine-6-carbaldehyde through reductive amination forming (I-6), with 3-oxo-3,4-dihydro-2H-pyrido[3,2-b][1,4]thiazine-6-carboxylic acid forming amide (I-7) or with 3-oxo-3,4-dihydro-2H-pyrido[3,2-b][1,4]thiazine-6-sulfonyl chloride generating sulfonamide (I-8). 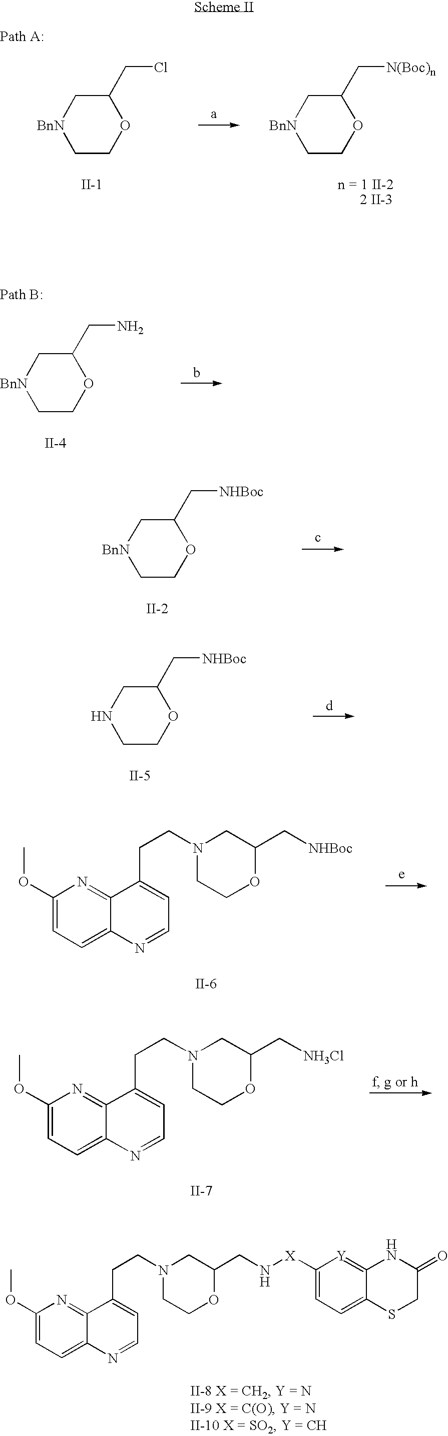
Reagents and conditions: (a) NaH, NHBoc₂, DMF, 140° C. (b) Boc₂O, CH₂Cl₂, 25° C. (c) H₂ (50 psi), 10% Pd—C, EtOH (d) 8-ethenyl-2-(methyloxy)-1,5-naphthyridine, DMF, 90° C. (e) 4M HCl in dioxane, MeOH, 25° C. (f) 3-oxo-3,4-dihydro-2H-pyrido[3,2-b][1,4]thiazine-6-carbaldehyde, DIPEA, Na₂SO₄, DCM-EtOH; then NaBH₄, 25° C. (g) 3-oxo-3,4-dihydro-2H-pyrido[3,2-b][1,4]thiazine-6-carboxylic acid, DIPEA, 1-(3-Dimethylaminopropyl)-3-ethylcarbodimide, DCM-DMF, 25° C. (h) DIPEA, 3-oxo-3,4-dihydro-2H-pyrido[3,2-b][1,4]thiazine-6-sulfonyl chloride, DCM, 25° C. morpholine (II-2) was prepared using two independent methods. In path A, the chloromethyl morpholine (II-1) (prepared according to Kato, S.; Morie, T.; Hino, K.; Kon, T.; Naruto, S.; Yoshida, N.; Karasawa, T.; Matsumoto, J. J. Med. Chem. 1990, 33, 1406) underwent nucleophilic displacement with an appropriate amine generating a mixture of mono and bis-Boc amine products (II-2 and II-3, 2:1). Alternatively, the aminomethyl morpholine (II-4) in path B (prepared according to the above reported procedure) was protected as the Boc carbamate and hydrogenation removed the benzyl protecting group providing the free amine (II-5). Subsequent Michael addition with 8-ethenyl-2-(methyloxy)-1,5-naphthyridine provided the adduct (II-6). The Boc group was removed and the resulting amine was coupled with 3-oxo-3,4-dihydro-2H-pyrido[3,2-b][1,4]thiazine-6-carbaldehyde through reductive amination forming (II-8), with 3-oxo-3,4-dihydro-2H-pyrido[3,2-b][1,4]thiazine-6-carboxylic acid forming amide (II-9) or with 3-oxo-3,4-dihydro-2H-pyrido[3,2-b][1,4]thiazine-6-sulfonyl chloride generating sulfonamide (II-10). 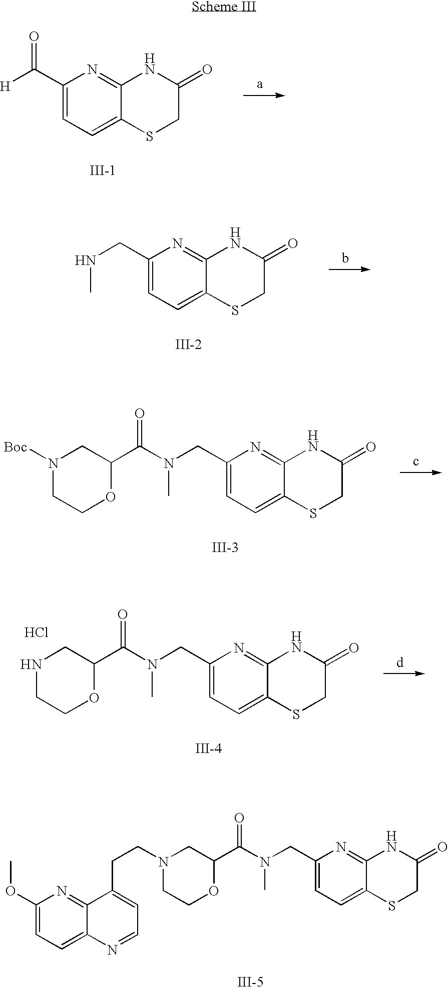
Reagents and conditions: (a) 2M MeNH₂ in MeOH, DCM-MeOH, 12 h, then NaBH₄, 25° C. (b) 4-{[(1,1-dimethylethyl)oxy]carbonyl}-2-morpholinecarboxylic acid, 1-(3-Dimethylaminopropyl)-3-ethylcarbodimide, 1-hydroxyenzotriazole, DCM-DMF, 25° C. (c) 4M HCl in dioxane, MeOH, 25° C. (d) 8-ethenyl-2-(methyloxy)-1,5-naphthyridine, DIPEA, DMF, 90° C.

The aldehyde (III-1) was transformed into the N-methyl amine (III-2) via reductive amination. The resulting amine was then coupled to the morpholine acid affording the amide (III-3). The Boc group was removed and subsequent Michael addition into 8-ethenyl-2-(methyloxy)-1,5-naphthyridine, as described in both Scheme I and II, provided the final compound (III-5). 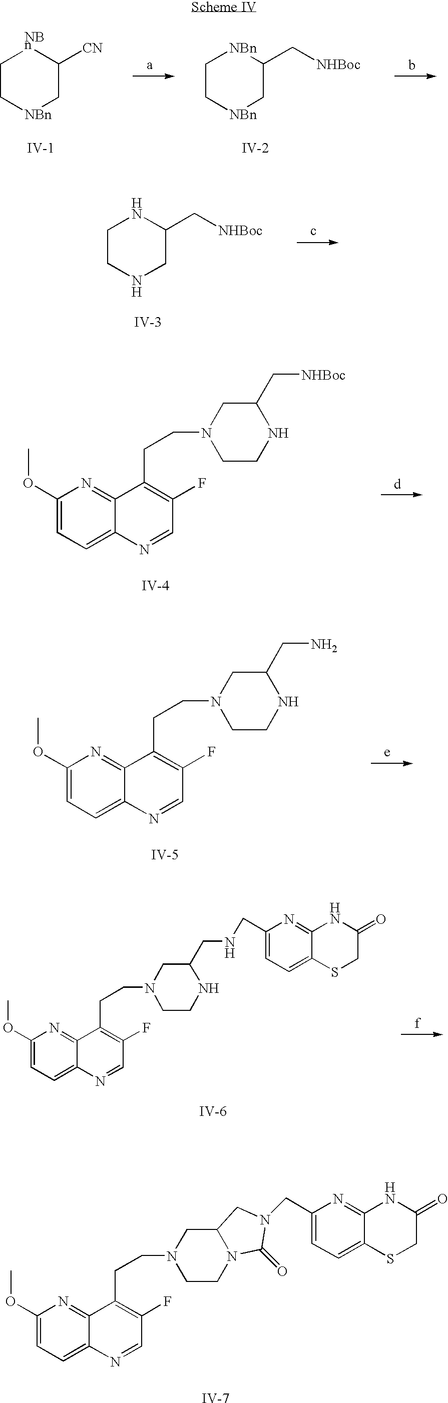
Reagents and conditions: (a) LAH, THF, 0-25° C.; then Boc₂O, THF, 25° C. (b) H₂ (50 psi), 10% Pd—C, EtOH (c) 8-ethenyl-7-fluoro-2-(methyloxy)-1,5-naphthyridine, EtOH, 85° C. (d) 4M HCl in dioxane, MeOH, 25° C. (e) 3-oxo-3,4-dihydro-2H-pyrido[3,2-b][1,4]thiazine-6-carbaldehyde, DIPEA, Na₂SO₄, DCM-EtOH; then NaBH₄, 25° C. (f) 20% phosgene in toluene, triethylamine, DCM, 0° C.

The cyano group of morpholine (IV-1) (prepared according to Godfroid, J.-J.; et al J. Med. Chem. 1999, 42, 9, 1587) was reduced to the amine and subsequently protected as the Boc carbamate (IV-2). Hydrogenation removed the benzyl groups providing the piperazine (IV-3). Subsequent Michael addition into 8-ethenyl-7-fluoro-2-(methyloxy)-1,5-naphthyridine yielded the adduct (IV-4). The Boc group was removed and the resulting amine (IV-5) was coupled through reductive amination with 3-oxo-3,4-dihydro-2H-pyrido[3,2-b][1,4]thiazine-6-carbaldehyde generating (IV-6). The resulting amine was then cyclized with phosgene to urea (IV-7). 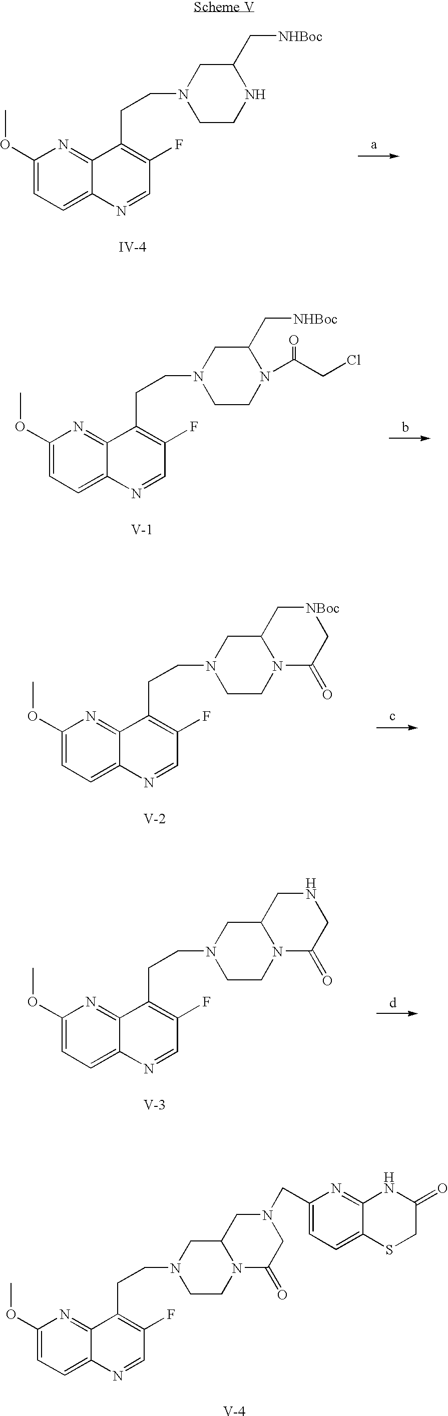
Reagents and conditions: (a) chloroacetyl chloride, Et₃N, THF, 0° C. (b) NaH, DMF-THF, 0-25° C. (c) 4M HCl in dioxane, MeOH, 25° C. (d) 3-oxo-3,4-dihydro-2H-pyrido[3,2-b][1,4]thiazine-6-carbaldehyde, Na₂SO₄, Na(OAc)₃BH, DCE, 25° C.

The piperazine (IV-4) [described in Scheme IV] was acetylated generating the amide (V-1). Treatment with NaH induced cyclization providing adduct (V-2). The Boc protecting group was then removed and the resulting amine underwent reductive amination with 3-oxo-3,4-dihydro-2H-pyrido[3,2-b][1,4]thiazine-6-carbaldehyde affording the final analog (V-4). 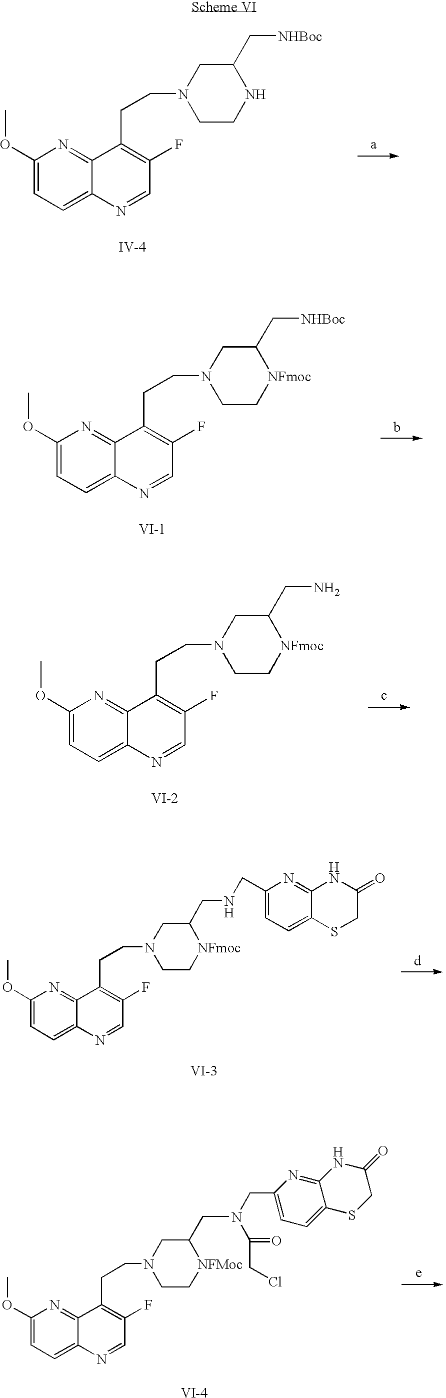 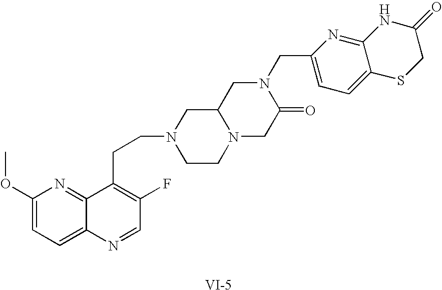
Reagents and conditions: (a) Fmoc-CI, Et₃N, DCM, 0° C. (b) 4M HCl in dioxane, MeOH, 25° C. (c) 3-oxo-3,4-dihydro-2H-pyrido[3,2-b][1,4]thiazine-6-carbaldehyde, Na₂SO₄, Na(OAc)₃BH, DCM-EtOH, 25° C. (d) Chloroacetyl chloride, Et₃N, THF, 0° C. (e) TBAF, THF, 0-25° C.

Piperazine (IV-4) [described in Scheme IV] was protected as the carbamate (VI-1). The Boc protecting group was removed and the resulting free amine underwent reductive amination using 3-oxo-3,4-dihydro-2H-pyrido[3,2-b][1,4]thiazine-6-carbaldehyde and the resulting secondary amine was acylated generating amide (VI-4). Exposure to a fluoride source served to remove the carbamate and cyclize the amine onto the chloroamide generating the final analog (VI-5). 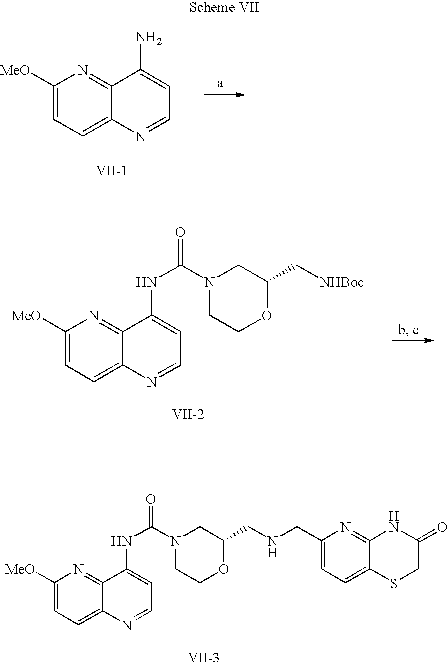
Reagents and conditions: (a) CDI, DMAP, CHCl₃, RT; then 1,1-dimethylethyl[(2R)-2-morpholinylmethyl]carbamate, DMF, 10° C. (b) 4M HCl in dioxane, MeOH, 25° C. (c) 3-oxo-3,4-dihydro-2H-pyrido[3,2-b][1,4]thiazine-6-carbaldehyde, DIPEA, Na₂SO₄, DCM-EtOH; then NaBH₄, 25° C.

Amine (VII-1) was coupled to an appropriate morpholine generating urea (VII-2). The Boc protecting group was removed and the resulting free amine was then coupled with 3-oxo-3,4-dihydro-2H-pyrido[3,2-b][1,4]thiazine-6-carbaldehyde through reductive amination generating the final analog (VII-3).

### General Experimental

Proton nuclear magnetic resonance (¹H NMR) spectra were recorded at 400 MHz, and chemical shifts are reported in parts per million (δ) downfield from the internal solvent standard CHCl₃ or MeOH. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. CDCl₃ is deuterochloroform and CD₃OD is tetradeuteromethanol. Mass spectra were obtained using electrospray (ES) ionization techniques. All temperatures are reported in degrees Celsius. E. Merck Silica Gel 60 F-254 thin layer plates were used for thin layer chromatography. Flash chromatography was carried out on E. Merck Kieselgel 60 (230-400 mesh) silica gel. Analytical HPLC was performed on Beckman chromatography systems. Preparative HPLC was performed using Gilson chromatography systems. ODS refers to an octadecylsilyl derivatized silica gel chromatographic support. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

### Preparation 1

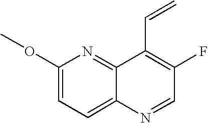

### Preparation of 8-ethenyl-7-fluoro-2-(methyloxy)-1,5-naphthyridine

### (a) (2-[(6-Methoxypyridin-3-ylamino)methylene]malonic acid diethyl ester

A solution of 5-amino-2-methoxypyridine (Aldrich, 100 g, 0.806 mole) and diethyl ethoxymethylenemalonate (Aldrich, 163 mL, 0.806 mole) in EtOH (1 L) was heated at reflux for 4 h, then was cooled to RT. Concentration to dryness gave the title compound (238 g, quantitative).

### (b) 6-Methoxy-4-oxo-1,4-dihydro-[1,5]naphthyridine-3-carboxylic acid ethyl ester

Dowtherm A (Fluka, 500 mL) was brought to boiling (250° C.) in a 2 L 3-neck flask fitted with a still-head and a reflux condenser. 2-[(6-Methoxypyridin-3-ylamino)methylene]malonic acid diethyl ester (100 g, 0.34 mole) was added portion-wise over 5 min. The solution was heated at reflux for an additional 15 min, allowing some solvent to distil over. The resulting solution was cooled to room temperature and diluted with hexane (750 mL). The mixture was cooled in ice for 1 h, then the brown solid was filtered off, washed with hexane, and dried under vacuum to afford the title compound (61.72 g, 73%).

### (c) 4-Bromo-6-methoxy-[1,5]naphthyridine-3-carboxylic acid ethyl ester

A suspension of 6-methoxy-4-oxo-1,4-dihydro-[1,5]naphthyridine-3-carboxylic acid ethyl ester (74.57 g, 300 mmol) in dry DMF (260 mL) under argon was stirred efficiently* in a water bath (to maintain approximately room temperature—may need slight ice-cooling on a large scale). Phosphorus tribromide (30.0 mL, 316 mmol) was added dropwise over 15 min and stirring was continued for an additional 30 min. Water (1 L) was added, followed by saturated sodium carbonate solution to pH 7. The solid was collected by suction filtration, washed with water and dried under vacuum over phosphorus pentoxide to give the title compound (83.56 g, 90%).

### (d) 4-Bromo-6-methoxy-[1,5]naphthyridine-3-carboxylic acid

2 N NaOH (300 mL, 600 mmol) was added dropwise over 30 min to a stirred solution of 4-bromo-6-methoxy-[1,5]naphthyridine-3-carboxylic acid ethyl ester (83.56 g, 268 mmol) in THF (835 mL). Stirring was continued overnight, at which time LC/MS showed that the saponification was complete. 2 N HCl was added to pH 6 and the THF was removed in vacuo. 2 N HCl was added to pH 2, then water (250 mL) was added, and the mixture was cooled thoroughly in ice. The solid was collected by suction filtration, washed with water and dried (first using a rotary evaporator at 50° C. and then under high vacuum at 50° C. overnight) to give the title compound (76.7 g, slightly over quantitative). This material was used without further purification.

### (e) 4-Bromo-6-methoxy-[1,5]naphthyridin-3-ylamine

A suspension of 4-bromo-6-methoxy-[1,5]naphthyridine-3-carboxylic acid (50 g, 177 mmol) in dry DMF (600 mL) was treated with triethylamine (222.5 mL, 1.60 mole), tert-butanol (265 mL, 2.77 mole) and diphenylphosphoryl azide (41.75 mL, 194 mmol). The reaction was stirred under argon at 100° C. for 1 h, then was cooled to room temperature and concentrated to low volume. Ethyl acetate and excess aqueous sodium bicarbonate solution were added, the mixture was shaken, and some insoluble solid was filtered off. The layers were separated and the organic phase washed with water (2×) and dried (MgSO₄). Concentration to dryness gave a crude mixture of 4-bromo-6-methoxy-[1,5]naphthyridin-3-ylamine (minor product) and (4-bromo-6-methoxy-[1,5]naphthyridin-3-ylamine)carbamic acid tert-butyl ester (major product) along with impurities.

Without further purification, this mixture was dissolved in CH₂Cl₂ (150 mL) and treated with trifluoroacetic acid (100 mL). The reaction was stirred for 3 h then was concentrated to dryness. The residue was partitioned between CHCl₃ and saturated sodium bicarbonate solution and the layers were separated. The aqueous phase was extracted with CHCl₃, and the combined organic fractions were dried (MgSO₄) and concentrated to low volume. The solid was collected by suction filtration, washed with a small volume of CHCl₃ and dried under vacuum to afford a first crop of the title compound (31.14 g). The filtrate was purified by flash chromatography on silica gel (30% EtOAc in CHCl₃) to afford further material (2.93 g, total=34.07 g, 76%). Alternatively, the filtrate was left at room temperature overnight and then filtered to give a second crop of the title compound (2.5 g).

### (f) 4-Bromo-6-methoxy-[1,5]naphthyridine-3-diazonium tetrafluoroborate

A solution of 4-bromo-6-methoxy-[1,5]naphthyridin-3-ylamine (25.2 g, 99.2 mmol) in dry THF (400 mL) was maintained at −5° C. while nitrosonium tetrafluoroborate (12.9 g, 110 mmol) was added portion-wise over 30 min (approximately 2 g portions). The reaction was continued for an additional 1 h at −5° C., at which time TLC* and LC/MS indicated that the reaction was complete. The orange solid was collected by suction filtration, washed with ice-cold THF and dried under vacuum to provide the title compound (31.42 g, 90%).

### (g) 4-Bromo-3-fluoro-6-methoxy-[1,5]naphthyridine

A suspension of 4-bromo-6-methoxy-[1,5]naphthyridine-3-diazonium tetrafluoroborate (31.42 g, 89.0 mmol) in decalin (mixed isomers, 500 mL) in a 2 L flask* was heated to 180° C. and held at this temperature for 5 min. The mixture was cooled and diluted with CHCl₃ (500 mL, to keep the product in solution), and the resulting mixture was stirred vigorously for 30 min to break up a black solid by-product. The mixture was then poured onto a column of silica gel and the column was eluted with CHCl₃ to remove decalin and then with 3% EtOAc/CHCl₃ to afford the title compound (9.16 g, 40%).

### (h) 8-ethenyl-7-fluoro-2-(methyloxy)-1,5-naphthyridine

To a solution of 8-bromo-7-fluoro-2-(methyloxy)-1,5-naphthyridine (2.0 g, 7.81 mmol), potassium carbonate (1.08 g, 7.81 mmol), tetrakis-triphenylphosphine (90 mg, 0.08 mmol) in DME (60 mL) and H₂O (20 mL) was added 2,4,6-trivinylcycloborane-pyridine complex (0.94 g, 3.91 mmol). After stirring for 10 h at 85° C. the reaction contents were concentrated and the product purified by chromatography (silica, 25% EtOAc in hexane) to give a low melting solid (1.43 g, 90%).

### Preparation 2

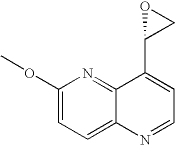

### Preparation of (S)-2-(6-Methoxy-[1,5]-naphthyridin-4-yl)oxirane

### (a) 4-Hydroxy-6-methoxy-[1,5]-naphthyridine

5-Amino-2-methoxypyridine (55 g, 0.44 mol) in methanol (1000 ml) with methyl propiolate (40 ml, 0.44 mol) was stirred for 48 h, then evaporated and the product purified by chromatography on silica gel (DCM) followed by recrystallisation from DCM-hexane (44.6 g, 48%).

The unsaturated ester (10.5 g, 0.05 mol) in warm Dowtherm A (50 ml) was added over 3 minutes to refluxing Dowtherm A, and after a further 20 minutes at reflux the mixture was cooled and poured into ether. The precipitate was filtered to give the title compound (6.26 g, 70%)

### (b) Bromomethyl-(6-methoxy-[1,5]-naphthyridin-4-yl)-ketone

4-Hydroxy-6-methoxy-[1,5]-naphthyridine (10 g, 0.057 mol) in DCM (200 ml) containing 2,6-lutidine (9.94 ml, 0.086 mol) and 4-dimethylaminopyridine (0.07 g, 0.0057 mol) was cooled in ice and treated with trifluoromethanesulfonic anhydride (10.5 ml, 0.063 mol). After stirring for 2.5 h the mixture washed with saturated ammonium chloride solution, dried, evaporated and purified on silica (DCM). The triflate (13.2 g, 0.044 mol) in DMF (200 ml) with TEA (12 ml, 0.086 mol), butyl vinyl ether (22 ml, 0.17 mol), 1,3-bis(diphenylphosphino)propane (1.77 g, 0.0044 mol) and palladium (II) acetate (0.97 g, 0.0044 mol) was heated at 60° C. for 3 h then evaporated and chromatographed on silica gel (DCM) to give a yellow solid (10.7 g, 95%). This was dissolved in THF (250 ml), water (40 ml) and treated with N-bromosuccinimide (7.4 g. 0.042 mol) for 1 h, then evaporated and chromatographed on silica gel (DCM) to give the ketone (10.42 g, 98%).

### (c) (R)-2-Bromo-1-(6-methoxy-[1,5]-naphthyridin-4-yl)ethanol

Bromomethyl-(6-methoxy-[1,5]-naphthyridin-4-yl)-ketone (6.6 g, 0.023 mol) in toluene was treated with (+)-B-chlorodiisopinocamphenylborane ((+)-DIP-chloride) (12 g, 0.037 mol) and stirred overnight, then diethanolamine (15 g, 0.14 mol) was added and the mixture was stirred for 3 h, filtered and evaporated. Chromatography on silica gel (ethyl acetate-hexane) gave the title compound as a white solid (4.73 g, 73%).

### (d) (R)-2-(6-Methoxy-[1,5]-naphthyridin-4-yl)oxirane

(R)-2-Bromo-1-(6-methoxy-[1,5]-naphthyridin-4-yl)ethanol (4.8 g, 0.017 mol) in MeOH (20 ml) was stirred with potassium carbonate (2.6 g, 0.019 mol) for 1 h, then evaporated and chromatographed on silica gel (ethyl acetate-hexane-dichloromethane) to give a solid (3.14 g, 92%), (91% ee by chiral HPLC). LC/MS (+ve ion electrospray) m/z 203 (M+H+).

### Preparation 3

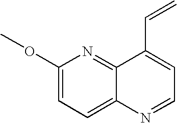

### Preparation of 8-ethenyl-2-(methyloxy)-1,5-naphthyridine

To a solution of 6-(methyloxy)-1,5-naphthyridin-4-yl trifluoromethanesulfonate (from Prep. 2b) (5.0 g, 16.23 mmol) in DME (80 mL) and H₂O (40 mL) was added trivinyl boronate (1.96 g, 8.1 mmol), K₂CO₃ (2.23 g, 16.23 mmol) and Pd(PPh₃)₄ (0.19 g, 0.16 mmol). After 3 h at 90° C. under N₂, the reaction solution was concentrated under vacuum and purified on silica (hexane/EtOAc, 4:1) to give the title compound as a yellow oil (2.44 g, 81%): LC/MS (m/z) (ES) 187 (M+H)⁺.

### Preparation 4

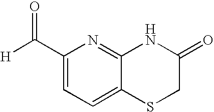

### Preparation of 3-Oxo-3,4-dihydro-2H-pyrido[3,2-b][1,4]thiazine-6-carboxaldehyde

### (a) Methyl 3-oxo-3,4-dihydro-2H-pyrido[3,2-b][1,4]thiazine-6-carboxylate

A solution of ethyl 2-mercaptoacetate (1.473 mL) in DMF (48 mL) was ice-cooled and treated with sodium hydride (540 mg of a 60% dispersion in oil). After 1 h methyl 6-amino-5-bromopyridine-2-carboxylate (3 g) (T. R. Kelly and F. Lang, J. Org. Chem. 61, 1996, 4623-4633) was added and the mixture stirred for 16 h at room temperature. The solution was diluted with EtOAc (1 L), washed with water (3×300 mL), dried and evaporated to about 10 mL. The white solid was filtered off and washed with a little EtOAc to give the ester (0.95 g); LC/MS (APCI⁻) m/z 223 ([M−H]⁻, 100%).

### (b) 3-Oxo-3,4-dihydro-2H-pyrido[3,2-b][1,4]thiazine-6-carboxylic acid

A solution of Methyl 3-oxo-3,4-dihydro-2H-pyrido[3,2-b][1,4]thiazine-6-carboxylate (788 mg) in dioxan (120 ml)/water (30 mL) was treated dropwise over 2 h with 0.5M NaOH solution (8 mL) and stirred overnight. After evaporation to approx. 3 ml, water (5 mL) was added and 2M HCl to pH4. The precipitated solid was filtered off, washed with a small volume of water and dried under vacuum to give a solid (636 mg); LC/MS (APCI⁻) m/z 209 ([M−H]⁻, 5%), 165 ([M-COOH]⁻, 100%).

### (c) 6-Hydroxymethyl-3-oxo-3,4-dihydro-2H-pyrido[3,2-b][1,4]thiazine

A solution of 3-oxo-3,4-dihydro-2H-pyrido[3,2-b][1,4]thiazine-6-carboxylic acid (500 mg) in THF (24 mL) with triethylamine (0.396 mL) was cooled to −10° C. and isobutyl chloroformate (0.339 ml) was added. After 20 minutes the suspension was filtered through kieselguhr into an ice-cooled solution of sodium borohydride (272 mg) in water (8 mL), the mixture stirred 30 minutes and the pH reduced to 7 with dilute HCl. The solvent was evaporated and the residue triturated under water. The product was filtered and dried under vacuum to give a white solid (346 mg); LC/MS (APCI⁻) m/z 195 ([M−H]⁻, 50%), 165 (100%).

### (d) 3-Oxo-3,4-dihydro-2H-pyrido[3,2-b][1,4]thiazine-6-carboxaldehyde

A solution of 6-hydroxymethyl-3-oxo-3,4-dihydro-2H-pyrido[3,2-b][1,4]thiazine (330 mg) in dichloromethane (30 mL)/THF (30 mL) was treated with manganese dioxide (730 mg) and stirred at room temperature. Further manganese dioxide was added after 1 h (730 mg) and 16 h (300 mg). After a total of 20 h the mixture was filtered through kieselguhr and the filtrate evaporated. The product was triturated with EtOAc/hexane (1:1) and collected to give a solid (180 mg); LC/MS (APCI⁻) m/z 195 ([M−H]⁻, 95%), 165 (100%).

### Preparation 5

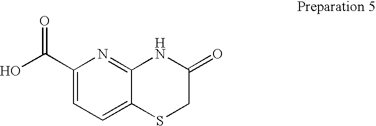

### Preparation of 3-oxo-3,4-dihydro-2H-pyrido[3,2-b][1,4]thiazine-6-carboxylic acid

This acid was prepared from 3-oxo-3,4-dihydro-2H-pyrido[3,2-b][1,4]thiazine-6-carboxaldehyde (from Prep. 4d) (890 mg) by oxidation with Oxone (potassium peroxymonosulphate) (3.1 g) in a DMF solution (50 mL). After 1.5 h at room temperature, dilution with water (50 mL), filtration and drying in vacuo afforded the acid as a white solid (750 mg, 77%).

### Preparation 6

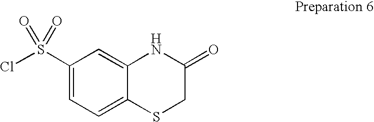

### Preparation of 3-oxo-3,4-dihydro-2H-1,4-benzothiazine-6-sulfonyl chloride

To an ice-cold solution of chlorosulfonic acid (22 mL, 33.1 mmol) was added the benzothiazinone (6 g, 36.3 mmol) portion-wise. The dark blue solution was warmed to 25° C. over 1 h, then heated at 45° C. for 2 h. After cooling, addition of the solution to ice-water resulted in the formation of a white precipitate. The solid was filtered, washed with H₂O/hexane and dried affording the title compound as a white solid (8.46 g, 88%); MS (APCI+) m/z 246 (M+H)⁺.

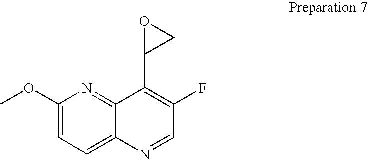

### Preparation of 7-fluoro-2-(methyloxy)-8-[(2S)-2-oxiranyl]-1,5-naphthyridine

### a) 1-[3-fluoro-6-(methoxy)-1,5-naphthyridin-4-yl]-1,2-ethanediol

To a solution of AD-mix-β (50 g) in tert-butanol/water (200 mL/200 mL), cooled in an ice-bath for 30 minutes, 8-ethenyl-7-fluoro-2-(methyloxy)-1,5-naphthyridine (prepared as Preparation 1) (8 g, 39.2 mmol) was added and the reaction mixture was stirred at room temperature for 48 hours. Sodium sulfite (75 g) was added and the mixture was stirred for a further 30 minutes. It was extracted with diethyl ether then several times with 10% methanol in chloroform. The organic extract was evaporated under vacuum to afford the desired product as an oil (8.93 g, 96%). MS (+ve ion electrospray) m/z 239 (MH+). enantiomeric excess=44%, as determined by chiral analytical HPLC

### b) 2-[3-fluoro-6-(methoxy)-1,5-naphthyridin-4-yl]-2-hydroxyethyl 4-methylbenzenesulfonate

To a solution of diol (a) (16.5 g, 6.93 mmol) in DCM (200 mL), triethylamine (10 mL) and dibutyltin oxide (350 mg) was added tosyl chloride (13.2 g, 6.94 mmol). After 3 hours, the mixture was diluted with water/sodium bicarbonate and extracted several times with chloroform. The combined organic extracts were dried over magnesium sulfate and evaporated under vacuum. The residue was chromatographed on silica gel eluting with 20-30% ethyl acetate in chloroform to afford the desired product (20.3 g, 75%). MS (+ve ion electrospray) m/z 393 (MH+).

### c) 7-fluoro-2-(methoxy)-8-(2-oxiranyl)-1,5-naphthyridine

To a suspension of tosylate (b) (10.5 g, 26.7 mmol) in anhydrous methanol (160 mL), cooled in an ice-bath, potassium carbonate (7.03 g, 50.9 mmol) was added. After 15 minutes with cooling, the mixture was stirred at room temperature for a further 1.75 hours. It was then diluted with water, extracted several times with dichloromethane, dried over magnesium sulfate and evaporated under vacuum. The residue was chromatographed on silica gel eluting with dichlorometnane, chloroform then 20% ethyl acetate in chloroform to afford the title product as an oil (5.55 g, 94%). MS (+ve ion electrospray) m/z 221 (MH+).

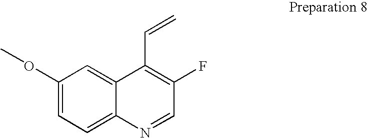

### Preparation of 4-ethenyl-3-fluoro-6-(methyloxy)quinoline

### a) 4-Hydroxy-6-methoxy-quinoline-3-carboxylic acid ethyl ester

A solution of 4-methoxyaniline (40 g, 0.32 mole) and diethyl ethoxymethylenemalonate (65 mL, 0.32 mole) in Dowtherm A (500 mL) was heated at reflux in a flask fitted with side-arm and condenser, and heating was continued until all the ethanol had distilled off (ca. 0.5 hr). The solution was cooled and pentane was added to give a sticky precipitate. The solvents were decanted off and the residue was treated with more pentane and allowed to stand overnight. The solid was filtered off and washed well with pentane to give the title compound (62.4 g; 78%, contains traces of Dowtherm A).

### b) 4-Bromo-6-methoxy-quinoline-3-carboxylic acid ethyl ester

PBr₃ (64.5 g, 22.5 mL, 0.239 mole) was added dropwise to a stirred, ice cold suspension of 4-hydroxy-6-methoxy-quinoline-3-carboxylic acid ethyl ester (59 g, 0.239 mole) in DMF (750 mL); the temperature rose to 15-20° C. for 30 min and then dropped to ca. 5° C. (the starting material dissolved fairly quickly and a new solid precipitated out). After 3 hr the solid was collected, washed sequentially with cold DMF, hexane, and water, then was dried at 40° C. in vacuo overnight to give the title compound (41 g, 78%): LC/MS (ES) m/e 310/312 (M+H)⁺.

### c) 4-Bromo-6-methoxyquinoline-3-carboxylic acid

4-Bromo-6-methoxy-quinoline-3-carboxylic acid ethyl ester (41 g, 0.132 mole), partially dissolved in THF (600 mL), was treated dropwise with aqueous 2 M sodium hydroxide (198.4 mL, 0.396 mole). After 24 hr, the reaction was complete by TLC (2% MeOH/CH₂Cl₂). The mixture was neutralized with 5 M HCl then the THF was removed in vacuo. The residue was dissolved in water and acidified with 5 M HCl. The solid product was collected under suction, washed well with water, and dried in vacuo to give the title compound (34 g, 91%) as a white solid: MS (ES) m/e 282/284 (M+H)⁺.

### d) (4-Bromo-6-methoxy-quinolin-3-yl)-carbamic acid tert-butyl ester

To a solution of 4-Bromo-6-methoxyquinoline-3-carboxylic acid (34 g, 0.121 mole), triethylamine (141 mL) and tert-butanol (181 mL) in dry DMF (400 mL) was added diphenylphosphoryl azide (36.6 g, 28.6 mL, 0.133 mole). The mixture was heated at 100° C. for 1 h (see Note), then cooled and concentrated. The residue was dissolved in CH₂Cl₂ and washed with water (some insoluble material was removed by filtration). The aqueous phase was extracted with dichloromethane and the combined organics were dried (Na₂SO₄) and concentrated. Chromatography on silica gel (1 kg, 1:1 ether/light petroleum ether) gave the carbamate (22.7 g, 53%): MS (ES) m/e 309/311 (M+H)⁺, 354/6.

Further elution with ether gave several mixed fractions then pure 3-amino-4-bromo-6-methoxyquinoline (2.0 g, 6.5%): MS (ES) m/e 309/311 (M+H)⁺, 254/6.

### e) 3-Amino-4-bromo-6-methoxyquinoline

(4-Bromo-6-methoxy-quinolin-3-yl)-carbamic acid tert-butyl ester (22.7 g, 0.0643 mole) was dissolved in CH₂Cl₂ (200 mL) and treated with trifluoroacetic acid (100 mL). After 3.5 hr at RT, the mixture was concentrated and the residue was dissolved in water. The solution was made basic with aqueous sodium carbonate. The precipitate was filtered off, washed with water, and dried at 40° C. in vacuo overnight, to give the title compound (16.46 g, 101%) as a white solid: MS (ES) m/e 254/256 (M+H)⁺.

### f) 4-Bromo-3-methoxyquinolin-3-yl-diazonium tetrafluoroborate

3-Amino-4-bromo-6-methoxyquinoline (18.4 g, 0.0727 mole) was dissolved in dry THF (250 mL) and the solution was cooled to −8° C. (EtOH-ice bath). Nitrosonium tetrafluoroborate (9.34 g, 0.08 mole) was added in portions over 10 min, keeping the temperature less than −2° C. The mixture was stirred at −5 to 0° C. for 30 min, then the yellow precipitate was filtered off and washed sequentially with cold THF and hexane. Drying in vacuo gave the title compound (19.4 g, 76%) an insoluble orange-yellow solid.

### g) 4-Bromo-3-fluoro-6-methoxyquinoline

A well stirred solution of decahydronaphthalene (mixed isomers, 120 mL) was heated to ca. 167-170° C. (internal temperature) and the diazonium tetrafluoroborate salt (6.0 g) was added portion-wise over 30 sec, when the solid turned black. The reaction mixture was immediately cooled and the decahydronaphthalene was filtered off. The filtrate was saved for further processing. The residue was extracted with dichloromethane (3×). Some insoluble material remained. The solution was concentrated and the residue was chromatographed on silica gel (CH₂Cl₂ then CHCl₃) to give the title compound (1.1 g) as a white solid: MS (ES) m/e 256/258 (M+H)⁺, Rt=2.65 min. About 4% of a dibromo impurity was present: MS (ES) m/e 316/318/320 (M+H)⁺Rt=2.94 min.

The decahydronaphthalene solution was treated with excess ethereal HCl and the solid hydrochloride salt was collected and washed with hexane. This was converted to the free base by reaction with aqueous sodium carbonate followed by extraction with CH₂Cl₂. This gave additional title compound (0.87 g; total yield=1.97 g, 45%).

### h) 4-ethenyl-3-fluoro-6-(methyloxy)quinoline

4-Bromo-3,fluoro-6-(methoxy)quinoline (2.3 mmol) in DME (26 mL) under argon, was treated with tetrakis(triphenylphosphine)palladium(0) (0.13 g, 0.115 mmol) and the mixture stirred at room temperature for 20 minutes. Anhydrous potassium carbonate (0.32 g, 2.3 mmol), water (7 mL), and vinylborane:pyridine complex (see F. Kerins and D O'Shea J. Org. Chem. 2002, 67, 4968-4971) (0.22 g, 0.92 mmol) were added and the mixture was heated at 100° C. for 2 hr. It was cooled, diluted with water and extracted with ether, dried over magnesium sulfate and evaporated to dryness. After work-up the product was chromatographed on silica gel, eluting with 10% methanol in DCM to afford a white solid (0.44 g, 90%). MS (+ve ion electrospray) m/z 203 (MH+).

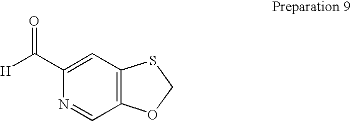

### Preparation of [1,3]Oxathiolo[5,4-c]pyridine-6-carbaldehyde

### a) 2-(hydroxymethyl)-5-({[4-(methoxy)phenyl]methyl}oxy)-4(1H)-pyrone

To a solution of Kojic acid (50 g, 0.352 mol) in DMF (650 mL) under an argon atmosphere, cooled to 0° C., was added a solution of potassium t-butoxide (39.5 g, 0.352 mol) in DMF (100 mL) and the resultant suspension was vigorously stirred (overhead stirring) for 1 hour at 5-10° C. 4-methoxybenzyl chloride was added dropwise and the mixture was heated to 50° C. for 30 hours, followed by 90° C. for 5 hours, after which the mixture was evaporated to a minimum volume of DMF. 750 mL of distilled water was added and the mixture refrigerated overnight. The resultant solid was collected by filtration and dried in vacuo at 50° C. to afford the product as a light brown solid (85 g, 64%); MS (+ve ion electrospray) m/z 263 (M+H+).

### b) 2-(hydroxymethyl)-5-({[4-(methoxy)phenyl]methyl}oxy)-4(1H)-pyridinone

To a suspension of 2-(hydroxymethyl)-5-({[4-(methoxy)phenyl]methyl}oxy)-4(1H)-pyrone (40 g, 153 mmol) in ethanol (105 mL) was added concentrated aqueous ammonia (295 mL) and refluxed for 18 hours. The mixture was cooled, then refrigerated for 3 hours, and cooled in an ice-bath for 45 minutes. The solid was filtered off, washed with cold ethanol, followed by cold petroleum ether and dried in vacuo to afford the product as brown solid (26.21 g, 66%).

### c) [5-({[4-(methoxy)phenyl]methyl}oxy)-4-oxo-1,4-dihydro-2-pyridinyl]methyl acetate

A solution of 2-(hydroxymethyl)-5-({[4-(methoxy)phenyl]methyl}oxy)-4(1H)-pyridinone (26 g, 0.1 mol) in pyridine (150 mL) was cooled to 5° C. and treated with acetyl chloride (10.48 ml, 0.149 mol). The reaction mixture was stirred and allowed to warm to room temperature then heated at 60° C. for 18 hours. Pyridine was evaporated under vacuum and the residue was triturated with water (250 mL), cooled in an ice-bath for 30 minutes. The solid formed was filtered off, washed with cold water and dried in vacuo to afford the product as a solid (15.7 g, 50%); MS (+ve ion electrospray) m/z 304 (MH+).

### d) (5-({[4-(methoxy)phenyl]methyl}oxy)-4-{[(trifluoromethyl)sulfonyl]oxy}-2-pyridinyl)methyl acetate

[5-({[4-(methoxy)phenyl]methyl}oxy)-4-oxo-1,4-dihydro-2-pyridinyl]methyl acetate (25 g, 82 mmol) was dissolved in dry dichloromethane (600 mL). Triethylamine (23 mL, 164 mmol) was added and the reaction cooled to 0° C. Trifluoromethane sulfonic anhydride (21 mL, 123 mmol) was added dropwise and the reaction left to stir at room temperature overnight. The reaction was poured into water, the organic layer collected and dried (MgSO₄). The crude product was chromatographed on silica eluting with 10-20% Ethyl acetate in hexane. Product containing fractions were combined and dried to afford the product as a solid (24.95 g, 70%); MS (+ve ion electrospray) m/z 436 (MH+).

### e) [4-[(1,1-dimethylethyl)thio]-5-({[4-(methoxy)phenyl]methyl}oxy)-2-pyridinyl]methyl acetate

To a solution of (5-({[4-(methoxy)phenyl]methyl}oxy)-4-{[(trifluoromethyl)sulfonyl]oxy}-2-pyridinyl)methyl acetate (10 g, 23 mmol) in anhydrous toluene, (R)-(+)-2,2 bis(diphenylphosphino)-1,1-binaphthyl (312 mg, 0.4 mmol) was added. The reaction mixture was degassed before adding palladium acetate (103 mg, 0.4 mmol). Sodium 2-methyl-2-propanethiolate was added, the system degassed again and the reaction mixture was stirred at 60° C. for 3 hours, under argon atmosphere then at 70 oC for a further 18 hours. The reaction mixture was filtered and the filtrate was evaporated under vacuum. The residue was partitioned between ethyl acetate and water. The aqueous layer was extracted several times with ethyl acetate. The combined organic extracts were dried over magnesium sulfate and evaporated under vacuum. The residue was chromatographed on silica gel eluting with 20-35% ethyl acetate in hexane to afford the product as an oil (9.1 g, 100%); MS (+ve ion electrospray) m/z 376 (MH+).

### f) {4-[(1,1-dimethylethyl)thio]-5-hydroxy-2-pyridinyl}methyl acetate

A solution of [4-[(1,1-dimethylethyl)thio]-5-({[4-(methoxy)phenyl]methyl}oxy)-2-pyridinyl]methyl acetate (9 g, 24 mmol) in dichloromethane (100 mL) was treated with triethylsilane (3.86 mL, 24 mmol). The reaction mixture was stirred for 10 minutes before adding trifluoroacetic acid (10 mL). The reaction mixture was stirred at room temperature for 3 hours under argon atmosphere. The solvents were evaporated under vacuum. The residue was taken up in dichloromethane and chromatographed on silica gel eluting with 10%-30% ethyl acetate in hexane to afford the product as an oil (5.1 g, 83%); MS (+ve ion electrospray) m/z 256 (MH+).

### g) 6-(hydroxymethyl)-4-mercapto-3-pyridinol

{4-[(1,1-dimethylethyl)thio]-5-hydroxy-2-pyridinyl}methyl acetate (2.5 g, 9.8 mmol) was dissolved in concentrated HCl and the mixture was heated at 80° C. for 18 hours. The solvent was evaporated under vacuum and the residue was triturated with diethyl ether to afford the product as a solid (1.35 g, 88%); MS (+ve ion electrospray) m/z 158 (MH+).

### h) [1,3]oxathiolo[5,4-c]pyridine-6-methanol

To a solution of 6-(hydroxymethyl)-4-mercapto-3-pyridinol (500 mg, 3.2 mmol) in anhydrous DMF, potassium carbonate was added. The reaction mixture was stirred for 10 minutes and dibromomethane (0.44 mL, 6.4 mmol) was added. The reaction mixture was stirred at 70° C. for 18 hours under an argon atmosphere. DMF was removed in vacuo and the residue was partitioned between 5% MeOH in dichloromethane and water. The aqueous layer was extracted several times with 5% methanol in dichloromethane. The combined organic extracts were dried over magnesium sulfate and evaporated under vacuum. The residue was chromatographed on silica gel eluting with 3-5% methanol in dichloromethane to afford the product as a solid (381 mg, 70%); MS (+ve ion electrospray) m/z 170 (MH+).

### i) [1,3]oxathiolo[5,4-c]pyridine-6-carbaldehyde

[1,3]oxathiolo[5,4-c]pyridine-6-methanol (0.92 g, 5.44 mmole) was treated with manganese (IV) oxide (3.83 g, 44 mmole) at RT in DCM (50 mL) to afford the aldehyde (567 mg, 62%) as a solid; MS (+ve ion electrospray) m/z 168 (MH+).

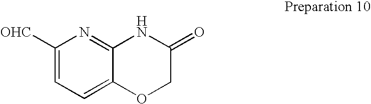

### Preparation of 3-Oxo-3,4-dihydro-2H-pyrido[3,2-b][1,4]oxazine-6-carboxaldehyde

### a) 2-Bromo-5-hydroxy-6-nitropyridine

3-Hydroxy-2-nitropyridine (20 g, 0.143 mole) was dissolved in methanol (400 mL) and a solution of 25% sodium methoxide in methanol (33 mL, 0.13 mole) was added at room temperature. The mixture was stirred for 30 min, then was cooled to 0° C., and bromine (7.2 mL, 0.14 mole) was added slowly. The reaction was stirred at 0° C. for 30 min, then was quenched with glacial AcOH (2.5 mL). The solvent was removed in vacuo to afford material (30 g, 96%), which was used without further purification.

MS (ES) m/z 219.0 (M+H)⁺.

### b) Ethyl(6-bromo-2-nitro-pyridin-3-yloxy)acetate

2-Bromo-5-hydroxy-6-nitropyridine (30 g, 0.14 mole) was suspended in acetone (200 ml), and potassium carbonate (39 g, 0.28 mole) was added, followed by ethyl bromoacetate (15.7 ml, 0.14 mmole). The reaction was heated at reflux for 10 hr, then was cooled to room temperature and diluted with Et₂O. The precipitate was removed by suction filtration, and the filtrate was concentrated in vacuo to afford material (38 g, 89%), which was used without further purification; MS (ES) m/z 305.0 (M+H)⁺.

### c) 6-Bromo-4H-pyrido[3,2-b][1,4]oxazin-3-one

Ethyl(6-bromo-2-nitro-pyridin-3-yloxy)acetate (38 g, 0.125 mole) was dissolved in glacial AcOH (150 mL), and iron powder (20 g, 0.36 mole) was added. The mixture was mechanically stirred and heated at 90° C. for 5 hr, then was cooled to room temperature and diluted with EtOAc (300 mL). The mixture was filtered through a pad of silica gel and the filtrate was concentrated in vacuo and the residue recrystallized from MeOH (15 g, 52%); MS (ES) m/z 229.0 (M+H)⁺.

### d) 6-((E)-Styryl)-4H-pyrido[3,2-b][1,4]oxazin-3-one

6-Bromo-4H-pyrido[3,2-b][1,4]oxazin-3-one (6.0 g, 26.3 mmole) and trans-2-phenylvinylboronic acid (3.9 g, 26.3 mmole) were dissolved in 1,4-dioxane (150 mL) and the solution was degassed with argon. (Ph₃P)₄Pd (230 mg, 0.2 mmole) was added, followed by a solution of potassium carbonate (6.9 g, 50 mmole) in H₂O (20 mL). The reaction was heated at reflux under argon overnight, then was cooled to room temperature and diluted with EtOAc (200 mL). The solution washed sequentially with H₂O and brine, dried (Na₂SO₄), and concentrated in vacuo. The solid residue was purified by flash chromatography on silica gel (5-10% EtOAc/CHCl₃) to afford a solid (2.5 g, 38%).

MS (ES) m/z 253.0 (M+H)⁺.

### e) 3-Oxo-3,4-dihydro-2H-pyrido[3,2-b][1,4]oxazine-6-carboxaldehyde

6-((E)-Styryl)-4H-pyrido[3,2-b][1,4]oxazin-3-one (1.2 g, 4.8 mmole) was dissolved in CH₂Cl₂ (200 mL) and the solution was cooled to −78° C. Ozone was bubbled through the solution with stirring until a pale blue color appeared, then the excess ozone was removed by bubbling oxygen through the solution for 15 min. Dimethylsulfide (1.76 mL, 24 mmole) was added to the solution, and the reaction was stirred at −78° C. for 3 hr, then at room temperature overnight. The solvent was removed in vacuo, and the residue was triturated with Et₂O (50 mL). The collected solid washed with additional Et₂O and dried to afford a solid (700 mg, 82%).

MS (ES) m/z 179.0 (M+H)⁺.

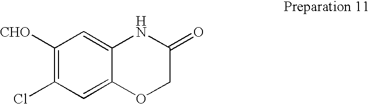

### Preparation of 7-chloro-3-oxo-3,4-dihydro-2H-1,4-benzoxazine-6-carbaldehyde

### a) 2-Bromo-5-hydroxy-6-nitropyridine

3-Hydroxy-2-nitropyridine (20 g, 0.143 mole) was dissolved in methanol (400 mL) and a solution of 25% sodium methoxide in methanol (33 mL, 0.13 mole) was added at room temperature. The mixture was stirred for 30 min, then was cooled to 0° C., and bromine (7.2 mL, 0.14 mole) was added slowly. The reaction was stirred at 0° C. for 30 min, then was quenched with glacial AcOH (2.5 mL). The solvent was removed in vacuo to afford material (30 g, 96%), which was used without further purification.

MS (ES) m/z 219.0 (M+H)⁺.

### b) Ethyl(6-bromo-2-nitro-pyridin-3-yloxy)acetate

The hydroxypyridine (30 g, 0.14 mole) was suspended in acetone (200 ml), and potassium carbonate (39 g, 0.28 mole) was added, followed by ethyl bromoacetate (15.7 ml, 0.14 mmole). The reaction was heated at reflux for 10 hr, then was cooled to room temperature and diluted with Et₂O. The precipitate was removed by suction filtration, and the filtrate was concentrated in vacuo to afford material (38 g, 89%), which was used without further purification.

MS (ES) m/z 305.0 (M+H)⁺.

### c) 6-Bromo-4H-pyrido[3,2-b][1,4]oxazin-3-one

The nitropyridine (38 g, 0.125 mole) was dissolved in glacial AcOH (150 mL), and iron powder (20 g, 0.36 mole) was added. The mixture was mechanically stirred and heated at 90° C. for 5 hr, then was cooled to room temperature and diluted with EtOAc 300 mL). The mixture was filtered through a pad of silica gel and the filtrate was concentrated in vacuo and the residue recrystallized from MeOH (15 g, 52%).

MS (ES) m/z 229.0 (M+H)⁺.

### d) 6-((E)-Styryl)-4H-pyrido[3,2-b][1,4]oxazin-3-one

The bromopyridine (10c) (6.0 g, 26.3 mmole) and trans-2-phenylvinylboronic acid (3.9 g, 26.3 mmole) were dissolved in 1,4-dioxane (150 mL) and the solution was degassed with argon. (Ph₃P)₄Pd (230 mg, 0.2 mmole) was added, followed by a solution of potassium carbonate (6.9 g, 50 mmole) in H₂O (20 mL). The reaction was heated at reflux under argon overnight, then was cooled to room temperature and diluted with EtOAc (200 mL). The solution washed sequentially with H₂O and brine, dried (Na₂SO₄), and concentrated in vacuo. The solid residue was purified by flash chromatography on silica gel (5-10% EtOAc/CHCl₃) to afford a solid (2.5 g, 38%).

MS (ES) m/z 253.0 (M+H)⁺.

### e) 3-Oxo-3,4-dihydro-2H-pyrido[3,2-b][1,4]oxazine-6-carboxaldehyde

The pyridine (10d) (1.2 g, 4.8 mmole) was dissolved in CH₂Cl₂ (200 mL) and the solution was cooled to −78° C. Ozone was bubbled through the solution with stirring until a pale blue color appeared, then the excess ozone was removed by bubbling oxygen through the solution for 15 min. Dimethylsulfide (1.76 mL, 24 mmole) was added to the solution, and the reaction was stirred at −78° C. for 3 hr, then at room temperature overnight. The solvent was removed in vacuo, and the residue was triturated with Et₂O (50 mL). The collected solid washed with additional Et₂O and dried to afford a solid (700 mg, 82%); MS (ES) m/z 179.0 (M+H)⁺.

### f) 6-Bromo-7-chloro-4H-pyrido[3,2-b][1,4]oxazin-3-one

6-Bromo-4H-pyrido[3,2-b][1,4]oxazin-3-one (20 g, 87.7 mmole) was dissolved in DMF (175 mL) and cooled in an ice bath. Chlorine gas was then slowly bubbled in for 45 minutes, and then the saturated solution was stirred in the ice bath for 2 hours. The mixture was purged with nitrogen and slowly added with stirring to 1 L of ice water which contained 100 g of Na₂SO₃, making sure to keep the temperature <15° C. After stirring 30 minutes the product was filtered, washed thoroughly with water and dried to afford (22.5 g, 98%) of a white solid.

¹H NMR (400 MHz, DMSO-d6): 4.76 (2H, s,), 7.78 (1H, s), 11.71 (1H, s).

### g) 7-Chloro-6-((E)-styryl)-4H-pyrido[3,2-b][1,4]oxazin-3-one

6-Bromo-7-chloro-4H-pyrido[3,2-b][1,4]oxazin-3-one (22 g, 83.7 mmole) and trans-2-phenylvinylboronic acid (17.33 g, 117 mmole) were dissolved in 1,4-dioxane (300 mL) and the solution was degassed with argon. (Ph₃P)₄Pd (1.9 g, 2 mole %) was added, followed by a solution of potassium hydrogen carbonate (21 g, 210 mmole) in H₂O (100 mL). The reaction was heated at reflux under argon overnight, then was cooled to room temperature and diluted with ethyl acetate (1 L). The solution washed sequentially with H₂O and brine, dried (Na₂SO₄), and concentrated in vacuo. The residue was slurried with chloroform (120 mL), then diluted with diethyl ether (100 mL). The precipitated product was collected by filtration and washed with ether to provide the product (16.4 g, 68%) as an off-white solid.

¹H NMR (400 MHz, DMSO-d6): 4.71 (2H, s), 7.32-7.46 (3H, m), 7.54-7.74 (4H, m), 11.6 (1H, s).

### h) 7-Chloro-3-oxo-3,4-dihydro-2H-pyrido[3,2-b][1,4]oxazine-6-carboxaldehyde

7-Chloro-6-((E)-styryl)-4H-pyrido[3,2-b][1,4]oxazin-3-one (8.0 g, 27.9 mmole) was dissolved in a mixture of DMF (400 mL) and methanol (40 mL), and the solution was cooled to −78° C. Ozone was bubbled through the solution with stirring for 45 minutes, then the excess ozone was removed by bubbling oxygen through the solution for 30 min. Dimethylsulfide (21 mL, 279 mmole) was added to the solution, and the reaction was stirred at −78° C. for 3 hr, then at room temperature overnight. The solvent was removed in vacuo, and the residue was triturated with Et₂O (150 mL). The collected solid was washed with additional Et₂O and dried to afford a white solid (4 g, 68%).

¹H NMR (400 MHz, DMSO-d6): 4.86 (2H, m), 7.73 (1H, s); 10.05 (1H, s), 11.84 (1H, s).

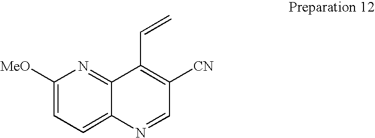

### Preparation of 4-ethenyl-6-(methyloxy)-1,5-naphthyridine-3-carbonitrile

### a) (2-[(6-Methoxypyridin-3-ylamino)methylene]malonic acid diethyl ester

A solution of 5-amino-2-methoxypyridine (Aldrich, 100 g, 0.806 mole) and diethyl ethoxymethylenemalonate (Aldrich, 163 mL, 0.806 mole) in EtOH (1 L) was heated at reflux for 4 hours, then was cooled to RT. Concentration to dryness gave the title compound (238 g, quantitative).

### b) 6-Methoxy-4-oxo-1,4-dihydro-[1,5]naphthyridine-3-carboxylic acid ethyl ester

Dowtherm A (Fluka, 500 mL) was brought to boiling (250° C.) in a 2 L 3-neck flask fitted with a still-head and a reflux condenser. 2-[(6-Methoxypyridin-3-ylamino)methylene]malonic acid diethyl ester (100 g, 0.34 mole) was added portion-wise over 5 min. The solution was heated at reflux for an additional 15 min, allowing some solvent to distil over. The resulting solution was cooled to RT and diluted with hexanes (750 mL). The mixture was cooled in ice for 1 hr, then the brown solid was filtered off, washed with hexanes, and dried under vacuum to afford the title compound (61.72 g, 73%).

### c) 4-Bromo-6-methoxy-[1,5]naphthyridine-3-carboxylic acid ethyl ester

A suspension of 6-methoxy-4-oxo-1,4-dihydro-[1,5]naphthyridine-3-carboxylic acid ethyl ester (74.57 g, 300 mmole) in dry DMF (260 mL) under argon was stirred efficiently* in a water bath (to maintain approximately RT—may need slight ice-cooling on a large scale). Phosphorus tribromide (30.0 mL, 316 mmole) was added dropwise over 15 min and stirring was continued for an additional 30 min. Water (1 L) was added, followed by saturated sodium carbonate solution to pH 7. The solid was collected by suction filtration, washed with water and dried under vacuum over phosphorus pentoxide to give the title compound (83.56 g, 90%).

### d) 4-Bromo-6-methoxy-[1,5]naphthyridine-3-carboxylic acid

2 N NaOH (300 mL, 600 mmole) was added dropwise over 30 min to a stirred solution of 4-bromo-6-methoxy-[1,5]naphthyridine-3-carboxylic acid ethyl ester (83.56 g, 268 mmole) in THF (835 mL). Stirring was continued overnight, at which time LC/MS showed that the saponification was complete. 2 N HCl was added to pH 6 and the THF was removed in vacuo. 2 N HCl was added to pH 2, then water (250 mL) was added, and the mixture was cooled thoroughly in ice. The solid was collected by suction filtration, washed with water and dried (first using a rotary evaporator at 50° C. and then under high vacuum at 50° C. overnight) to give the title compound (76.7 g, slightly over quantitative). This material was used without further purification.

### (e) 4-Chloro-6-(methyloxy)-1,5-naphthyridine-3-carboxamide

To a solution of 4-Bromo-6-methoxy-[1,5]naphthyridine-3-carboxylic acid ethyl ester (840 mg, 3.0 mmol) in toluene (10 mL) was added thionyl chloride (3 mL) as one portion under N2 protection. After refluxing at 100° C. for 2 h, the mixture was concentrated and azeotropically dried with toluene to afford a yellow solid, which was dissolved in anhydrous DCM (3 mL). The resulting solution was cooled down to 0° C. and treated with NH₃ solution (5 mL, 50% in water). After stirring at 0° C. for 30 min, the reaction mixture was warmed up to 25° C. and stirred for 12 h. DCM was removed, and the solid was collected by suction filtration, washed with water and dried under vacuum over phosphorus pentoxide to give the title compound (648 mg, 91%).

### (f) 4-Chloro-6-(methyloxy)-1,5-naphthyridine-3-carbonitrile

To a solution of 4-chloro-6-(methyloxy)-1,5-naphthyridine-3-carboxamide (647 mg, 2.7 mmol) in anhydrous DCM (2 mL) with triethylamine (2 mL) at 0° C. was added trifluoroacetic anhydride (1 mL) slowly. The resulting solution was warmed up to 25° C. and stirred for 1 h. The mixture was partitioned between CHCl₃ and H₂O. The aqueous layer was extracted several times with CHCl₃. The organic fractions were combined, concentrated and purified with column chromatography (silica, 0-25% ethyl acetate/hexane) affording the title compound as an off-white solid (540 mg, 91%): LC/MS (ES) m/e 220 (M+H)⁺.

### (g) 4-Ethenyl-6-(methyloxy)-1,5-naphthyridine-3-carbonitrile

To a solution of 4-chloro-6-(methyloxy)-1,5-naphthyridine-3-carbonitrile (280 mg, 1.28 mmol), potassium carbonate (885 mg, 6.4 mmole), tetrakis-triphenylphosphine (30 mg, 0.026 mmole) in DME/H₂O (20 mL, 3:1) was added 2,4,6-trivinylcycloborane-pyridine complex (154 mg, 0.64 mmole). After stirring for 1 h at 90° C., another batch of tetrakis-triphenylphosphine (30 mg, 0.026 mmol) was added. After refluxing for another 1.5 h, the mixture contents were cooled down to room temperature and extracted with diethyl ether. The ether fractions were combined, concentrated and purified by column chromatography (silica, 0-10% ethyl acetate in hexane) to give the title compound as a light yellow solid (176 mg, 65%): LC/MS (ES) m/e 212 (M+H)⁺.

### Preparation 13

### Preparation of 7-formyl-2,3-dihydro-1,4-benzodioxin-5-carbonitrile

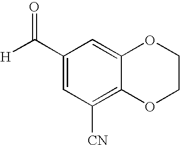

### (a) 3-bromo-4-hydroxy-5-methoxybenzaldehyde

To a solution of vanillin (30.40 g, 0.20 mol) in glacial acetic acid (200 mL) was added bromine (46.79 g, 0.29 mol) in glacial acetic acid (20 mL) at 10° C. over a period of 1 h. Additional acetic acid (100 mL) was added to the thickening mixture and the reaction was stirred for 24 h at ambient temperature. The reaction was diluted with ice/water (300 mL) and the precipitate was filtered and washed well with water. A light beige solid (40.69 g, 89%) was obtained after vacuum drying: MS (ES) m/z 230.0. (M+H)⁺.

### (b) 3-bromo-4,5-dihydroxybenzaldehyde

To a solution of the compound of 3-bromo-4-hydroxy-5-methoxybenzaldehyde (12.1 g, 0.52 mol) in CH₂Cl₂ (200 mL) was added a solution of boron tribromide (115 mL, 1.15 mmol, 1.0 M in DCM) at 0° C. The reaction was stirred at 0° for 20 min, then at ambient temperature for 2.5 h. The reaction was then cooled to 0° C., and quenched by the slow addition of methanol. The solvents were removed under reduced pressure and the trimethyl borate was removed by azeotroping with methanol. Vacuum drying yielded a dark green-brown solid (11.51 g, 100%) which was used without further purification: MS (ES) m/z 217.2. (M+H)⁺.

### (c) 8-bromo-2,3-dihydro-1,4-benzodioxin-6-carbaldehyde

To a solution of 3-bromo-4,5-dihydroxybenzaldehyde (11.5 g, 0.52 mol) in DMF (220 mL) was added cesium carbonate (50.7 g, 1.56 mol). The mixture was stirred at ambient temperature for 30 min, then 1,2-dibromoethane (12.76 g, 0.68 mol) was added. After heating at 800 for 4 h, the DMF was removed under reduced pressure. The residue was partitioned between water and ethyl acetate, and the organic layer washed with brine and dried (MgSO₄). The crude product was purified by flash column chromatography (silica gel, 4:1 hexane:ethyl acetate) to give an off-white solid (9.57 g, 75%): MS (ES) m/z 243.2 (M+H)⁺.

### (d) 7-formyl-2,3-dihydro-1,4-benzodioxin-5-carbonitrile

To a solution of 8-bromo-2,3-dihydro-1,4-benzodioxin-6-carbaldehyde (4.6 g, 18.9 mmol) in DMA (45 mL) was added CuCN (1.82 g, 20.26 mmol). After refluxing for 4 h, the solution was concentrated and the resulting residue was partitioned between ethyl acetate and water. The aqueous layer was extracted several times with ethyl acetate. The organic fractions were combined, washed with brine, concentrated and purified by column chromatography (silica, 15%-30% ethyl acetate in hexane) providing the title compound as an off-white-solid (2.95 g, 82%): LC/MS (ES) m/z 190 (M+H)⁺.

### Preparation 14

### Preparation of 5-formyl-2-3-dihydro-1-benzofuran-7-carbonitrile

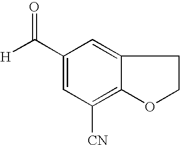

### (a) 7-bromo-2,3-dihydro-1-benzofuran-5-carbaldehyde

To a solution of 2,3-dihydro-1-benzofuran-5-carbaldehyde (1 g, 6.75 mmol) in acetic acid (8 mL) was added sodium acetate (664 mg, 8.1 mmol) followed by bromine (0.7 mL, 13.5 mmol). After stirring at room temperature for 2 h, the mixture was diluted with the aqueous solution of NaHCO₃. The aqueous solution was extracted several times with ethyl acetate. The organic fractions were combined, washed with saturated NaHCO₃, brine and then concentrated to afford the desired compound (1.4 g, 91%) which was used without further purification: LC/MS (ES) m/z 228 (M+H)⁺.

### (b) 5-formyl-2,3-dihydro-1-benzofuran-7-carbonitrile

To a solution of 7-bromo-2,3-dihydro-1-benzofuran-5-carbaldehyde (1.4 g, 6.1 mmol) in DMA (10 mL) was added CuCN (548 mg, 6.1 mmol). After refluxing for 13 h, the solution was concentrated and the resulted residue was partitioned between ethyl acetate and water. The aqueous layer was extracted several times with ethyl acetate. The organic fractions were combined, washed with brine, concentrated and purified by column chromatography (silica, 15%-30% ethyl acetate in hexane) providing the title compound as an off-white-solid (700 mg, 66%): LC/MS (ES) m/z 174 (M+H)⁺.

### Preparation 15

### Preparation of 8-fluoro-3-oxo-3,4-dihydro-2H-1,4-benzoxazine-6-carbaldehyde

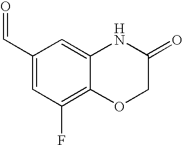

### (a) 6-Bromo-8-fluoro-2H-1,4-benzoxazin-3(4H)-one

A solution of 2-amino-4-bromo-6-fluorophenol (9.1 g) in chloroform (400 mL) was treated with sodium bicarbonate (18.5 g) and benzyltriethylammonium chloride (10.0 g) and cooled in an ice-bath. Chloroacetyl chloride (4.21 mL) was added and the mixture was stirred cold for 1 hour, then heated under reflux for 24 hours. It was cooled, diluted with sodium carbonate solution, stirred, and filtered. The solid washed with water, dichloromethane, then triturated with methanol, and then water, to give a solid (6.3 g; 63%).

LC/MS (−ve ion electrospray): m/z 2441246 (M−H)⁻.

### (b) 6-Ethenyl-8-fluoro-2H-1,4-benzoxazin-3(4h)-one

The bromo-benzooxazinone (a) (6.3 g) in dimethoxyethane (300 mL) was degassed with argon and tetrakis(triphenylphosphine)palladium(0) (1.85 g) added and the solution was further degassed for 20 minutes. Vinylboroxane:pyridine (3.0 g), anhydrous potassium carbonate (3.54 g) and water (75 mL) were added and the mixture was heated at 100° C. overnight. It was evaporated, the solid collected and washed with water, dichloromethane and dried to give a solid (4.4 g; 89%)

LC/MS (−ve ion electrospray): m/z 192 (M−H)⁻.

### (c) 8-Fluoro-3-oxo-3,4-dihydro-2H-1,4-benzoxazine-6-carbaldehyde

The benzooxazinone (b) (4.0 g) in dioxane (150 mL) and water (60 mL) was treated with osmium tetroxide (60 drops of a 4% solution in water) and sodium periodate (9.5 g) and stirred at room temperature for 5 hours. The mixture was diluted with aqueous Na₂SO₃ and ethyl acetate. The organic phase washed with water, brine, dried (sodium sulfate), and evaporated. The solid was triturated with methanol and collected (1.8 g).

LC/MS (−ve ion electrospray): m/z 194 (M−H)⁻.

### EXAMPLE 1

### Preparation of 6-({[(1-{2-[6-(methyloxy)-1,5-naphthyridin-4-yl]-ethyl}-3-piperidinyl)methyl]amino}methyl)-2H-pyrido[3,2-b][1,4]thiazin-3(4H)-one

### (a) 2,2,2-trifluoro-N-(3-piperidinylmethyl)acetamide

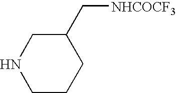

To a solution of 1,1-dimethylethyl 3-(aminomethyl)-1-piperidinecarboxylate (500 mg, 2.3 mmol) in DCM (0.93 mL) at 25° C. were added pyridine (0.57 mL, 7.0 mmol) followed by a solution of TFAA (0.725 mL, 5.13 mmol) in DCM (0.93 mL) dropwise. After 2 h, the reaction was quenched with ice water and extracted several times with DCM. The combined organic fractions were washed with NaOH solution, dried over Na₂SO₄ and concentrated.

The crude residue was dissolved in TFA (12.9 mL) and stirred at 25° C. After 12 h, the solution was concentrated yielding the trifluoroacetyl salt as an orange solid, that was used without further purification: LC/MS (ES) m/e 211 (M+H)⁺.

### (b) 2,2,2-trifluoro-N-[(1-{2-[6-(methyloxy)-1,5-naphthyridin-4-yl]ethyl}-3-piperidinyl)methyl]acetamide

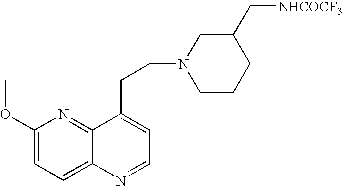

A solution the trifluoroacetyl salt of 2,2,2-trifluoro-N-(3-piperidinylmethyl)acetamide (7.0 g, 21.6 mmol) in DMF (60 ml) at 25° C. was added DIPEA (19 mL, 0.108) followed by 8-ethenyl-2-(methyloxy)-1,5-naphthyridine (3.6 g, 19.5 mmol). After 12 h. at 90° C., the solution was concentrated and the residue purified via column chromatography (silica, 3% MeOH in DCM (1% NH4OH)) yielding the title compound as an orange oil (3.3 g, 43%):

LC/MS (ES) m/e 397 (M+H)⁺.

### (c) [(1-{2-[6-(methyloxy)-1,5-naphthyridin-4-yl]ethyl}-3-piperidinyl)methyl]amine

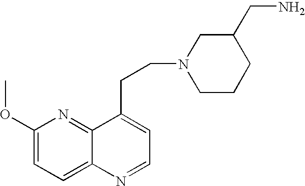

To a solution of 2,2,2-trifluoro-N-[(1-{2-[6-(methyloxy)-1,5-naphthyridin-4-yl]ethyl}-3-piperidinyl)methyl]acetamide (3.3 g, 8.33 mmol) in MeOH:H₂O (115 mL, 1.5:1) was added K₂CO₃ (5.76 g, 41.68 mmol). After 12 h at 25° C., the reaction was concentrated under reduced pressure and purified via column chromatography (silica, 5% MeOH in DCM (2% NH₄OH)) affording the title compound as a yellow oil (1.2 g, 48%): LC/MS (ES) m/e 301 (M+H)⁺.

### (d) 6-({[(1-{2-[6-(methyloxy)-1,5-naphthyridin-4-yl]ethyl}-3-piperidinyl)methyl]amino}methyl)-2H-pyrido[3,2-b][1,4]thiazin-3(4H)-one

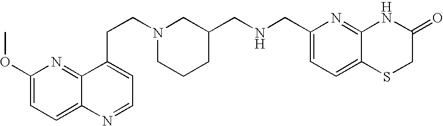

To a solution of [(1-{2-[6-(methyloxy)-1,5-naphthyridin-4-yl]ethyl}-3-piperidinyl)methyl]amine (228 mg, 0.759 mmol) in EtOH:DCM (8 mL, 1:1) were added Na₂SO₄ followed by 3-oxo-3,4-dihydro-2H-pyrido[3,2-b][1,4]thiazine-6-carbaldehyde (122 mg, 0.759 mmol). After 12 h at 25° C., NaBH₄ (34 mg, 0.91 mmol) was added. After an additional 30 min., the reaction was concentrated and the residue was partitioned between DCM/H₂O. The combined organic fractions were dried over MgSO₄, concentrated and purified via column chromatography (silica, 4% MeOH in DCM (1% NH₄OH)) yielding the title compound (254 mg, 70%) as a yellow solid: LC/MS (ES) m/e 479 (M+H)⁺; ¹H NMR (CDCl₃, 400 Hz) δ 8.67 (d, J=4.4 Hz, 1H), 8.18 (d, J=9.0 Hz, 1H), 8.00 (bs, 1H), 7.58 (d, J=7.8 Hz, 1H), 7.44 (bs, 1H), 7.12 (d, J=9.0 Hz, 1H), 6.97 (d, J=7.9 Hz, 1H), 4.08 (s, 3H), 3.80 (s, 2H), 3.48 (s, 2H), 3.45-3.48 (m, 3H), 3.19-3.25 (m, 1H), 2.82-2.99 (m, 1H), 2.45-2.48 (m, 2H), 2.25-2.29 (m, 1H), 1.92-1.96 (m, 2H), 1.45-1.75 (m, 6H).

This material, as a solution in MeOH, was treated with an excess of 4M HCl in dioxane and evaporated to dryness to provide the dihydrochloride salt of the title compound.

### EXAMPLE 2

### Preparation of N-[(1-{2-[6-(methyloxy)-1,5-naphthyridin-4-yl]ethyl}-3-piperidinyl)methyl]-3-oxo-3,4-dihydro-2H-pyrido[3,2-b][1,4]thiazine-6-sulfonamide

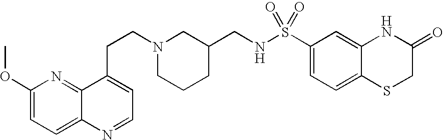

To a solution of [(1-{2-[6-(methyloxy)-1,5-naphthyridin-4-yl]ethyl}-3-piperidinyl)methyl]amine (172 mg, 0.57 mmol) in DCM (10 mL) were added DIPEA (150 mL, 0.86 mmol) and 3-oxo-3,4-dihydro-2H-pyrido[3,2-b][1,4]thiazine-6-sulfonyl chloride (142 mg, 1.22 mmol). After 12 h at room temperature, the solution was concentrated and the residue was purified by column chromatography (silica, 4% MeOH in DCM (1% NH₄OH)) affording the title compound (246 mg, 81%) as an off-white solid: LC/MS (ES) m/e 529 (M+H)⁺; ¹H NMR (CDCl₃, 400 MHz) δ 8.66 (d, J=4.5 Hz, 1H), 8.40 (bs, 1H), 8.17 (d, J=9.0 Hz, 1H), 7.45 (bs, 2H), 7.39-7.41 (m, 2H), 7.10 (d, J=9.0 Hz, 1H), 4.90 (bs, 1H), 4.06 (s, 3H), 3.48 (s, 2H), 3.39-3.41 (m, 2H), 3.14-3.18 (m, 2H), 2.86-2.88 (m, 3H), 2.12-2.15 (m, 2H), 1.70-1.73 (m, 2H), 1.44-1.54 (m, 4H).

This material, as a solution in MeOH, was treated with an excess of 4M HCl in dioxane and evaporated to dryness to provide the dihydrochloride salt of the title compound.

### EXAMPLE 3

### Preparation of N-[(1-{2-[6-(methyloxy)-1,5-naphthyridin-4-yl]ethyl}-3-piperidinyl)methyl]-3-oxo-3,4-dihydro-2H-pyrido[3,2-b][1,4]thiazine-6-carboxamide

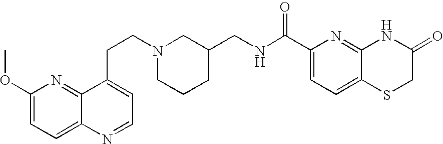

To a solution of [(1-{2-[6-(methyloxy)-1,5-naphthyridin-4-yl]ethyl}-3-piperidinyl)methyl]amine (215 mg, 0.716 mmol) in DCM:DMF (8 mL, 7:1) were added 3-oxo-3,4-dihydro-2H-pyrido[3,2-b][1,4]thiazine-6-carboxylic acid (181 mg, 0.858 mmol) and 1-(3-Dimethylaminopropyl)-3-ethylcarbodimide (133 mg, 0.858 mmol). After 12 h at room temperature, the solution was concentrated and the residue was purified by column chromatography (silica, 1% MeOH in DCM (1% NH₄OH)) affording the title compound as a yellow solid (90 mg, 26%): LC/MS (ES) m/e 493 (M+H)⁺; ¹H NMR (CD₃OD, 400 MHz) δ 8.62 (d, J=4.6 Hz, 1H), 8.19 (d, J=9.1 Hz, 1H), 7.91 (d, J=7.9 Hz, 1H), 7.73 (d, J=7.9 Hz, 1H), 7.59 (d, J=4.6 Hz, 1H), 7.23 (d, J=9.1 Hz, 1H), 4.11 (s, 3H), 3.62 (s, 2H), 3.45-3.49 (m, 2H), 3.34-3.36 (m, 1H), 3.17-3.20 (m, 2H), 2.83-2.87 (m, 2H), 2.22-2.30 (m, 2H), 1.81-1.84 (m, 2H), 1.70-1.73 (m, 2H), 1.40-1.43 (m, 2H).

This material, as a solution in MeOH, was treated with an excess of 4M HCl in dioxane and evaporated to dryness to provide the dihydrochloride salt of the title compound.

### EXAMPLE 4

### Preparation of 6-({[(4-{2-[6-(methyloxy)-1,5-naphthyridin-4-yl]ethyl}-2-morpholinyl)methyl]amino}methyl)-2H-pyrido[3,2-b][1,4]oxazin-3(4H)-one

### (a) 1,1-dimethylethyl {[4-(phenylmethyl)-2-morpholinyl]methyl}carbamate

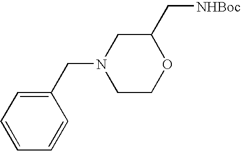

To a solution of 1-[4-(phenylmethyl)-2-morpholinyl]methanamine (4.1 g, 20.1 mmol) in CH₃CN (100 mL) at 25° C. was added Boc₂O (7 mL, 30.15 mmol). After 12 h, the mixture was concentrated and purified via column chromatography (silica, 1% MeOH in DCM (1% NH₄OH)) yielding the title compound (6.1 g, quant.) as a white solid: LC/MS (ES) m/e 306 (M+H)⁺.

### (b) 1,1-dimethylethyl(2-morpholinylmethyl)carbamate

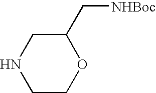

To a solution of 1,1-dimethylethyl {[4-(phenylmethyl)-2-morpholinyl]methyl}carbamate (2.6 g, 8.5 mmol) in EtOH (85 mL) at 25° C. was added 10% Pd/C (780 mg, 30 wt. %). The suspension was hydrogenated at 50 psi using a Parr Shaker. After 12 h, the mixture was filtered through Celite® and the pad washed several times with MeOH. The filtrate was concentrated to afford the title compound (1.7 g, 92%) as a white solid, which was used without further purification: LC/MS (ES) m/e 217 (M+H)⁺.

### (c) 1,1-dimethylethyl[(4-{2-[6-(methyloxy)-1,5-naphthyridin-4-yl]ethyl}-2-morpholinyl)methyl]carbamate

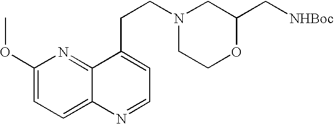

1,1-dimethylethyl(2-morpholinylmethyl)carbamate (1.7 g, 7.87 mmol) and 8-ethenyl-2-(methyloxy)-1,5-naphthyridine (1.3 g, 7.15 mmol) were mixed in DMF (14 mL) and heated at 90° C. over 12 h. The solution was then concentrated and the residue was purified via column chromatography (silica, 3% MeOH in DCM) yielding the title compound (950 mg, 35%) as a white foam; LC/MS (ES) m/e 403 (M+H)⁺.

### (d) [(4-{2-[6-(methyloxy)-1,5-naphthyridin-4-yl]ethyl}-2-morpholinyl)methyl]amine

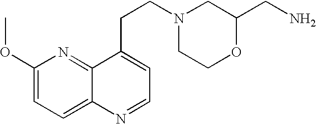

To a solution of 1,1-dimethylethyl[(4-{2-[6-(methyloxy)-1,5-naphthyridin-4-yl]ethyl}-2-morpholinyl)methyl]carbamate (950 mg, 2.36 mmol) in DCM (24 mL) at 25° C. was added dropwise a solution of HCl in dioxane (3 mL, 12 mmol, 4M HCl in dioxane). After 12 h, the reaction was concentrated to afford the HCl salt of the title compound (546 mg, 76%) as a yellow foam, which was used without further purification: LC/MS (ES) m/e 303 (M+H)⁺.

### (e) 6-({[(4-{2-[6-(methyloxy)-1,5-naphthyridin-4-yl]ethyl}-2-morpholinyl)methyl]amino}methyl)-2H-pyrido[3,2-b][1,4]thiazin-3(4H)-one

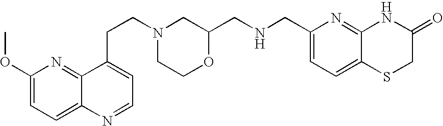

To a solution of the HCl salt of [(4-{2-[6-(methyloxy)-1,5-naphthyridin-4-yl]ethyl}-2-morpholinyl)methyl]amine (200 mg, 0.552 mmol) in EtOH:DCM (6 mL, 1:1) were added Na₂SO₄ (78 mg, 0.55 mmol) followed by DIPEA (0.481 mL, 2.76 mmol) and 3-oxo-3,4-dihydro-2H-pyrido[3,2-b][1,4]thiazine-6-carbaldehyde (89 mg, 0.553 mmol). After 12 h at 25° C., NaBH₄ (25 mg, 0.663 mmol) was added. After an additional 1 h, the reaction was concentrated and the residue was partitioned between DCM-H₂O. The aqueous phase was extracted several times with DCM and the combined organic fractions were dried over MgSO₄, concentrated and purified via column chromatography (silica, 4% MeOH in DCM (1% NH₄OH)) yielding the title compound (150 mg, 57%) as a yellow foam: LC/MS (ES) m/e 481 (M+H)⁺; ¹H NMR (CDCl₃, 400 Hz) δ 8.70 (d, J=4.4 Hz, 1H), 8.25 (bs, 1H), 8.22 (d, J=9.0 Hz, 1H), 7.62 (d, J=7.8 Hz, 1H), 7.46 (d, J=4.3 Hz, 1H), 7.14 (d, J=9.0 Hz, 1H), 7.01 (d, J=7.7 Hz, 1H), 4.09 (s, 3H), 3.82-3.86 (m, 1H), 3.66-3.76 (m, 4H), 3.51 (s, 2H), 3.26-3.31 (m, 2H), 2.80-2.83 (m, 2H), 2.72-2.75 (m, 3H), 2.64-2.67 (m, 2H), 2.22-2.25 (m, 1H), 1.99-2.01 (m, 1H).

This material, as a solution in MeOH, was treated with an excess of 4M HCl in dioxane and evaporated to dryness to provide the dihydrochloride salt of the title compound.

### EXAMPLE 5

### Preparation of N-[(4-{2-[6-(methyloxy)-1,5-naphthyridin-4-yl]ethyl}-2-morpholinyl)methyl]-3-oxo-3,4-dihydro-2H-pyrido[3,2-b][1,4]thiazine-6-carboxamide

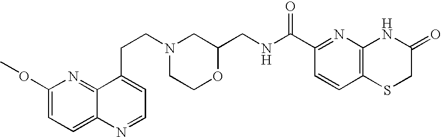

To a solution of the HCl salt of [(4-{2-[6-(methyloxy)-1,5-naphthyridin-4-yl]ethyl}-2-morpholinyl)methyl]amine (250 mg, 0.69 mmol) in DCM/DMF (10 mL, 4:1) were added DIPEA (0.6 mL, 3.5 mmol), 3-oxo-3,4-dihydro-2H-pyrido[3,2-b][1,4]thiazine-6-carboxylic acid (175 mg, 0.824 mmol) and 1-(3-Dimethylaminopropyl)-3-ethylcarbodimide (129 mg, 0.824 mmol). After 12 h at room temperature, the solution was concentrated and purified by column chromatography (silica, 3% MeOH in DCM (1% NH₄OH)) affording the title compound (40 mg, 12%) as a yellow foam: LC/MS (ES) m/e 495 (M+H)⁺; ¹H NMR (CDCl₃, 400 MHz) δ 8.76 (bs, 1H), 8.59 (bs, 1H), 8.12 (d, J=9.0 Hz, 1H), 7.87 (bs, 1H), 7.76 (d, J=7.9 Hz, 1H), 7.68 (d, J=7.9 Hz, 1H), 7.34 (bs, 1H), 7.03 (d, J=9.0 Hz, 1H), 3.98 (s, 3H), 3.84-3.87 (m, 1H), 3.58-3.67 (m, 3H), 3.46 (s, 2H), 3.27-3.43 (m, 3H), 2.89-2.91 (m, 1H), 2.80-2.81 (m, 1H), 2.71-2.79 (m, 2H), 2.20-2.23 (m, 1H), 1.96-2.09 (m, 1H).

This material, as a solution in MeOH, was treated with an excess of 4M HCl in dioxane and evaporated to dryness to provide the dihydrochloride salt of the title compound.

### EXAMPLE 6

### Preparation of N-[(4-{2-[6-(methyloxy)-1,5-naphthyridin-4-yl]ethyl}-2-morpholinyl)methyl]-3-oxo-3,4-dihydro-2H-1,4-benzothiazine-6-sulfonamide

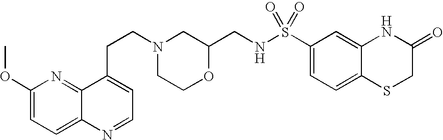

The title compound (80 mg, 44%) was prepared as a yellow solid according to Example 2, except substituting the HCl salt of [(4-{2-[6-(methyloxy)-1,5-naphthyridin-4-yl]ethyl}-2-morpholinyl)methyl]amine (149 mg, 0.41 mmol) for [(1-{2-[6-(methyloxy)-1,5-naphthyridin-4-yl]ethyl}-3-piperidinyl)methyl]amine: LC/MS (ES) m/e 531 (M+H)⁺; ¹H NMR (CD₃OD, 400 MHz) δ 8.63 (d, J=4.6 Hz, 1H), 8.20 (d, J=9.1 Hz, 1H), 7.60 (d, J=4.5 Hz, 1H), 7.51 (d, J=8.1 Hz, 1H), 7.46 (d, J=8.1 Hz, 1H), 7.43 (s, 1H), 7.23 (d, J=9.1 Hz, 1H), 4.12 (s, 3H), 3.82-3.85 (m, 1H), 3.55-3.57 (m 2H), 3.54 (s, 2H), 3.40-3.43 (m, 2H), 2.97-2.99 (m, 3H), 2.84-2.86 (m, 1H), 2.80-2.83 (m, 2H), 2.28-2.31 (m, 1H), 2.01-2.05 (m, 1H).

This material, as a solution in MeOH, was treated with an excess of 4M HCl in dioxane and evaporated to dryness to provide the dihydrochloride salt of the title compound.

### EXAMPLE 7

### Preparation of 6-({[((2R)-4-{2-[6-(methyloxy)-1,5-naphthyridin-4-yl]ethyl}-2-morpholinyl)methyl]amino}methyl)-2H-pyrido[3,2-b][1,4]thiazin-3(4H)-one

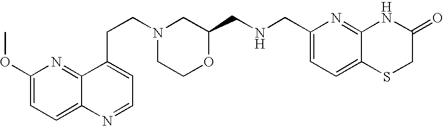

The title compound (257 mg, 68%) was prepared as a yellow foam according to Example 4, except substituting 1,1-dimethylethyl {[(2R)-4-(phenylmethyl)-2-morpholinyl]methyl}carbamate (2.52 g, 8.26 mmol) for the racemic mixture: LC/MS (ES) m/e 481 (M+H)⁺; ¹H NMR (CDCl₃, 400 Hz) δ 8.70 (d, J=4.4 Hz, 1H), 8.25 (bs, 1H), 8.22 (d, J=9.0 Hz, 1H), 7.62 (d, J=7.8 Hz, 1H), 7.46 (d, J=4.3 Hz, 1H), 7.14 (d, J=9.0 Hz, 1H), 7.01 (d, J=7.7 Hz, 1H), 4.09 (s, 3H), 3.82-3.86 (m, 1H), 3.66-3.76 (m, 4H), 3.51 (s, 2H), 3.26-3.31 (m, 2H), 2.80-2.83 (m, 2H), 2.72-2.75 (m, 3H), 2.64-2.67 (m, 2H), 2.22-2.25 (m, 1H), 1.99-2.01 (m, 1H).

This material, as a solution in MeOH, was treated with an excess of 4M HCl in dioxane and evaporated to dryness to provide the dihydrochloride salt of the title compound.

### EXAMPLE 8

### Preparation 6-({[((2S)-4-{2-[6-(methyloxy)-1,5-naphthyridin-4-yl]ethyl}-2-morpholinyl)methyl]amino}methyl)-2H-pyrido[3,2-b][1,4]thiazin-3(4H)-one

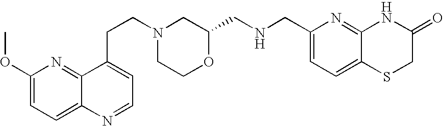

The title compound (316 mg, 63%) was prepared as a yellow foam according to Example 4, except substituting 1,1-dimethylethyl {[(2S)-4-(phenylmethyl)-2-morpholinyl]methyl}carbamate (2.35 g, 7.69 mmol) for the racemic mixture: LC/MS (ES) m/e 481 (M+H)⁺; ¹H NMR (CDCl₃, 400 Hz) δ 8.70 (d, J=4.4 Hz, 1H), 8.25 (bs, 1H), 8.22 (d, J=9.0 Hz, 1H), 7.62 (d, J=7.8 Hz, 1H), 7.46 (d, J=4.3 Hz, 1H), 7.14 (d, J=9.0 Hz, 1H), 7.01 (d, J=7.7 Hz, 1H), 4.09 (s, 3H), 3.82-3.86 (m, 1H), 3.66-3.76 (m, 4H), 3.51 (s, 2H), 3.26-3.31 (m, 2H), 2.80-2.83 (m, 2H), 2.72-2.75 (m, 3H), 2.64-2.67 (m, 2H), 2.22-2.25 (m, 1H), 1.99-2.01 (m, 1H).

This material, as a solution in MeOH, was treated with an excess of 4M HCl in dioxane and evaporated to dryness to provide the dihydrochloride salt of the title compound.

### EXAMPLE 9

### Preparation of 6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]thiazin-3(4H)-one

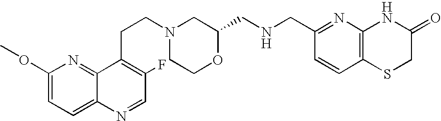

The title compound (88 mg, 52%) was prepared as a yellow foam according to Example 4, except substituting 1,1-dimethylethyl {[(2S)-4-(phenylmethyl)-2-morpholinyl]methyl}carbamate (2.35 g, 7.69 mmol) for the racemic mixture and 8-ethenyl-7-fluoro-2-(methyloxy)-1,5-naphthyridine (185 mg, 0.908 mmol) for 8-ethenyl-2-(methyloxy)-1,5-naphthyridine: LC/MS (ES) m/e 499 (M+H)⁺; ¹H NMR (CD₃OD, 400 MHz) δ 8.66 (s, 1H), 8.21 (d, J=9.0 Hz, 1H), 7.70 (d, J=7.8 Hz, 1H), 7.19 (d, J=9.0 Hz, 1H), 7.01 (d, J=7.8 Hz, 1H), 4.11 (s, 3H), 3.89-3.92 (m, 1H), 3.79-3.81 (m, 2H), 3.64-3.66 (m, 2H), 3.52 (s, 2H), 3.46-3.50 (m, 2H), 2.94-2.97 (m, 2H), 2.80-2.83 (m, 2H), 2.76-2.78 (m, 2H), 2.26-2.28 (m, 1H), 2.03-2.05 (m, 1H).

This material, as a solution in MeOH, was treated with an excess of 4M HCl in dioxane and evaporated to dryness to provide the dihydrochloride salt of the title compound.

### EXAMPLE 10

### Preparation of 6-({[((2R)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one

### (a) 1,1-dimethylethyl[((2R)-4-{(2R)-2-hydroxy-2-[6-(methyloxy)-1,5-naphthyridin-4-yl]ethyl}-2-morpholinyl)methyl]carbamate

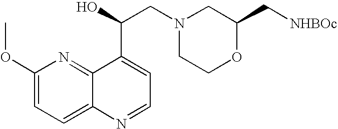

A solution of 2-(methyloxy)-8-(2-oxiranyl)-1,5-naphthyridine (170 mg, 0.842 mmol) and 1,1-dimethylethyl[(2S)-2-morpholinylmethyl]carbamate (200 mg, 0.926 mmol) in DMF (2 mL) were heated to 90° C. After 12 h, the resulting solution was concentrated and purified via column chromatography (silica, 3% MeOH in DCM (1% NH₄OH)) yielding the title compound (203 mg, 52%) as an orange oil: LC/MS (ES) m/e 419 (M+H)⁺.

### (b) 6-({[((2R)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one

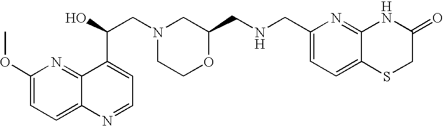

The title compound (145 mg, 60%) was prepared as a yellow solid according to Example 4, except substituting 1,1-dimethylethyl[((2R)-4-{(2R)-2-hydroxy-2-[6-(methyloxy)-1,5-naphthyridin-4-yl]ethyl}-2-morpholinyl)methyl]carbamate (203 mg, 0.485 mmol) for 1,1-dimethylethyl[(4-{2-[6-(methyloxy)-1,5-naphthyridin-4-yl]ethyl}-2-morpholinyl)methyl]carbamate: LC/MS (ES) m/e 497 (M+H)⁺; ¹H NMR (CD₃OD, 400 MHz) δ 8.71 (d, J=4.6 Hz, 1H), 8.19 (d, J=9.1 Hz, 1H), 7.86 (d, J=4.6 Hz, 1H), 7.64 (d, J=7.8 Hz, 1H), 7.20 (d, J=9.1 Hz, 1H), 6.98 (d, J=7.8 Hz, 1H), 5.94 (dd, J=2.3, 9.1 Hz, 1H), 4.05 (s, 3H), 3.89-3.92 (m, 1H), 3.67-3.80 (m, 4H), 3.50 (s, 2H), 3.12-3.15 (m, 1H), 2.81-2.87 (m, 2H), 2.51-2.68 (m, 3H), 2.29-2.35 (m, 1H), 2.03-2.09 (m, 1H).

This material, as a solution in MeOH, was treated with an excess of 4M HCl in dioxane and evaporated to dryness to provide the dihydrochloride salt of the title compound.

### EXAMPLE 11

### Preparation of 6-({[((2S)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one

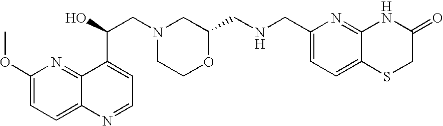

The title compound (89 mg, 43%) was prepared as a yellow solid according to Example 10, except substituting 1,1-dimethylethyl[(2R)-2-morpholinylmethyl]carbamate (200 mg, 0.926 mmol) for 1,1-dimethylethyl[(2S)-2-morpholinylmethyl]carbamate: LC/MS (ES) m/e 497 (M+H)⁺; ¹H NMR (CD₃OD, 400 MHz) δ 8.71 (d, J=4.6 Hz, 1H), 8.19 (d, J=9.1 Hz, 1H), 7.86 (d, J=4.6 Hz, 1H), 7.64 (d, J=7.8 Hz, 1H), 7.20 (d, J=9.1 Hz, 1H), 6.98 (d, J=7.8 Hz, 1H), 5.94 (dd, J=2.3, 9.1 Hz, 1H), 4.05 (s, 3H), 3.89-3.92 (m, 1H), 3.67-3.80 (m, 4H), 3.50 (s, 2H), 3.12-3.15 (m, 1H), 2.81-2.87 (m, 2H), 2.51-2.68 (m, 3H), 2.29-2.35 (m, 1H), 2.03-2.09 (m, 1H).

This material, as a solution in MeOH, was treated with an excess of 4M HCl in dioxane and evaporated to dryness to provide the dihydrochloride salt of the title compound.

### EXAMPLE 12

### Preparation of N-methyl-4-{2-[6-(methyloxy)-1,5-naphthyridin-4-yl]ethyl}-N-[(3-oxo-3,4-dihydro-2H-pyrido[3,2-b][1,4]thiazin-6-yl)methyl]-2-morpholinecarboxamide

### (a) 6-[(methylamino)methyl]-2H-pyrido[3,2-b][1,4]thiazin-3(4H)-one

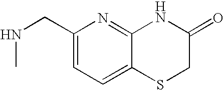

To a solution of 3-oxo-3,4-dihydro-2H-pyrido[3,2-b][1,4]thiazine-6-carbaldehyde (300 mg, 1.54 mmol) in DCM:MeOH (15 mL, 1:1) at 25° C. was added MeNH₂ (0.769 mL, 1.54 mmol, 2M in MeOH). After 12 h, NaBH₄ (70 mg, 1.85 mmol) was added and the solution was stirred an additional 1 h. The reaction mixture was concentrated, then partitioned between DCM-H₂O. The aqueous solution was extracted several times with DCM and the organic fractions were combined, concentrated and purified by column chromatography (silica, 0-3% MeOH in DCM (1% NH₄OH)) affording the title compound (280 mg, 87%) as a white solid: LC/MS (ES) m/e 210 (M+H)⁺.

### (b) 1,1-dimethylethyl 2-({methyl[(3-oxo-3,4-dihydro-2H-pyrido[3,2-b][1,4]thiazin-6-yl)methyl]amino}carbonyl)-4-morpholinecarboxylate

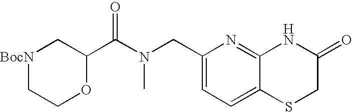

To a solution of 4-{[(1,1-dimethylethyl)oxy]carbonyl}-2-morpholinecarboxylic acid (308 mg, 1.3 mmol) in DCM:DMF (13 ml, 5:1) at 25° C. were added 6-[(methylamino)methyl]-2H-pyrido[3,2-b][1,4]thiazin-3(4H)-one (280 mg, 1.3 mmol), 1-(3-Dimethylaminopropyl)-3-ethylcarbodimide (248 mg, 1.59 mmol) and 1-hydroxybenzotriazole (216 mg, 1.59 mmol). After 12 h, the reaction was concentrated and purified by column chromatography (silica, 0-3% MeOH in DCM (1% NH₄OH)) affording the title compound (282 mg, 50%) as an off-white solid: LC/MS (ES) m/e 423 (M+H)⁺.

### (c) N-methyl-N-[(3-oxo-3,4-dihydro-2H-pyrido[3,2-b][1,4]thiazin-6-yl)methyl]-2-morpholinecarboxamide

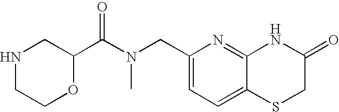

A solution of 1,1-dimethylethyl 2-({methyl[(3-oxo-3,4-dihydro-2H-pyrido[3,2-b][1,4]thiazin-6-yl)methyl]amino}carbonyl)-4-morpholinecarboxylate (282 mg, 0.668 mmol) in MeOH (32 mL) was treated dropwise with a solution of HCl (0.84 mL, 3.34 mmol, 4M solution in dioxane). After 12 h, the solution was concentrated and the crude salt was used without further purification (217 mg, quant.): LC/MS (ES) m/e 323 (M+H)⁺.

### (d) N-methyl-4-{2-[6-(methyloxy)-1,5-naphthyridin-4-yl]ethyl}-N-[(3-oxo-3,4-dihydro-2H-pyrido[3,2-b][1,4]thiazin-6-yl)methyl]-2-morpholinecarboxamide

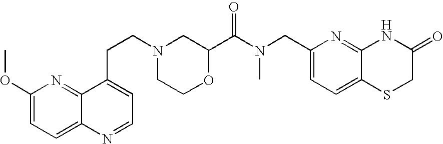

A solution of the HCl salt of N-methyl-N-[(3-oxo-3,4-dihydro-2H-pyrido[3,2-b][1,4]thiazin-6-yl)methyl]-2-morpholinecarboxamide (217 mg, 0.668 mmol), DIPEA (0.349 mL, 2.0 mmol) and 8-ethenyl-2-(methyloxy)-1,5-naphthyridine (113 mg, 0.607 mmol) in DMF (7 mL) were heated to 90° C. After 12 h, the solution was concentrated and purified via column chromatography (silica, 1% MeOH in DCM (1% NH₄OH)) yielding the title compound (280 mg, 83%) as a yellow solid: LC/MS (ES) m/e 509 (M+H)⁺; ¹H NMR (CD₃OD, 400 MHz, doubling observed due to rotamer effect) δ 8.62 (dd (doubling observed), 1H), 8.19 (d, J=9.0 Hz, 1H), 7.71 (dd (doubling observed), 1H), 7.62 (dd (doubling observed), 1H), 7.24 (d, J=9.1 Hz, 1H), 6.95 (dd (doubling observed), 1H), 4.42-4.59 (m, 3H), 4.09 (s, 3H), 3.92-3.97 (m, 1H), 3.70-3.78 (m, 1H), 3.51 (s, 3H), 3.42-3.46 (m, 1H), 3.17 (s, 2H), 3.13-3.16 (m, 1H), 2.88-2.99 (m, 4H), 2.36-2.47 (m, 2H).

This material, as a solution in MeOH, was treated with an excess of 4M HCl in dioxane and evaporated to dryness to provide the dihydrochloride salt of the title compound.

### EXAMPLE 13

### Preparation of 6-({[(4-{2-[3-fluoro-6-(methyloxy)-1,5-naphthyridin-4-yl]ethyl}-2-piperazinyl)methyl]amino}methyl)-2H-pyrido[3,2-b][1,4]thiazin-3(4H)-one

### (a) 1,1-dimethylethyl {[1,4-bis(phenylmethyl)-2-piperazinyl]methyl}carbamate

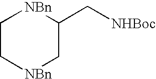

To a solution of 1,4-bis(phenylmethyl)-2-piperazinecarbonitrile (1 g, 3.44 mmol) in THF (34 mL) at 0° C. was added dropwise a solution of LAH (17 mL, 17 mmol, 1.0 M in THF) dropwise. The solution was warmed up to room temperature and stirred for an additional 12 h. The reaction was quenched by dropwise addition of sodium potassium tartrate then extracted several times with DCM. The organic fractions were combined, dried (Na₂SO₄) and concentrated providing the amine as a clear oil, which was used directly.

To the crude amine in THF (34 mL) at 25° C. was added bis(1,1-dimethylethyl) dicarbonate (888 mg, 4.07 mmol) After 12 h., the resulting solution was concentrated and purified via column chromatography (silica, 1% MeOH in DCM (1% NH4OH)) providing the title compound as a clear oil (1.1 g, 82%): LC/MS (ES) m/e 396 (M+H)⁺.

### (b) 1,1-dimethylethyl(2-piperazinylmethyl)carbamate

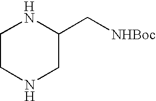

To a solution of 1,1-dimethylethyl {[1,4-bis(phenylmethyl)-2-piperazinyl]methyl}carbamate (1.1 g, 2.78 mmol) in EtOH (85 mL) was added Pd(OH)₂ (660 mg, 30 wt %). The suspension was hydrogenated at 50 psi using a Parr shaker. After 12 h, the suspension was filtered through Celite® and washed several times with MeOH. The filtrate was concentrated to afford the title compound (600 mg, quant.) as a white solid, which was used without further purification; LC/MS (ES) m/e 216 (M+H)⁺.

### (d) 1,1-dimethylethyl[(4-{2-[3-fluoro-6-(methyloxy)-1,5-naphthyridin-4-yl]ethyl}-2-piperazinyl)methyl]carbamate

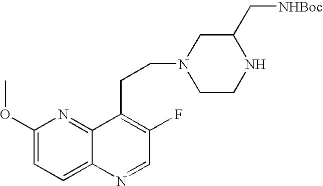

8-ethenyl-7-fluoro-2-(methyloxy)-1,5-naphthyridine (568 mg, 2.78 mmol) and 1,1-dimethylethyl(2-piperazinylmethyl)carbamate (600 mg, 2.78 mmol) were mixed in EtOH (5 mL) and heated at 85° C. over 12 h. The solution was then concentrated and the residue was purified via column chromatography (silica, 1% MeOH in DCM (1% NH4OH)) yielding the title compound (645 mg, 55%) as a white foam: LC/MS (ES) m/e 420 (M+H)⁺.

### (d) [(4-{2-[3-fluoro-6-(methyloxy)-1,5-naphthyridin-4-yl]ethyl}-2-piperazinyl)methyl]amine

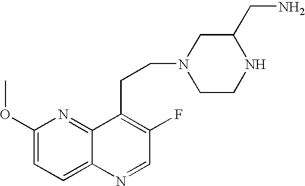

To a solution of 1,1-dimethylethyl[(4-{2-[3-fluoro-6-(methyloxy)-1,5-naphthyridin-4-yl]ethyl}-2-piperazinyl)methyl]carbamate (380 mg, 0.907 mmol) in MeOH (20 mL) at 25° C. was added dropwise an HCl solution (2.3 mL, 9.2 mmol, 4M HCl in dioxane). After 12 h, the reaction was concentrated, re-dissolved in DCM (2 mL) and neutralized with DIPEA (0.5 mL). The resulting solution was then concentrated and washed through a silica pad (5% MeOH in DCM (1% NH₄OH)) to afford the title compound as a yellow oil (220 mg, 76%): LC/MS (ES) m/e 320 (M+H)⁺.

### (e) 6-({[(4-{2-[3-fluoro-6-(methyloxy)-1,5-naphthyridin-4-yl]ethyl}-2-piperazinyl)methyl]amino}methyl)-2H-pyrido[3,2-b][1,4]thiazin-3(4H)-one

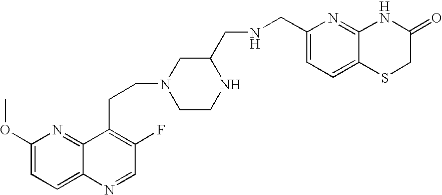

To a solution of [(4-{2-[3-fluoro-6-(methyloxy)-1,5-naphthyridin-4-yl]ethyl}-2-piperazinyl)methyl]amine (100 mg, 0.313 mmol) in DCM:DMF (4 mL, 1:1) were added Na₂SO₄ (67 mg, 0.47 mmol) followed by 3-oxo-3,4-dihydro-2H-pyrido[3,2-b][1,4]thiazine-6-carbaldehyde (61 mg, 0.31 mmol). After 12 h at 25° C., NaBH₄ (24 mg, 0.62 mmol) was added. After an additional 1 h, the reaction was concentrated and the residue was partitioned between DCM-H₂O. The aqueous phase was extracted several times with DCM and the combined organic fractions were dried over Na₂SO₄, concentrated and purified via column chromatography (silica, 2-3% MeOH in DCM (1% NH₄OH)) yielding the title compound (50 mg, 32%) as a yellow solid: LC/MS (ES) m/e 498 (M+H)⁺; ¹H NMR (CD₃OD, 400 Hz) δ 8.64 (s, 1H), 8.20 (d, J=9.1 Hz, 1H), 7.68 (d, J=7.8 Hz, 1H), 7.17 (d, J=9.1 Hz, 1H), 7.02 (d, J=7.8 Hz, 1H), 4.10 (s, 3H), 3.78 (s, 2H), 3.51 (s, 2H), 3.46-3.55 (m, 2H), 2.98-3.06 (m, 3H), 2.78-2.88 (m, 4H), 2.59 (d, J=6.3 Hz, 2H), 2.21-2.26 (m 1H), 1.90-1.95 (m, 1H).

This material, as a solution in MeOH, was treated with an excess of 4M HCl in dioxane and evaporated to dryness to provide the dihydrochloride salt of the title compound.

### EXAMPLE 14

### Preparation of 6-{[7-{2-[3-fluoro-6-(methyloxy)-1,5-naphthyridin-4-yl]ethyl}-3-oxohexahydroimidazo[1,5-a]pyrazin-2(3H)-yl]methyl}-2H-pyrido[3,2-b][1,4]thiazin-3(4H)-one

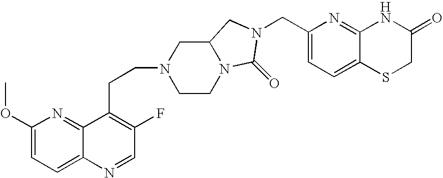

To a solution of 6-({[(4-{2-[3-fluoro-6-(methyloxy)-1,5-naphthyridin-4-yl]ethyl}-2-piperazinyl)methyl]amino}methyl)-2H-pyrido[3,2-b][1,4]thiazin-3(4H)-one (68 mg, 0.137 mmol) (prepared according to example 13) and triethylamine (0.05 mL, 0.342 mmol) in DCM (2 mL) at 0° C. was added dropwise a solution of phosgene (0.072 mL, 0.137 mmol, 20% in toluene). The reaction stirred for 3 h at this temperature and was concentrated and purified by column chromatography (silica, 2% MeOH in DCM (1% NH₄OH)) yielding a brownish oil, which was purified further using a prep silica plate (3% MeOH in DCM (1% NH₄OH)) affording the title compound as an orange solid (11 mg, 15%): LC/MS (ES) m/e 524 (M+H)⁺; ¹H NMR (CD₃OD, 400 Hz) δ 8.54 (s, 1H), 8.09 (d, J=9.1 He, 1H), 7.59 (d, J=7.8 Hz, 1H), 7.07 (d, J=9.1 Hz, 1H), 6.84 (d, J=7.8 Hz, 1H), 4.30 (s, 2H), 3.98 (s, 3H), 3.68-3.71 (m, 1H), 3.58-3.67 (m, 1H), 3.33-3.43 (m, 5H), 2.88-2.95 (m, 4H), 2.72-2.76 (m, 2H), 1.95-2.08 (m, 2H).

This material, as a solution in MeOH, was treated with an excess of 4M HCl in dioxane and evaporated to dryness to provide the dihydrochloride salt of the title compound.

### EXAMPLE 15

### Preparation of 6-{[({(2S)-4-[2-(3-fluoro-6-methoxy-1,5-naphthyridin-4-yl)ethyl]morpholin-2-yl}methyl)amino]methyl}-2H-pyrido[3,2-b][1,4]oxazin-3(4H)-one

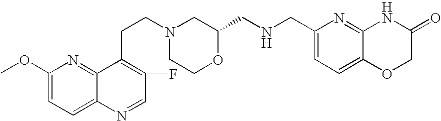

To a solution of the free base of ({4-[2-(3-fluoro-6-methoxy-1,5-naphthyridin-4-yl)ethyl]morpholin-2-yl}methyl)amine (121 mg, 0.378 mmol) in EtOH:DCM (1:10) were added Na₂SO₄ (289 mg, 2.03 mmol) followed by 3-oxo-3,4-dihydro-2H-pyrido[3,2-b][1,4]oxazine-6-carbaldehyde (66.7 mg, 0.374 mmol). After 12 h at 25° C., NaB(0₂CCH₃) ₃H (127 mg, 0.600 mmol) was added. After an additional 1 h, the reaction was filtered and concentrated and the residue purified by chromatography (silica, 10% MeOH in DCM (1% NH₄OH)) yielding the title compound (182 mg, 99%) as a yellow foam: LCMS (+ve ion electrospray) m/z 483 (M+H)⁺; ¹H NMR (CHCl₃, 400 MHz) δ 8.59 (s, 1H), 8.17 (d, J=9.06 Hz, 1H), 7.16 (d, J=8.03 Hz, 1H), 7.03 (d, J=9.05 Hz, 1H), 6.91 (d, J=8.07 Hz, 1H), 4.58 (s, 2H), 4.02 (s, 3H), 3.96-3.72 (m, 4H), 3.68-3.62 (m, 1H), 3.39-3.29 (m, 2H), 2.95-2.85 (m, 2H), 2.77-2.68 (m, 4H), 2.26 (m, 1H), 2.07-1.95 (m, 3H).

This material, as a solution in MeOH, was treated with excess of 2M HCl in diethyl ether and evaporated to dryness to provide the dihydrochloride salt of the title compound.

### EXAMPLE 16

### 6-methoxy-4-{2-[(2S)-2-({[(3-oxo-3,4-dihydro-2H-pyrido[3,2-b][1,4]oxazin-6-yl)methyl]amino}methyl)morpholin-4-yl]ethyl}-1,5-naphthyridine-3-carbonitrile

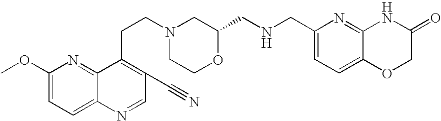

The title compound (78 mg, 38%) was prepared as a yellow solid according to Example 4, except substituting 4-{2-[(2S)-2-(aminomethyl)morpholin-4-yl]ethyl}-6-methoxy-1,5-naphthyridine-3-carbonitrile (166 mg, 0.415 mmol) for ({4-[2-(3-fluoro-6-methoxy-1,5-naphthyridin-4-yl)ethyl]morpholin-2-yl}methyl)amine and 3-oxo-3,4-dihydro-2H-pyrido[3,2-b][1,4]oxazine-6-carbaldehyde (74 mg, 0.415 mmol) for 3-oxo-3,4-dihydro-2H-pyrido[3,2-b][1,4]thiazine-6-carbaldehyde and using NaHCO₃ (350 mg, 4.17 mmol) for DIEA: LC/MS (+ve ion electrospray) m/z 490 (M+H)⁺; ¹H NMR (CHCl₃, 400 MHz) δ 8.85 (s, 1H), 8.25 (d, J=9.08 Hz, 1H), 7.26-7.21 (m, 2H), 6.96 (d, J=8.06 Hz, 1H), 4.65 (s, 2H), 4.09 (s, 3H), 4.00-3.68 (m, 4H), 3.72-3.63 (m, 1H), 3.65-3.59 (m, 2H), 2.98-2.70 (m, 5H), 2.39-2.33 (m, 1H), 2.10-2.02 (m, 4H).

This material, as a solution in MeOH, was treated with excess of 2M HCl in diethyl ether and evaporated to dryness to provide the dihydrochloride salt of the title compound.

### EXAMPLE 17

### 6-methoxy-4-{2-[(2S)-2-({[(3-oxo-3,4-dihydro-2H-Pyrido[3,2-b][1,4]thiazin-6-yl)methyl]amino}methyl)morpholin-4-yl]ethyl}-1,5-naphthyridine-3-carbonitrile

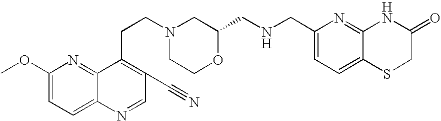

The title compound (95 mg, 50%) was prepared as a yellow solid according to Example 16, except substituting 3-oxo-3,4-dihydro-2H-pyrido[3,2-b][1,4]thiazine-6-carbaldehyde for 3-oxo-3,4-dihydro-2H-pyrido[3,2-b][1,4]oxazine-6-carbaldehyde. This aldehyde (73 mg, 0.373 mmol) was added to 4-{2-[(2S)-2-(aminomethyl)morpholin-4-yl]ethyl}-6-methoxy-1,5-naphthyridine-3-carbonitrile (151 mg, 0.377 mmol) followed by NaHCO₃ (330 mg, 3.93 mmol): LC/MS (+ve ion electrospray) m/z 506 (M+H)⁺; ¹H NMR (CHCl₃, 400 MHz) δ 8.85 (s, 1H), 8.25 (d, J=9.08 Hz, 1H), 7.59 (d, J=7.81 Hz, 1H), 7.24 (d, J=9.08 Hz, 1H), 6.98 (d, J=7.83 Hz, 1H), 4.09 (s, 3H), 3.95-3.90 (m, 3H), 3.89-3.85 (m, 1H), 3.71-3.63 (m, 1H), 3.62-3.56 (m, 1H), 3.48 (s, 2H), 2.93-2.75 (m, 6H), 2.38-2.33 (m, 1H), 2.11-2.06 (m, 1H), 2.02 (s, 3H).

This material, as a solution in MeOH, was treated with excess of 2M HCl in diethyl ether and evaporated to dryness to provide the dihydrochloride salt of the title compound.

### EXAMPLE 18

### 6-({[((2S)-4-{2-[3-fluoro-6-(methyloxy)-4-quinolinyl]ethyl}-2-morpholinyl)methyl]amino}methyl)-2H-pyrido[3,2-b][1,4]thiazin-3(4H)-one

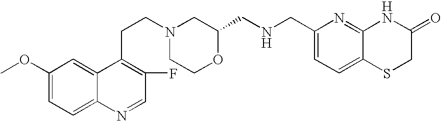

The title compound (78 mg, 48%) was prepared as a yellow solid according to Example 16, except substituting [((2S)-4-{2-[3-fluoro-6-(methyloxy)-4-quinolinyl]ethyl}-2-morpholinyl)methyl]amine (127 mg, 0.324 mmol) for 4-{2-[(2S)-2-(aminomethyl)morpholin-4-yl]ethyl}-6-methoxy-1,5-naphthyridine-3-carbonitrile and 3-oxo-3,4-dihydro-2H-pyrido[3,2-b][1,4]thiazine-6-carbaldehyde (61 mg, 0.314 mmol) for 3-oxo-3,4-dihydro-2H-pyrido[3,2-b][1,4]oxazine-6-carbaldehyde: LC/MS (+ve ion electrospray) m/z 498 (M+H)⁺;

¹H NMR (CHCl₃, 400 MHz) δ 9.68 (br s, 1H), 8.59 (s, 1H), 8.01 (d, J=9.22 Hz, 1H), 7.56 (d, J=7.81 Hz, 1H), 7.32 (d, J=9.20 Hz, 1H), 7.18 (s, 1H), 6.96 (d, J=7.83 Hz, 1H), 6.29 (br s, 1H), 3.94 (s, 3H), 3.91-3.73 (m, 3H), 3.72-3.69 (m, 1H), 3.45 (s, 2H), 3.25-3.21 (m, 2H), 2.93-2.91 (m, 2H), 2.75-2.72 (m, 2H), 2.66-2.64 (m, 2H), 2.29-2.26 (m, 1H), 2.07-2.02 (m, 2H).

This material, as a solution in MeOH, was treated with excess of 2M HCl in diethyl ether and evaporated to dryness to provide the dihydrochloride salt of the title compound.

### EXAMPLE 19

### 6-({[((2S)-4-{2-[3-fluoro-6-(methyloxy)-4-quinolinyl]ethyl}-2-morpholinyl)methyl]amino}methyl)-2H-pyrido[3,2-b][1,4]oxazin-3(4H)-one

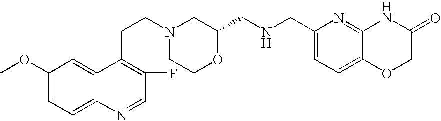

The title compound (84 mg, 52%) was prepared as a yellow solid according to Example 16, except substituting [((2S)-4-{2-[3-fluoro-6-(methyloxy)-4-quinolinyl]ethyl}-2-morpholinyl)methyl]amine (132 mg, 0.336 mmol) for 4-{2-[(2S)-2-(aminomethyl)morpholin-4-yl]ethyl}-6-methoxy-1,5-naphthyridine-3-carbonitrile and using (59 mg, 0.331 mmol) aldehyde: LC/MS (+ve ion electrospray) m/z 482 (M+H)⁺; ¹H NMR (CHCl₃, 400 MHz) δ 8.60 (s, 1H), 8.02 (d, J=9.22 Hz, 1H), 7.32 (d, J=6.55 Hz, 1H), 7.19 (m, 2H), 6.94 (d, J=8.06 Hz, 1H), 4.63 (s, 2H), 3.95 (s, 3H), 3.94-3.83 (m, 3H), 3.73-3.69 (m, 1H), 3.24-3.22 (m, 2H), 2.92-2.89 (m, 2H), 2.74-2.76 (m, 2H), 2.67-2.65 (m, 2H), 2.29-2.27 (m, 1H), 2.07-2.03 (m, 2H).

This material, as a solution in MeOH, was treated with excess of 2M HCl in diethyl ether and evaporated to dryness to provide the dihydrochloride salt of the title compound.

### EXAMPLE 20

### 8-fluoro-6-({[((2S)-4-{2-[3-fluoro-6-(methyloxy)-1,5-naphthyridin-4-yl]ethyl}-2-morpholinyl)methyl]amino}methyl)-2H-1,4-benzoxazin-3(4H)-one

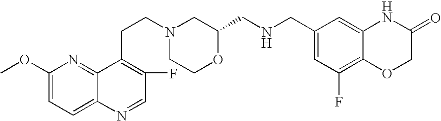

The title compound (148 mg, 55%) was prepared as a yellow solid according to Example 15, using free based amine (274 mg, 0.855 mmol) except substituting 8-fluoro-3-oxo-3,4-dihydro-2H-1,4-benzoxazine-6-carbaldehyde (105 mg, 0.538 mmol) for 3-oxo-3,4-dihydro-2H-pyrido[3,2-b][1,4]oxazine-6-carbaldehyde: LC/MS (+ve ion electrospray) m/z 500 (M+H)⁺; ¹H NMR (CHCl₃, 400 MHz) δ 8.61 (s, 1H), 8.18 (d, J=9.05 Hz, 1H), 7.06 (d, J=9.04 Hz, 1H), 6.78 (d, J=9.05 Hz, 1H), 6.63 (s, 1H), 4.65 (s, 2H), 4.06 (s, 3H), 3.90-3.87 (m, 1H), 3.74-3.61 (m, 4H), 3.39-3.35 (m, 2H), 2.89-2.87 (m, 2H), 2.78-2.75 (m, 2H), 2.71-2.58 (m, 2H), 2.31-2.28 (m, 1H), 2.06-2.03 (m, 1H).

This material, as a solution in MeOH, was treated with excess of 2M HCl in diethyl ether and evaporated to dryness to provide the dihydrochloride salt of the title compound.

### EXAMPLE 21

### 7-chloro-6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]oxazin-3(4H)-one

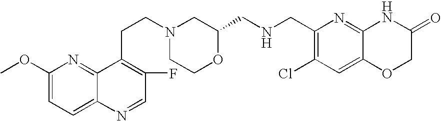

The title compound (148 mg, 55%) was prepared as a yellow solid according to Example 15, using free based amine (107 mg, 0.334 mmol) except substituting 7-chloro-3-oxo-3,4-dihydro-2H-pyrido[3,2-b][1,4]oxazine-6-carbaldehyde (71 mg, 0.334 mmol) for 3-oxo-3,4-dihydro-2H-pyrido[3,2-b][1,4]oxazine-6-carbaldehyde: LC/MS (+ve ion electrospray) m/z 517 (M+H)⁺; ¹H NMR (CHCl₃, 400 MHz) δ 8.60 (s, 1H), 8.18 (d, J=9.04 Hz, 1H), 7.20 (d, J=1.59 Hz, 1H), 7.06 (d, J=9.04 Hz, 1H), 4.59 (s, 2H), 4.06 (s, 3H), 3.99-3.87 (m, 3H), 3.81-3.78 (m, 1H), 3.68-3.64 (m, 1H), 3.40-3.37 (m, 2H), 2.97-2.93 (m, 2H), 2.79-2.75 (m, 4H), 2.31-2.28 (m, 1H), 2.07-2.05 (m, 1H).

This material, as a solution in MeOH, was treated with excess of 2M HCl in diethyl ether and evaporated to dryness to provide the dihydrochloride salt of the title compound.

### EXAMPLE 22

### [((2S)-4-{2-[3-fluoro-6-(methyloxy)-1,5-naphthyridin-4-yl]ethyl}-2-morpholinyl)methyl]([1,3]oxathiolo[5,4-c]pyridin-6-ylmethyl)amine

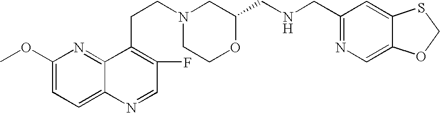

The title compound (139 mg, 96%) was prepared as a light yellow solid according to Example 15, using free based amine (98 mg, 0.306 mmol) except substituting [1,3]oxathiolo[5,4-c]pyridine-6-carbaldehyde (51 mg, 0.305 mmol) for 3-oxo-3,4-dihydro-2H-pyrido[3,2-b][1,4]oxazine-6-carbaldehyde: LC/MS (+ve ion electrospray) m/z 472 (M+H)⁺; ¹H NMR (CHCl₃, 400 MHz) δ 8.52 (s, 1H), 8.08 (d, J=9.04 Hz, 1H), 7.92 (s, 1H), 7.12 (s, 1H), 6.98 (d, J=9.04 Hz, 1H), 5.63 (s, 2H), 4.15 (br s, 1H), 3.98 (s, 3H), 3.80-3.44 (m, 4H), 3.31-3.28 (m, 2H), 2.82-2.77 (m, 2H), 2.67-2.57 (m, 4H), 2.21-2.16 (m, 1H), 1.95-1.93 (m, 1H).

This material, as a solution in MeOH, was treated with excess of 2M HCl in diethyl ether and evaporated to dryness to provide the dihydrochloride salt of the title compound.

### EXAMPLE 23

### 7-({[((2S)-4-{2-[3-fluoro-6-(methyloxy)-1,5-naphthyridin-4-yl]ethyl}-2-morpholinyl)methyl]amino}methyl)-2,3-dihydro-1,4-benzodioxin-5-carbonitrile

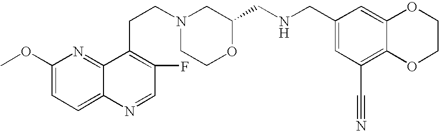

The title compound (68 mg, 35%) was prepared as a pale yellow solid according to Example 15, using free based amine (125 mg, 0.390 mmol) except substituting 7-formyl-2,3-dihydro-1,4-benzodioxin-5-carbonitrile (72 mg, 0.381 mmol) for 3-oxo-3,4-dihydro-2H-pyrido[3,2-b][1,4]oxazine-6-carbaldehyde: LC/MS (+ve ion electrospray) m/z 494 (M+H)⁺;

¹H NMR (CHCl₃, 400 MHz) δ 8.54 (s, 1H), 8.10 (d, J=9.04 Hz, 1H), 7.02-6.99 (m, 3H), 4.59 (br s, 1H), 4.31-4.22 (m, 4H), 4.00 (s, 3H), 3.84-3.81 (m, 1H), 3.64-3.60 (m, 3H), 3.34-3.30 (m, 2H), 2.83-2.81 (m, 2H), 2.70-2.66 (m, 2H), 2.60-2.57 (m, 2H), 2.24-2.20 (m, 1H), 2.01-1.95 (m, 2H).

This material, as a solution in MeOH, was treated with excess of 2M HCl in diethyl ether and evaporated to dryness to provide the dihydrochloride salt of the title compound.

### EXAMPLE 24

### 5-({[((2S)-4-{2-[3-fluoro-6-(methyloxy)-1,5-naphthyridin-4-yl]ethyl}-2-morpholinyl)methyl]amino}methyl)-2,3-dihydro-1-benzofuran-7-carbonitrile

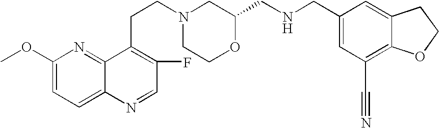

The title compound (66 mg, 36%) was prepared as a light yellow solid according to Example 15, using free based amine (122 mg, 0.381 mmol) except substituting 5-formyl-2,3-dihydro-1-benzofuran-7-carbonitrile (66 mg, 0.379 mmol) for 3-oxo-3,4-dihydro-2H-pyrido[3,2-b][1,4]oxazine-6-carbaldehyde: LC/MS (+ve ion electrospray) m/z 478 (M+H)⁺;

¹H NMR (CHCl₃, 400 MHz) δ 8.54 (s, 1H), 8.11 (d, J=9.04 Hz, 1H), 7.32 (s, 1H), 7.18 (s, 1H), 7.00 (d, J=9.05 Hz, 1H), 4.67-4.63 (m, 2H), 3.99 (s, 3H), 3.83 (m, 1H), 3.65-3.60 (m, 3H), 3.32 (m, 2H), 3.20-3.16 (m, 2H), 2.85-2.80 (m, 2H), 2.70-2.65 (m, 2H), 2.60-2.55 (m, 2H), 2.23-2.19 (m, 1H), 2.00-1.95 (m, 2H).

This material, as a solution in MeOH, was treated with excess of 2M HCl in diethyl ether and evaporated to dryness to provide the dihydrochloride salt of the title compound.

### EXAMPLE 25

### 6-({[((2S)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one (D₁ and D₂)

### (a) 1,1-dimethylethyl[((2S)-4-{2-[3-fluoro-6-(methyloxy)-1,5-naphthyridin-4-yl]-2-hydroxyethyl}-2-morpholinyl)methyl]carbamate

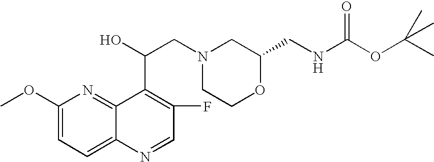

7-fluoro-2-(methyloxy)-8-(2-oxiranyl)-1,5-naphthyridine (175 mg, 0.795 mmol) and 1,1-dimethylethyl[(2R)-2-morpholinylmethyl]carbamate (182 mg, 0.842 mmol) were mixed in EtOH (2 mL) and heated at 70° C. over 12 h. The solution was concentrated and the residue purified via column chromatography (silica, 10% MeOH in DCM with 1% NH₄OH) yielding the title compound (340 mg, 98%) as an oil; LC/MS (+ve ion electrospray) m/z 437 (M+H)⁺.

### (b) 2-[(2S)-2-(aminomethyl)-4-morpholinyl]-1-[3-fluoro-6-(methyloxy)-1,5-naphthyridin-4-yl]ethanol

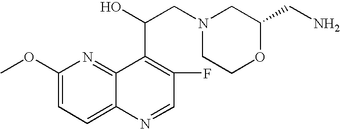

To a solution of 1,1-dimethylethyl[(4-{2-[3-fluoro-6-(methyloxy)-1,5-naphthyridin-4-yl]-2-hydroxyethyl}-2-morpholinyl)methyl]carbamate (340 mg, 0.779 mmol) in DCM (10 mL) and MeOH (1 mL) was added dropwise a solution of HCl in dioxane (2.7 mL, 4.8 mmol, 4 MHCl in dioxane). After 12 h, the reaction was concentrated to afford the HCl salt of the title compound (310 mg, 97%) as a white solid, which was used without further purification: LC/MS (+ve ion electrospray) m/z 337 (M+H)⁺.

### (c) 6-({[((2S)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one

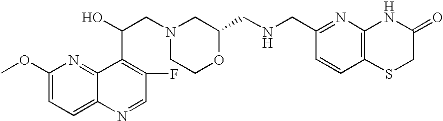

The title compound (255 mg, 64%) was prepared as a mixture of diastereomers to give a light yellow solid according to Example 10, except substituting 2-[(2S)-2-(aminomethyl)-4-morpholinyl]-1-[3-fluoro-6-(methyloxy)-1,5-naphthyridin-4-yl]ethanol (310 mg, 0.758 mmol) for (1R)-2-[(2S)-2-(aminomethyl)-4-morpholinyl]-1-[6-(methyloxy)-1,5-naphthyridin-4-yl]ethanol and reacting that with 3-oxo-3,4-dihydro-2H-pyrido[3,2-b][1,4]thiazine-6-carbaldehyde (166 mg, 0.855 mmol).

The mixture of diastereomers underwent purification by chiral HPLC (chiralpak AD). The faster eluting diastereomer was assigned D1: LC/MS (+ve ion electrospray) m/z 515 (M+H)⁺; ¹H NMR (CHCl₃, 400 MHz) δ 9.22 (br. s, 1H), 8.65 (s, 1H), 8.27 (d, J=9.15 Hz, 1H), 7.55 (d, J=7.81 Hz, 1H), 7.12 (d, J=9.14 Hz, 1H), 6.96 (d, J=7.83 Hz, 1H), 5.63-5.58 (m, 1H), 4.05 (s, 3H), 3.87-3.81 (m, 3H), 3.71-3.65 (m, 2H), 3.45 (s, 2H), 3.12-3.05 (m, 1H), 2.92-2.85 (m, 1H), 2.75-2.60 (m, 4H) 2.37-2.32 (m, 1H), 2.09-2.04 (m, 1H). The slower eluting diastereomer was assigned D2: LC/MS (+ve ion electrospray) m/z 515 (M+H)⁺: ¹H NMR (CHCl₃, 400 MHz) δ 9.15 (br. s, 1H), 8.66 (s, 1H), 8.27 (d, J=9.14 Hz, 1H), 7.57 (d, J=7.81 Hz, 1H), 7.12 (d, J=9.14 Hz, 1H), 6.98 (d, J=7.83 Hz, 1H), 5.67-5.58 (m, 1H), 4.05 (s, 3H), 3.85-3.82 (m, 3H), 3.77-3.73 (m, 1H), 3.65-3.59 (m, 1H), 3.47 (s, 2H), 3.09-3.01 (m, 1H), 2.96-2.89 (m, 1H), 2.76-2.66 (m, 4H) 2.36-2.29 (m, 1H), 2.15-2.09 (m, 1H).

These solids, as a solution in MeOH, were treated with excess of 2M HCl in diethyl ether and evaporated to dryness to provide the dihydrochloride salt of the title compound.

### EXAMPLE 26

### 6-({[((2R)-4-{2-[3-fluoro-6-(methyloxy)-1,5-naphthyridin-4-yl]-2-hydroxyethyl}-2-morpholinyl)methyl]amino}methyl)-2H-pyrido[3,2-b][14]thiazin-3(4H)-one (D₁ and D₂)

### (a) 1,1-dimethylethyl[((2R)-4-{2-[3-fluoro-6-(methyloxy)-1,5-naphthyridin-4-yl]-2-hydroxyethyl}-2-morpholinyl)methyl]carbamate

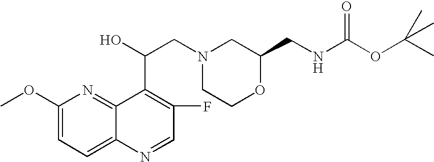

The title compound (393 mg, 100%) was prepared as a colorless oil according to Example 25a, except substituting 1,1-dimethylethyl[(2S)-2-morpholinylmethyl]carbamate (227 mg, 1.05 mmol) for 1,1-dimethylethyl[(2R)-2-morpholinylmethyl]carbamate and using 7-fluoro-2-(methyloxy)-8-(2-oxiranyl)-1,5-naphthyridine (198 mg, 0.90 mmol): LC/MS (+ve ion electrospray) m/z 437 (M+H)⁺.

### (b) 2-[(2R)-2-(aminomethyl)-4-morpholinyl]-1-[3-fluoro-6-(methyloxy)-1,5-naphthyridin-4-yl]ethanol

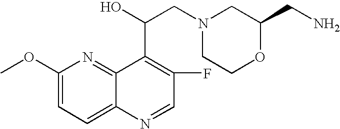

The title compound (368 mg, 100%) was prepared as a white solid according to Example 25b, which was used without further purification: LC/MS (+ve ion electrospray) m/z 337 (M+H)⁺.

### (c) 6-({[((2R)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4″-one

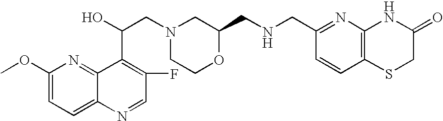

The title compound (315 mg, 100%) was prepared as a white solid mixture of diastereomers according to Example 25c, except substituting 2-[(2R)-2-(aminomethyl)-4-morpholinyl]-1-[3-fluoro-6-(methyloxy)-1,5-naphthyridin-4-yl]ethanol (417 mg, 1.24 mmol) 2-[(2S)-2-(aminomethyl)-4-morpholinyl]-1-[3-fluoro-6-(methyloxy)-1,5-naphthyridin-4-yl]ethanol and reacting that with 3-oxo-3,4-dihydro-2H-pyrido[3,2-b][1,4]thiazine-6-carbaldehyde (238 mg, 1.23 mmol).

The mixture of diastereomers underwent purification by chiral HPLC (chiralpak AD). The faster eluting diastereomer was assigned D1: LC/MS (+ve ion electrospray) m/z 515 (M+H)⁺; ¹H NMR (CHCl₃, 400 MHz) δ 8.66 (s, 1H), 8.26 (d, J=9.14 Hz, 1H), 7.58 (d, J=7.81 Hz, 1H), 7.12 (d, J=9.14 Hz, 1H), 7.00 (d, J=7.83 Hz, 1H), 5.66-5.60 (m, 1H), 4.05 (s, 3H), 3.89-3.78 (m, 4H), 3.66-3.60 (m, 1H), 3.47 (s, 2H), 3.09-3.02 (m, 1H), 2.97-2.93 (m, 1H), 2.76-2.71 (m, 4H) 2.35-2.30 (m, 1H), 2.15-2.10 (m, 1H). The slower eluting diastereomer was assigned D2: LC/MS (+ve ion electrospray) m/z 515 (M+H)⁺: ¹H NMR (CHCl₃, 400 MHz) δ 8.66 (s, 1H), 8.27 (d, J=9.15 Hz, 1H), 7.63 (d, J=7.82 Hz, 1H), 7.14 (d, J=9.15 Hz, 1H), 7.00 (d, J=7.86 Hz, 1H), 5.67-5.62 (m, 1H), 4.31-4.04 (s, 6H), 4.01-3.92 (m, 1H), 3.81-2.75 (m, 1H), 3.59-3.45 (m, 3H), 3.21-2.72 (m, 6H), 2.53-2.47 (m, 1H), 2.26-2.17 (m, 1H).

These solids, as a solution in MeOH, were treated with excess of 2M HCl in diethyl ether and evaporated to dryness to provide the dihydrochloride salt of the title compound.

### EXAMPLE 27

### Preparation of 6-[(8-{2-[3-fluoro-6-(methyloxy)-1,5-naphthyridin-4-yl]ethyl}-4-oxooctahydro-2H-pyrazino[1,2-a]pyrazin-2-yl)methyl]-2H-pyrido[3,2-b][1,4]thiazin-3(4H)-one

### a) 1,1-dimethylethyl[(1-(chloroacetyl)-4-{2-[3-fluoro-6-(methyloxy)-1,5-naphthyridin-4-yl]ethyl}-2-piperazinyl)methyl]carbamate

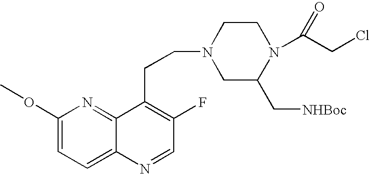

To a solution of 1,1-dimethylethyl[(4-{2-[3-fluoro-6-(methyloxy)-1,5-naphthyridin-4-yl]ethyl}-2-piperazinyl)methyl]carbamate (400 mg, 0.955 mmol) [prepared from Example 13] in THF (8 mL) at 0° C. was added Et₃N (268 μL, 1.91 mmol) followed by chloroacetyl chloride (76 μL, 0.239 mmol). After 1 h, the reaction was partitioned between a saturated solution of Na₂CO₃ and DCM. The layers were separated and the aqueous phase was back extracted several times with DCM. The combined organic fractions were dried (Na₂SO₄), concentrated and purified via column chromatography (silica, 1% MeOH in DCM (1% NH₄OH)) affording the title compound (400 mg, 85%) as a yellow foam: LCMS (ES) m/e 496 (M+H)⁺.

### b) 1,1-dimethylethyl 8-{2-[3-fluoro-6-(methyloxy)-1,5-naphthyridin-4-yl]ethyl}-4-oxooctahydro-2H-pyrazino[1,2-a]pyrazine-2-carboxylate

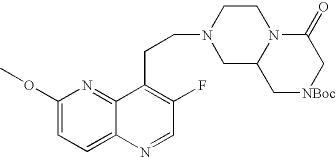

To a solution of 1,1-dimethylethyl[(1-(chloroacetyl)-4-{2-[3-fluoro-6-(methyloxy)-1,5-naphthyridin-4-yl]ethyl}-2-piperazinyl)methyl]carbamate (400 mg, 0.808 mmol) in THF-DMF (12 mL, 12:1) at 0° C. was added NaH (97 mg, 2.42 mmol, 60% in mineral oil) portion-wise. After 4 h at 25° C., the solution was quenched with H₂O and the aqueous phase was washed several times with DCM. The combined organic fractions were dried (Na₂SO₄), concentrated and the residue was used directly in the following procedure: LCMS (ES) m/e 460 (M+H)⁺.

### c) 8-{2-[3-fluoro-6-(methyloxy)-1,5-naphthyridin-4-yl]ethyl}octahydro-4H-pyrazino[1,2-a]pyrazin-4-one

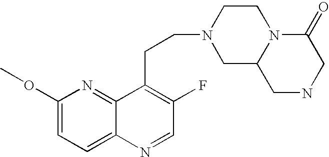

To a solution of the Boc-amine (372 mg, 0.808 mmol) in MeOH (8 mL) at 25° C. was added dropwise a solution of HCl in dioxane (1.4 mL, 5.66 mmol, 4M HCl in dioxane). After 12 h, the solution was concentrated and the residue neutralized by addition of excess Et₃N to the salt in DCM. The solution was concentrated and purified through a pad of silica (3% MeOH in DCM (1% NH₄OH) affording the title compound (291 mg, quant.) as a clear oil: LCMS (ES) m/e 360 (M+H)⁺.

### d) 6-[(8-{2-[3-fluoro-6-(methyloxy)-1,5-naphthyridin-4-yl]ethyl}-4-oxooctahydro-2H-pyrazino[1,2-a]pyrazin-2-yl)methyl]-2H-pyrido[3,2-b][1,4]thiazin-3(4H)-one

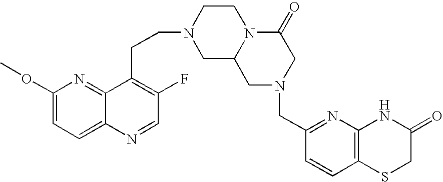

To a solution of 8-{2-[3-fluoro-6-(methyloxy)-1,5-naphthyridin-4-yl]ethyl}octahydro-4H-pyrazino[1,2-a]pyrazin-4-one (130 mg, 0.361 mmol) and 3-oxo-3,4-dihydro-2H-pyrido[3,2-b][1,4]thiazine-6-carbaldehyde (70 mg, 0.361 mmol) in DCE (4 mL) was added Na(OAc)₃BH (115 mg, 0.542 mmol). After 2 h, the solution was partitioned between a saturated solution of Na₂CO₃ and DCM. The aqueous phase was back-extracted several times with DCM and the combined organic fractions were dried (Na₂SO₄), concentrated and purified by column chromatography (silica, 0.5-2% MeOH in DCM (1% NH₄OH)) yielding the title compound (100 mg, 52%) as a light yellow foam: LCMS (ES) m/e 538 (M+H)⁺; ¹H NMR (CD₃OD, 400 MHz) δ 8.63 (s, 1H), 8.18 (d, J=9.0 Hz, 1H), 7.69 (d, J=7.7 Hz, 1H), 7.15 (d, J=9.0 Hz, 1H), 7.08 (d, J=7.8 Hz, 1H), 4.24-4.65 (m, 2H), 4.06 (s, 3H), 3.57-3.66 (m, 2H), 3.52 (s, 2H), 3.37-3.51 (m, 2H), 3.30-3.33 (m, 1H), 3.07-3.15 (m, 2H), 3.00-3.05 (m, 2H), 2.72-2.82 (m, 3H), 2.33-2.40 (m, 1H), 2.10-2.16 (m, 1H), 1.99-2.05 (m, 1H).

This material, as a solution in MeOH, was treated with an excess of 4M HCl in dioxane and evaporated to dryness to provide the dihydrochloride salt of the title compound.

### EXAMPLE 28

### Preparation of 6-[(8-{2-[3-fluoro-6-(methyloxy)-1,5-naphthyridin-4-yl]ethyl}-3-oxooctahydro-2H-pyrazino[1,2-a]pyrazin-2-yl)methyl]-2H-pyrido[3,2-b][1,4]thiazin-3(4H)-one

### a) 9H-fluoren-9-ylmethyl 2-[({[(1,1-dimethylethyl)oxy]carbonyl}amino)methyl]-4-{2-[3-fluoro-6-(methyloxy)-1,5-naphthyridin-4-yl]ethyl}-1-piperazinecarboxylate

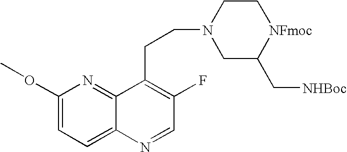

To a solution of 1,1-dimethylethyl[(4-{2-[3-fluoro-6-(methyloxy)-1,5-naphthyridin-4-yl]ethyl}-2-piperazinyl)methyl]carbamate (460 mg, 1.10 mmol) [prepared from Example 13] in DCM (11 mL) at 0° C. was added Et₃N (306 μL, 2.2 mmol) followed by 9-fluorenylmethyl chloroformate (312 mg, 1.2 mmol) portion-wise. After 1 h, the reaction was partitioned between H₂O and DCM. The layers were separated and the aqueous phase was back extracted several times with DCM. The combined organic fractions were dried (Na₂SO₄), concentrated and purified via column chromatography (silica, 1% MeOH in DCM) affording the title compound (580 mg, 82%) as a yellow foam: LCMS (ES) m/e 642 (M+H)⁺.

b) 9H-fluoren-9-ylmethyl 2-(aminomethyl)-4-{2-[3-fluoro-6-(methyloxy)-1,5-naphthyridin-4-yl]ethyl}-1-piperazinecarboxylate

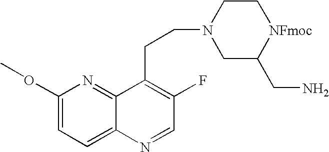

To a solution of the Boc-amine (580 mg, 0.905 mmol) in MeOH (9 mL) at 25° C. was added dropwise a solution of HCl in dioxane (1.5 mL, 6.33 mmol, 4M HCl in dioxane). After 12 h, the solution was concentrated and the residue neutralized by addition of excess Et₃N to the salt in DCM. The solution was concentrated and purified through a pad of silica (5% MeOH in DCM (1% NH₄OH) affording the title compound (480 mg, quant.) as a clear oil: LCMS (ES) m/e 542 (M+H)⁺.

### c) 9H-fluoren-9-ylmethyl 4-{2-[3-fluoro-6-(methyloxy)-1,5-naphthyridin-4-yl]ethyl}-2-({[(3-oxo-3,4-dihydro-2H-pyrido[3,2-b][1,4]thiazin-6-yl)methyl]amino}methyl)-1-piperazinecarboxylate

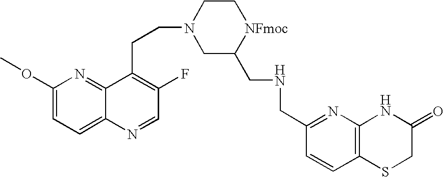

To a solution of 9H-fluoren-9-ylmethyl 2-(aminomethyl)-4-{2-[3-fluoro-6-(methyloxy)-1,5-naphthyridin-4-yl]ethyl}-1-piperazinecarboxylate (425 mg, 0.786 mmol) and 3-oxo-3,4-dihydro-2H-pyrido[3,2-b][1,4]thiazine-6-carbaldehyde (153 mg, 0.786 mmol) in DCM-EtOH (8 mL, 15:1) was added Na(OAc)₃BH (250 mg, 1.20 mmol). After 2 h, the solution was partitioned between a saturated solution of Na₂CO₃ and DCM. The aqueous phase was back-extracted several times with DCM and the combined organic fractions were dried (Na₂SO₄), concentrated and purified by column chromatography (silica, 1% MeOH in DCM (10% NH₄OH)) yielding the title compound (490 mg, 75%) as a light yellow foam: LCMS (ES) m/e 720 (M+H)⁺.

### d) 9H-fluoren-9-ylmethyl 2-({(chloroacetyl)[(3-oxo-3,4-dihydro-2H-pyrido[3,2-b][1,4]thiazin-6-yl)methyl]amino}methyl)-4-{2-[3-fluoro-6-(methyloxy)-1,5-naphthyridin-4-yl]ethyl}-1-piperazinecarboxylate

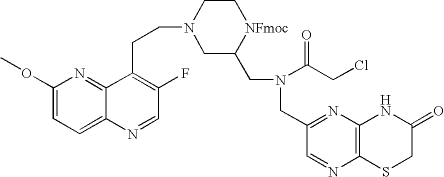

To a solution of the amine (490 mg, 0.682 mmol) in THF (7 mL) at 0° C. was added Et₃N (190 μL, 1.36 mmol) followed by dropwise addition of chloroacetyl chloride (65 μL, 0.818 mmol). After 2 h, the solution was partitioned between H₂O-DCM. The aqueous phase was back-extracted several times with DCM and the combined organic fractions were dried (Na₂SO₄), concentrated and purified by column chromatography (silica, 1% MeOH in DCM) yielding the title compound (500 mg, 92%) as a yellow foam: LCMS (ES) m/e 796 (M+H)⁺.

### e) 6-[(8-{2-[3-fluoro-6-(methyloxy)-1,5-naphthyridin-4-yl]ethyl}-3-oxooctahydro-2H-pyrazino[1,2-a]pyrazin-2-yl)methyl]-2H-pyrido[3,2-b][1,4]thiazin-3(4H)-one

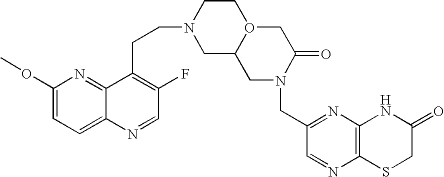

To a solution of the Fmoc-amine (90 mg, 0.113 mmol) in THF (1 mL) at 0° C. was added dropwise a solution of TBAF (136 μL, 0.136 mmol, 1 M in THF). After warming to 25° C. over 12 h, additional TBAF (57 μL, 57 μmol) was added at this temperature and the solution stirred for 6 h further. The resulting mixture was partitioned between H₂O-DCM and the aqueous phase was back-extracted several times with DCM. The combined organic fractions were dried (Na₂SO₄), concentrated and purified by column chromatography (silica, 1% MeOH in DCM (1% NH₄OH)) yielding the title compound (15 mg, 25%) as a white foam: LCMS (ES) m/e 538 (M+H)⁺; ¹H NMR (CD₃OD, 400 MHz) δ 8.65 (s, 1H), 8.21 (d, J=9.0 Hz, 1H), 7.71 (d, J=7.8 Hz, 1H), 7.19 (d, J=9.0 Hz, 1H), 6.97 (d, J=7.6 Hz, 1H), 4.59 (AB quartet, 2H), 4.10 (s, 3H), 3.53 (s, 2H), 3.47-3.53 (m, 2H), 3.32-3.36 (m, 3H (obscured by CD₃OD signal)), 3.11-3.17 (m, 2H), 2.92-3.03 (m, 2H), 2.82-2.90 (m, 2H), 2.61-2.65 (m, 1H), 2.39-2.45 (m, 2H), 2.02-2.11 (m, 1H).

This material, as a solution in MeOH, was treated with an excess of 4M HCl in dioxane and evaporated to dryness to provide the dihydrochloride salt of the title compound.

### EXAMPLE 29

### Preparation of 6-({[(4-{2-[6-(methyloxy)-1,5-naphthyridin-4-yl]ethyl}-2-piperazinyl)methyl]amino}methyl)-2H-pyrido[3,2-b][1,4]thiazin-3(4H)-one

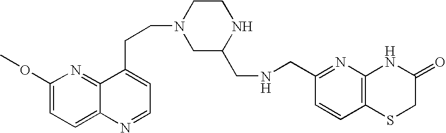

The title compound (105 mg, 35%) was prepared as a yellow foam according to Example 13, except substituting 8-ethenyl-2-(methyloxy)-1,5-naphthyridine (365 mg, 1.96 mmol) for 8-ethenyl-7-fluoro-2-(methyloxy)-1,5-naphthyridine: LCMS (ES) m/e 480 (M+H)⁺; ¹H NMR (CD₃OD, 400 MHz) δ 8.62 (d, J=4.5 Hz, 1H), 8.19 (d, J=9.0 Hz, 1H), 7.69 (d, J=7.8 Hz, 1H), 7.60 (d, J=4.5 Hz, 1H), 7.23 (d, J=9.0 Hz, 1H), 7.03 (d, J=7.8 Hz, 1H), 4.10 (s, 3H), 3.80 (s, 2H), 3.53 (s, 2H), 3.43-3.48 (m, 2H), 3.00-3.08 (m, 3H), 2.83-2.92 (m, 4H), 2.61 (d, J=6.3 Hz, 2H), 2.21-2.29 (m, 1H), 1.95-1.99 (m, 1H).

This material, as a solution in MeOH, was treated with an excess of 4M HCl in dioxane and evaporated to dryness to provide the dihydrochloride salt of the title compound.

### EXAMPLE 30

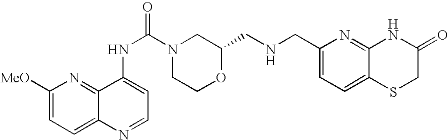

### Preparation of (2S)—N-[6-(methyloxy)-1,5-naphthyridin-4-yl]-2-({[(3-oxo-3,4-dihydro-2H-pyrido[3,2-b][1,4]thiazin-6-yl)methyl]amino}methyl)-4-morpholinecarboxamide

### a) 1,1-dimethylethyl {[(2S)-4-({[6-(methyloxy)-1,5-naphthyridin-4-yl]amino}carbonyl)-2-morpholinyl]methyl}carbamate

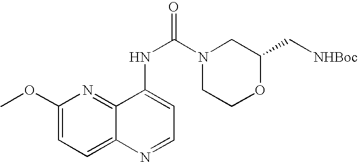

To a solution of 6-(methyloxy)-1,5-naphthyridin-4-amine (0.35 g, 2.0 mmole) in CHCl₃ (10 mL) at RT was added DMAP (0.24 g, 2.0 mmole) and carbonyldiimidazole (0.42 g, 2.6 mmole). After 5 h, the reaction contents were concentrated in vacuo to a solid. The residue was dissolved in DMF (5 mL) and 1,1-dimethylethyl[(2R)-2-morpholinylmethyl]carbamate (0.47 g, 2.2 mmole) was added. After 3 hours at 100° C., the reaction solution was concentrated and purified on silica (CHCl₃/MeOH, 9:1) to give the title compound (68%, 0.57 g) as an off-white solid: LC-MS (EI) m/z 418 (M+H)⁺.

### b)(2S)—N-[6-(methyloxy)-1,5-naphthyridin-4-yl]-2-({[(3-oxo-3,4-dihydro-2H-pyrido[3,2-b][1,4]thiazin-6-yl)methyl]amino}methyl)-4-morpholinecarboxamide

To a solution of 1,1-dimethylethyl {[(2S)-4-({[6-(methyloxy)-1,5-naphthyridin-4-yl]amino}carbonyl)-2-morpholinyl]methyl}carbamate (0.40 g, 0.95 mmole) in MeOH (5 mL) was added HCl (10 mL, 4M in diaxane). After 5 hrs at RT, the suspension was concentrated under vacuum. To the remaining residue in EtOH (10 mL) and CHCl₃ (40 mL) was added (i-Pr)₂NEt (0.66 mL), and 3-oxo-3,4-dihydro-2H-pyrido[3,2-b][1,4]thiazine-6-carbaldehyde (0.20 g, 1.05 mmole). After stirring for 12 hr, NaBH₄ (40 mg, 1.05 mmole) was added to the reaction solution and stirring was allowed to continue for 2 additional hours. Silica gel was added to the reaction solution and the contents concentrated under vacuum. The solid contents were poured onto a silica gel column and the product eluted with [MeOH (5% NH₄OH)/CHCl₃, 1:9] to give the title compound (70%) as an off-white solid: LC-MS (EI) m/z 496 (M+H)⁺; ¹H NMR (CD₃OD, 400 Hz) δ 8.55 (d, J=5.5 Hz, 1H), 8.25 (d, J=5.5 Hz, 1H), 8.13 (d, J=9.0 Hz, 1H), 7.80 (d, J=7.8 Hz, 1H), 7.23 (d, J=9.0 Hz, 1H), 7.14 (d, J=7.7 Hz, 1H), 4.39 (s, 2H), 4.18 (m, 2H), 4.09 (s, 3H), 3.95 (m, 1H), 3.66-3.76 (m, 2H), 3.51 (s, 2H), 3.26-3.31 (m, 2H), 2.94 (m, 2H).

| Example. | Structure | Formula |
|---|---|---|
| 1 | 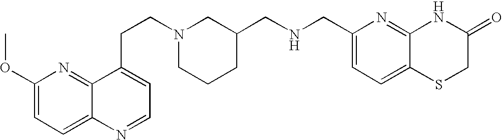 | 6-({[(1-{2-[6-(methyloxy)-1,5 naphthyridin-4-yl]ethyl}-3- piperidinyl)methyl]amino}methyl)-2H- pyrido[3,2-b][1,4]thiazin-3(4H)-one |
| 2 | 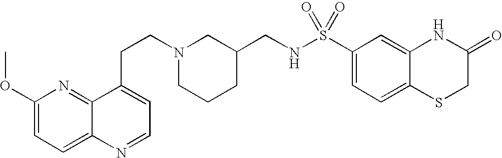 | N-[(1-{2-[6-(methyloxy)-1,5-naphthyridin- 4-yl]ethyl}-3-piperidinyl)methyl]-3-oxo- 3,4-dihydro-2H-1,4-benzothiazine-6- sulfonamide |
| 3 | 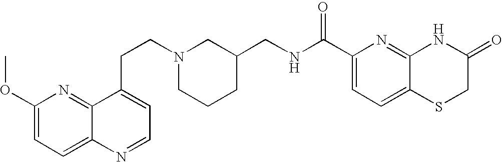 | N-[(1-{2-[6-(methyloxy)-1,5-naphthyridin- 4-yl]ethyl}-3-piperidinyl)methyl]-3-oxo- 3,4-dihydro-2H-pyrido[3,2- b][1,4]thiazine-6-carboxamide |
| 4 | 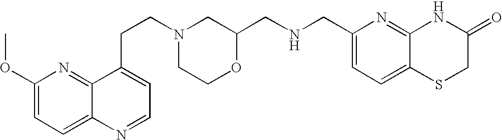 | 6-({[(4-{2-[6-(methyloxy)-1,5- naphthyridin-4-yl]ethyl}-2- morpholinyl)methyl]amino}methyl)-2H- pyrido[3,2-b][1,4]thiazin-3(4H)-one |
| 5 | 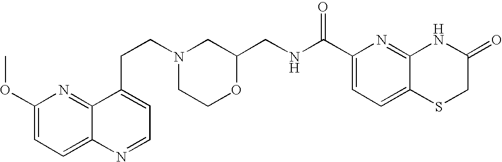 | N-[(4-{2-[6-(methyloxy)-1,5-naphthyridin- 4-yl]ethyl}-2-morpholinyl)methyl]-3-oxo- 3,4-dihydro-2H-pyrido[3,2- b][1,4]thiazine-6-carboxamide |
| 6 | 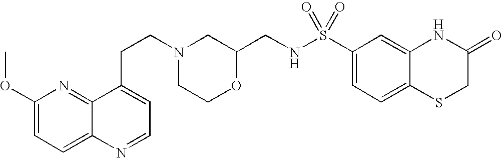 | N-[(4-{2-[6-(methyloxy)-1,5-naphthyridin- 4-yl]ethyl}-2-morpholinyl)methyl]-3-oxo- 3,4-dihydro-2H-1,4-benzothiazine-6- sulfonamide |
| 7 | 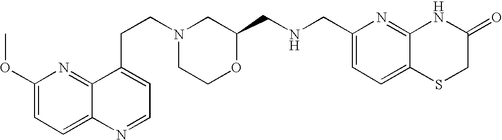 | 6-({[((2R)-4-{2-[6-(methyloxy)-1,5- naphthyridin-4-yl]ethyl}-2- morpholinyl)methyl]amino}methyl)-2H- pyrido[3,2-b][1,4]thiazin-3(4H)-one |
| 8 | 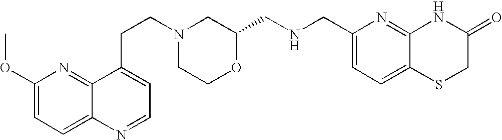 | 6-({[((2S)-4-{2-[6-(methyloxy)-1,5- naphthyridin-4-yl]ethyl}-2- morpholinyl)methyl]amino}methyl)-2H- pyrido[3,2-b][1,4]thiazin-3(4H)-one |
| 9 | 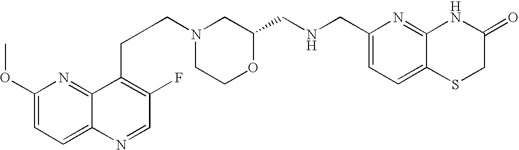 | 6-({[((2S)-4-{2-[3-fluoro-6-(methyloxy)- 1,5-naphthyridin-4-yl]ethyl}-2- morpholinyl)methyl]amino}methyl)-2H- pyrido[3,2-b][1,4]thiazin-3(4H)-one |
| 10 | 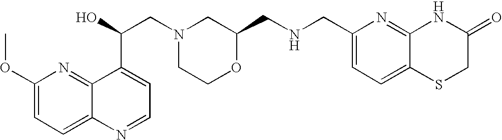 | 6-({[((2R)-4-{(2R)-2-hydroxy-2-[6- (methyloxy)-1,5-naphthyridin-4-yl]ethyl}- 2-morpholinyl)methyl]amino}methyl)-2H- pyrido[3,2-b][1,4]thiazin-3(4H)-one |
| 11 | 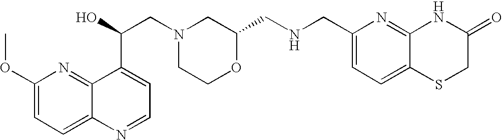 | 6-({[((2S)-4-{(2R)-2-hydroxy-2-[6- (methyloxy)-1,5-naphthyridin-4-yl]ethyl}- 2-morpholinyl)methyl]amino}methyl)-2H- pyrido[3,2-b][1,4]thiazin-3(4H)-one |
| 12 | 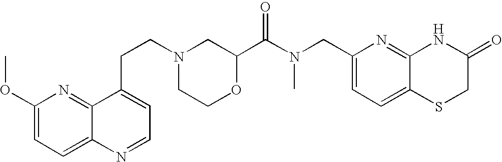 | N-methyl-4-{2-[6-(methyloxy)-1,5- naphthyridin-4-yl]ethyl}-N-[(3-oxo-3,4- dihydro-2H-pyrido[3,2-b][1,4]thiazin-6- yl)methyl]-2-morpholinecarboxamide |
| 13 | 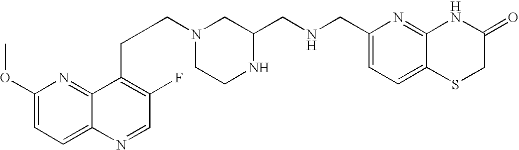 | 6-({[(4-{2-[3-fluoro-6-(methyloxy)-1,5- naphthyridin-4-yl]ethyl}-2- piperazinyl)methyl]amino}methyl)-2H- pyrido[3,2-b][1,4]thiazin-3(4H)-one |
| 14 | 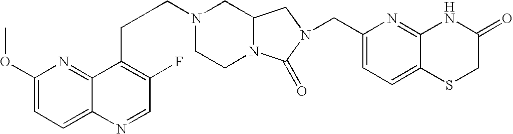 | 6-{[7-{2-[3-fluoro-6-(methyloxy)-1,5- naphthyridin-4-yl]ethyl}-3- oxohexahydroimidazo[1,5-a]pyrazin- 2(3H)-yl]methyl}-2H-pyrido[3,2- b][1,4]thiazin-3(4H)-one |
| 15 | 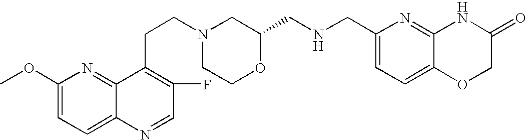 | 6-({[((2S)-4-{2-[3-fluoro-6-(methyloxy)- 1,5-naphthyridin-4-yl]ethyl}-2- morpholinyl)methyl]amino}methyl)-2H- pyrido[3,2-b][1,4]oxazin-3(4H)-one |
| 16 | 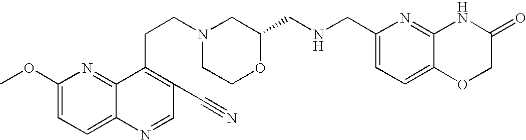 | 6-(methyloxy)-4-{2-[(2S)-2-({[(3-oxo-3,4- dihydro-2H-pyrido[3,2-b][1,4]oxazin-6- yl)methyl]amino}methyl)-4- morpholinyl]ethyl}-1,5-naphthyridine-3- carbonitrile |
| 17 | 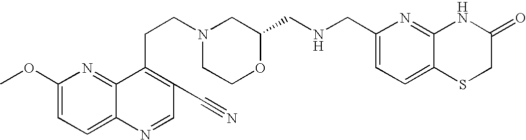 | 6-(methyloxy)-4-{2-[(2S)-2-({[(3-oxo-3,4- dihydro-2H-pyrido[3,2-b][1,4]thiazin-6- yl)methyl]amino}methyl)-4- morpholinyl]ethyl}-1,5-naphthyridine-3- carbonitrile |
| 18 | 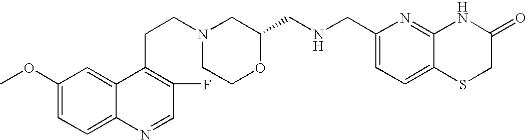 | 6-({[((2S)-4-{2-[3-fluoro-6-(methyloxy)-4- quinolinyl]ethyl}-2- morpholinyl)methyl]amino}methyl)-2H- pyrido[3,2-b][1,4]thiazin-3(4H)-one |
| 19 | 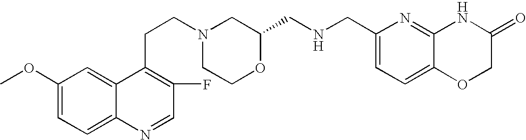 | 6-({[((2S)-4-{2-[3-fluoro-6-(methyloxy)-4- quinolinyl]ethyl}-2- morpholinyl)methyl]amino}methyl)-2H- pyrido[3,2-b][1,4]oxazin-3(4H)-one |
| 20 | 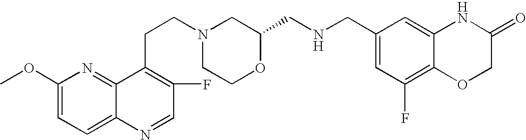 | 8-fluoro-6-({[((2S)-4-{2-[3-fluoro-6- (methyloxy)-1,5-naphthyridin-4-yl]ethyl}- 2-morpholinyl)methyl]amino}methyl)-2H- 1,4-benzoxazin-3(4H)-one |
| 21 | 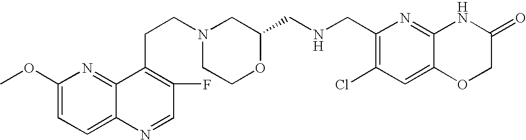 | 7-chloro-6-({[((2S)-4-{2-[3-fluoro-6- (methyloxy)-1,5-naphthyridin-4-yl]ethyl}- 2-morpholinyl)methyl]amino}methyl)-2H- pyrido[3,2-b][1,4]oxazin-3(4H)-one |
| 22 | 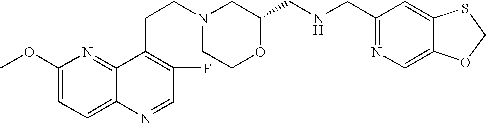 | [((2S)-4-{2-[3-fluoro-6-(methyloxy)-1,5- naphthyridin-4-yl]ethyl}-2- morpholinyl)methyl]([1,3]oxathiolo[5,4- c]pyridin-6-ylmethyl)amine |
| 23 | 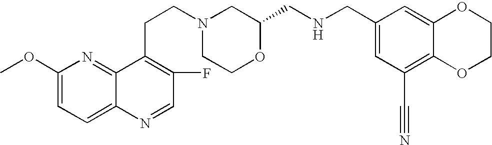 | 7-({[((2S)-4-{2-[3-fluoro-6-(methyloxy)- 1,5-naphthyridin-4-yl]ethyl}-2- morpholinyl)methyl]amino}methyl)-2,3- dihydro-1,4-benzodioxin-5-carbonitrile |
| 24 | 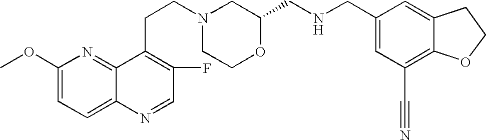 | 5-({[((2S)-4-{2-[3-fluoro-6-(methyloxy)- 1,5-naphthyridin-4-yl]ethyl}-2- morpholinyl)methyl]amino}methyl)-2,3- dihydro-1-benzofuran-7-carbonitrile |
| 25 D₁ | 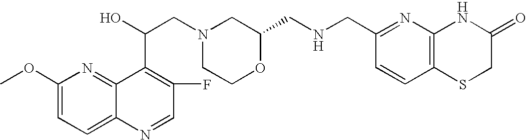 | 6-({[((2S)-4-{2-[3-fluoro-6-(methyloxy)- 1,5-naphthyridin-4-yl]-2-hydroxyethyl}-2- morpholinyl)methyl]amino}methyl)-2H- pyrido[3,2-b][1,4]thiazin-3(4H)-one |
| 25 D₂ | 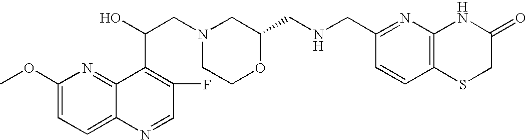 | 6-({[((2S)-4-{2-[3-fluoro-6-(methyloxy)- 1,5-naphthyridin-4-yl]-2-hydroxyethyl}-2- morpholinyl)methyl]amino}methyl)-2H- pyrido[3,2-b][1,4]thiazin-3(4H)-one |
| 26 D₁ | 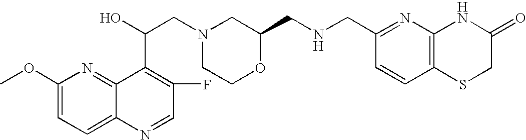 | 6-({[((2R)-4-{2-[3-fluoro-6-(methyloxy)- 1,5-naphthyridin-4-yl]-2-hydroxyethyl}-2- morpholinyl)methyl]amino}methyl)-2H- pyrido[3,2-b][1,4]thiazin-3(4H)-one |
| 26 D₂ | 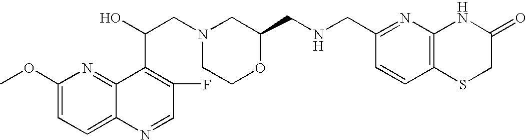 | 6-({[((2R)-4-{2-[3-fluoro-6-(methyloxy)- 1,5-naphthyridin-4-yl]-2-hydroxyethyl}-2- morpholinyl)methyl]amino}methyl)-2H- pyrido[3,2-b][1,4]thiazin-3(4H)-one |
| 27 | 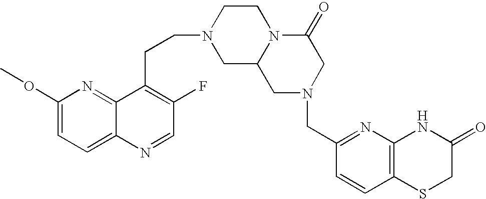 | 6-[(8-{2-[3-fluoro-6-(methyloxy)-1,5- naphthyridin-4-yl]ethyl}-4-oxooctahydro- 2H-pyrazino[1,2-a]pyrazin-2-yl)methyl]- 2H-pyrido[3,2-b][1,4]thiazin-3(4H)-one |
| 28 | 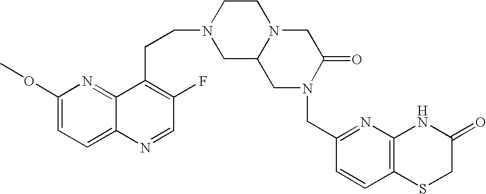 | 6-[(8-{2-[3-fluoro-6-(methyloxy)-1,5- naphthyridin-4-yl]ethyl}-3-oxooctahydro- 2H-pyrazino[1,2-a]pyrazin-2-yl)methyl]- 2H-pyrido[3,2-b][1,4]thiazin-3(4H)-one |
| 29 | 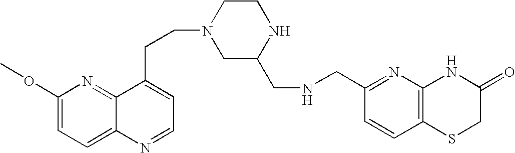 | 6-({[(4-{2-[6-(methyloxy)-1,5- naphthyridin-4-yl]ethyl}-2- piperazinyl)methyl]amino}methyl)-2H- pyrido[3,2-b][1,4]thiazin-3(4H)-one |
| 30 | 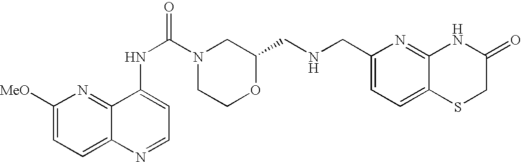 | (2S)-N-[6-(methyloxy)-1,5-naphthyridin- 4-yl]-2-({[(3-oxo-3,4-dihydro-2H- pyrido[3,2-b][1,4]thiazin-6- yl)methyl]amino}methyl)-4- morpholinecarboxamide |

### EXAMPLE 31

### Antimicrobial Activity Assay

Whole-cell antimicrobial activity was determined by broth microdilution using the National Committee for Clinical Laboratory Standards (NCCLS) recommended procedure, Document M7-A6, “Methods for Dilution Susceptibility Tests for Bacteria that Grow Aerobically”. The compounds were tested in serial two-fold dilutions ranging from 0.016 to 16 mcg/mL.

Compounds were evaluated against a panel of Gram-positive organisms, including Staphylococcus aureus, Streptococcus pneumoniae, Streptococcus pyogenes, Enterococcus faecalis and Enterococcus faecium.

In addition, compounds were evaluated against a panel of Gram-negative strains including Haemophilus influenzae, Moraxella catarrhalis, Escherichia coli, Pseudomonas aeruginosa, Proteus mirabilis, Legionella pneumophila, Enterobacter cloacae, Enterobacter aerogenes, Klebsiella pneumoniae and Stenotrophomonas maltophilia.

The minimum inhibitory concentration (MIC) was determined as the lowest concentration of compound that inhibited visible growth. A mirror reader was used to assist in determining the MIC endpoint.

One skilled in the art would consider any compound with a MIC of less than 20 mg/mL to be a potential lead compound. For instance, each of the listed Examples (1 to 30), as identified in the present application, had a MIC≦20 mg/ml against at least one of the organisms listed above.

### EXAMPLE 32

### Rat Infection Model

Certain compounds of this invention were tested in the rat infection model. Specific pathogen-free male Sprague-Dawley CD rats were used for all bacterial strains. Each therapy group consists of 5 animals. Infection was carried out by intrabronchial instillation of 100 ml bacterial suspension for H. influenzae H128, and 50 ml of bacterial suspension for S. pneumoniae 1629 via non-surgical intubation. All compounds were administered at 1, 7, 24 and 31 hour post infection via oral gavage. In each experiment, an additional group of animals was included and served as untreated infected controls. Approximately 17 hour after the end of therapy, the animals were killed and their lungs excised and enumeration of the viable bacteria was conducted by standard methods. The lower limit of detection was 1.7 log 10 CFU/lungs.

In vivo, activity was observed in infection models in rats versus S. pneumoniae 1629 at doses ranging from 25-100 mg/Kg with oral dosing and for some compounds versus H. influenzae H128 at doses from 25-100 mg/Kg with oral dosing. Certain formula (I) compounds showed a greater than 2 log drop in viable counts in the lungs compared to non-treated controls versus S. pneumoniae 1629. Certain compounds of formula (I) showed greater than a 4 log drop in viable counts in the lungs compared to non-treated controls versus H. influenzae H 128. The compounds of this invention are particularly interesting due to their low toxicity with no toxicity being observed in rats with dosing twice daily for 2 days at 50 mg/Kg.

It is to be understood that the invention is not limited to the embodiments illustrated hereinabove and the right is reserved to the illustrated embodiments and all modifications coming within the scope of the following claims.

1. A compound of formula (I) 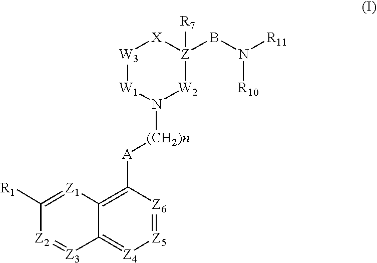
wherein:
Z₁, Z₃, and Z are independently N or CR^{1a};
Z₂, Z₅ and Z₆ are each CR^{1a};
R₁ and R^{1a} are independently at each occurrence hydrogen; cyano; halogen;
hydroxy; (C₁₋₆)alkoxy unsubstituted or substituted by (C₁₋₆)alkoxy, hydroxy, amino, piperidyl, guanidino or amidino any of which is unsubstituted or N-substituted by one or two (C₁₋₆)alkyl, acyl, (C₁₋₆)alkylsulphonyl, CONH₂, hydroxy, (C₁₋₆)alkylthio, heterocyclylthio, heterocyclyloxy, arylthio, aryloxy, acylthio, acyloxy or (C₁₋₆)alkylsulphonyloxy; (C₁₋₆)alkyl; (C₁₋₆)alkylthio; trifluoromethyl; trifluoromethoxy; nitro; azido; acyl; acyloxy; acylthio; (C₁₋₆)alkylsulphonyl; (C₁₋₆)alkylsulphoxide; arylsulphonyl; arylsulphoxide; or an amino, piperidyl, guanidino or amidino group unsubstituted or N-substituted by one or two (C₁₋₆)alkyl, acyl or (C₁₋₆)alkylsulphonyl groups; or R₁ and R^{1a} of Z₂ together form ethylenedioxy;
A is CR₂R₃ or NR^{1b}(C═O);
R₂ is hydrogen; halogen; hydroxy; acyloxy; or (C₁₋₆)alkoxy;
R₃ is hydrogen;
n is independently at each occurrence 0, 1, or 2;
R^{1b} is hydrogen; trifluoromethyl; (C₁₋₆)alkyl; (C₂₋₆)alkenyl; (C₁₋₆)alkoxycarbonyl;
(C₁₋₆)alkylcarbonyl; (C₂₋₆)alkenyloxycarbonyl; aryl; aralkyl; (C₃₋₈)cycloalkyl; heteroaryl;
heteroarylalkyl; or heterocyclyl;
W₁, W₂ and W₃ are CR₄R₅;
R₄, R₈, and R₉ are independently at each occurrence hydrogen; thiol; (C₁₋₆)alkylthio; halogen; trifluoromethyl; azido; (C₁₋₈)alkyl; (C₂₋₆)alkenyl; (C₁₋₆)alkoxycarbonyl; (C₁₋₆)alkylcarbonyl; (C₂₋₆)alkenylcarbonyl; (C₂₋₆)alkenyloxycarbonyl; aryl; aralkyl; aryl; heteroarylalkyl; heteroaryl; heterocyclyl; hydroxy; amino; NR^{1c}R^{1c′}; (C₁₋₆)alkylsulphonyl; (C₂₋₆)alkenylsulphonyl; or (C₁₋₆)aminosulphonyl wherein the amino group is optionally and independently substituted with hydrogen; (C₁₋₆)alkyl; (C₂₋₆)alkenyl; or aralkyl;
R₅ is independently at each occurrence hydrogen or (C₁₋₆)alkyl;
X is O, CR₄R₅, or NR₆;
R₆ is hydrogen, (C₁₋₆)alkyl or together with R₁₀ forms Y;
Y is CR₄R₅CH₂; CH₂CR₄R₅; (C═O); CR₄R₅; CR₄R₅(C═O); or (C═O)CR₄R₅;
R₇ is hydrogen; halogen; hydroxy; or (C₁₋₆)alkyl;
Z is carbon;
B is CR₈R₉ or (C═O);
R₁₀ is hydrogen; (C₁₋₆)alkyl or together with R₆ forms Y;
R₁₁ is UR₁₂;
U is CR₄R₅; C(═O); or S(O)ₙ;
R₁₂ is a substituted or unsubstituted bicyclic carbocyclic or heterocyclic ring system (A): 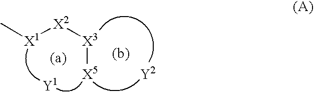
containing up to four heteroatoms in each ring in which at least one of rings (a) and (b) is aromatic;
X¹ is C or N when part of an aromatic ring or CR₁₃ when part of a non aromatic ring;
X² is N, NR₁₄, O, S(O)ₙ, CO or CR₁₃ when part of an aromatic or non-aromatic ring or may in addition be CR₁₅R₁₆ when part of a non aromatic ring;
X³ and X⁵ are independently N or C;
Y¹ is a 0 to 4 atom linker group each atom of which is independently selected from N, NR₁₄, O, S(O)ₙ, CO and CR₁₃ when part of an aromatic or non-aromatic ring or may additionally be CR₁₅R₁₆ when part of a non aromatic ring,
Y² is a 2 to 6 atom linker group, each atom of Y² being independently selected from N, NR₁₄, O, S(O)ₙ, CO and CR₁₃ when part of an aromatic or non-aromatic ring or may additionally be CR₁₅R₁₆ when part of a non aromatic ring;
R₁₃, R₁₅ and R₁₀ are at each occurrence independently selected from: H; (C₁₋₄)alkylthio; halo; (C₁₋₄)alkyl; (C₂₋₄)alkenyl; hydroxy; hydroxy(C₁₋₄)alkyl;
mercapto(C₁₋₄)alkyl; (C₁₋₄)alkoxy; trifluoromethoxy; nitro; cyano; carboxy; amino or aminocarbonyl unsubstituted or substituted by (C₁₋₄)alkyl;
R₁₃ is at each occurrence independently hydrogen; trifluoromethyl; (C₁₋₄)alkyl unsubstituted or substituted by hydroxy, carboxy, (C₁₋₄)alkoxy, (C₁₋₆)alkylthio, halo or trifluoromethyl; (C₂₋₄)alkenyl; or aminocarbonyl wherein the amino group is optionally substituted with (C₁₋₄)alkyl; or
a pharmaceutically acceptable salt thereof;
provided that when Z₁ and Z₃ are CR^{1a}; Z₄ is N; X is O or CR₄R₅; A is CR₂R₃; then R₂ is not hydroxy.

2. A compound according to claim 1, wherein Z₁ and Z₄ are N and Z₃ is CR^{1a}.

3. A compound according to claim 1, wherein Z₁ and Z₃ are CR^{1a} and Z₄ is N.

4. A compound according to claim 1, wherein R₁ is OCH₃.

5. A compound according to claim 1, wherein R^{1a} is at each occurrence independently hydrogen, halogen or cyano.

6. (canceled)

7. A compound according to claim 1, wherein A is CH₂ and n of (CH₂)ₙ is 1.

8. A compound according to claim 1, wherein X is O.

9. A compound according to claim 1, wherein X is CR₄R₅.

10. A compound according to claim 1, wherein X is NR₆.

11. A compound according to claim 10, wherein R₆ and R₇ together form Y.

12. A compound according to claim 11, wherein Y is CR₄R₅(C═O), (C═O) or (C═O)CR₅.

13. A compound according to claim 12, wherein Y is CH₂(C═O), (C═O) or (C═O)CH₂.

14. (canceled)

15. A compound according to claim 1, wherein U is CH₂.

16. A compound according to claim 1, wherein U is SO₂.

17. A compound according to claim 1, wherein U is (C═O).

18. A compound according to claim 1, wherein R₁₂ is:
4H-Pyrido[3,2-b][1,4]thiazin-3-oxo-6-yl;
8-Cyano-2,3-dihydro-benzo[1,4]dioxin-6-yl;
5-Cyano-2,3-dihydro-benzo[1,4]dioxin-7-yl;
4H-Pyrido[3,2-b][1,4]oxazin-3-oxo-6-yl;
8-Fluoro-4H-[1,4]-benzoxazin-3-oxo-6-yl;
4H-Benzo[1,4]thiazin-3-oxo-6-yl;
7-Chloro-4H-pyrido[3,2-b]oxazin-3-oxo-6-yl;
2,3-Dihydro-benzofuran-7-carbonitrile-5-yl; or
[1,3]Oxathiolo[5,4-c]pyridin-6-yl.

19-33. (canceled)

34. A compound according to claim 1, wherein the compound is:
a) 6-({[(1-{2-[6-(methyloxy)-1,5-naphthyridin-4-yl]ethyl}-3-piperidinyl)methyl]amino}methyl)-2H-pyrido[3,2-b][1,4]thiazin-3(4H)-one;
b) N-[(1-{2-[6-(methyloxy)-1,5-naphthyridin-4-yl]ethyl}-3-piperidinyl)methyl]-3-oxo-3,4-dihydro-2H-1,4-benzothiazine-6-sulfonamide;
c) N-[(1-{2-[6-(methyloxy)-1,5-naphthyridin-4-yl]ethyl}-3-piperidinyl)methyl]-3-oxo-3,4-dihydro-2H-pyrido[3,2-b][1,4]thiazine-6-carboxamide;
d) 6-({[(4-{2-[6-(methyloxy)-1,5-naphthyridin-4-yl]ethyl}-2-morpholinyl)methyl]amino}methyl)-2H-pyrido[3,2-b][1,4]thiazin-3(4H)-one;
e) N-[(4-{2-[6-(methyloxy)-1,5-naphthyridin-4-yl]ethyl}-2-morpholinyl)methyl]-3-oxo-3,4-dihydro-2H-pyrido[3,2-b][1,4]thiazine-6-carboxamide;
f) N-[(4-{2-[6-(methyloxy)-1,5-naphthyridin-4-yl]ethyl}-2-morpholinyl)methyl]-3-oxo-3,4-dihydro-2H-1,4-benzothiazine-6-sulfonamide;
g) 6-({[((2R)-4-{2-[6-(methyloxy)-1,5-naphthyridin-4-yl]ethyl}-2-morpholinyl)methyl]amino}methyl)-2H-pyrido[3,2-b][1,4]thiazin-3(4H)-one;
h) 6-({[((2S)-4-{2-[6-(methyloxy)-1,5-naphthyridin-4-yl]ethyl}-2-morpholinyl)methyl]amino}methyl)-2H-pyrido[3,2-b][1,4]thiazin-3(4H)-one;
i) 6-({[((2R)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one;
j) 6-({[((2S)-4-{(2R)-2-hydroxy-2-[6-(methyloxy)-1,5-naphthyridin-4-yl]ethyl}-2-morpholinyl)methyl]amino}methyl)-2H-pyrido[3,2-b][1,4]thiazin-3(4H)-one;
k) N-methyl-4-{2-[6-(methyloxy)-1,5-naphthyridin-4-yl]ethyl}-N-[(3-oxo-3,4-dihydro-2H-pyrido[3,2-b][1,4]thiazin-6-yl)methyl]-2-morpholinecarboxamide;
l) 6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]thiazin-3(4H)-one;
m) 6-({[(4-{2-[3-fluoro-6-(methyloxy)-1,5-naphthyridin-4-yl]ethyl}-2-piperazinyl)methyl]amino}methyl)-2H-pyrido[3,2-b][1,4]thiazin-3(4H)-one;
n) 6-{[7-{2-[3-fluoro-6-(methyloxy)-1,5-naphthyridin-4-yl]ethyl}-3-oxohexahydroimidazo[1,5-a]pyrazin-2(3H)-yl]methyl}-2H-pyrido[3,2-b][1,4]thiazin-3(4H)-one;
o) 6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]oxazin-3(4H)-one;
p) 6-(methyloxy)-4-{2-[(2S)-2-({[(3-oxo-3,4-dihydro-2H-pyrido[3,2-b][1,4]oxazin-6-yl)methyl]amino}methyl)-4-morpholinyl]ethyl}-1,5-naphthyridine-3-carbonitrile;
q) 6-(methyloxy)-4-{2-[(2S)-2-({[(3-oxo-3,4-dihydro-2H-pyrido[3,2-b][1,4]thiazin-6-yl)methyl]amino}methyl)-4-morpholinyl]ethyl}-1,5-naphthyridine-3-carbonitrile;
r) 6-({[((2S)-4-{2-[3-fluoro-6-(methyloxy)-4-quinolinyl]ethyl}-2-morpholinyl)methyl]amino}methyl)-2H-pyrido[3,2-b][1,4]thiazin-3(4H)-one;
s) 6-({[((2S)-4-{2-[3-fluoro-6-(methyloxy)-4-quinolinyl]ethyl}-2-morpholinyl)methyl]amino}methyl)-2H-pyrido[3,2-b][1,4]oxazin-3(4H)-one;
t) 8-fluoro-6-({[((2S)-4-{2-[3-fluoro-6-(methyloxy)-1,5-naphthyridin-4-yl]ethyl}-2-morpholinyl)methyl]amino}methyl)-2H-1,4-benzoxazin-3(4H)-one;
u) 7-chloro-6-({[((2S)-4-{2-[3-fluoro-6-(methyloxy)-1,5-naphthyridin-4-yl]ethyl}-2-morpholinyl)methyl]amino}methyl)-2H-pyrido[3,2-b][1,4]oxazin-3(4H)-one;
v)[((2S)-4-{2-[3-fluoro-6-(methyloxy)-1,5-naphthyridin-4-yl]ethyl}-2-morpholinyl)methyl]([1,3]oxathiolo[5,4-c]pyridin-6-ylmethyl)amine;
w) 7-({[((2S)-4-{2-[3-fluoro-6-(methyloxy)-1,5-naphthyridin-4-yl]ethyl}-2-morpholinyl)methyl]amino}methyl)-2,3-dihydro-1,4-benzodioxin-5-carbonitrile;
x) 5-({[((2S)-4-{2-[3-fluoro-6-(methyloxy)-1,5-naphthyridin-4-yl]ethyl}-2-morpholinyl)methyl]amino}methyl)-2,3-dihydro-1-benzofuran-7-carbonitrile;
y) 6-({[((2S)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one
z) 6-({[((2S)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one;
aa) 6-({[((2R)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one;
ab) 6-({[((2R)-4-{2-[3-fluoro-6-(methyloxy)-1,5-naphthyridin-4-yl]-2-hydroxyethyl}-2-morpholinyl)methyl]amino}methyl)-2H-pyrido[3,2-b][1,4]thiazin-3(4H)-one;
ac) 6-[(8-{2-[3-fluoro-6-(methyloxy)-1,5-naphthyridin-4-yl]ethyl}-4-oxooctahydro-2H-pyrazino[1,2-a]pyrazin-2-yl)methyl]-2H-pyrido[3,2-b][1,4]thiazin-3(4H)-one;
ad) 6-[(8-{2-[3-fluoro-6-(methyloxy)-1,5-naphthyridin-4-yl]ethyl}-3-oxooctahydro-2H-pyrazino[1,2-a]pyrazin-2-yl)methyl]-2H-pyrido[3,2-b][1,4]thiazin-3(4H)-one;
ae) 6-({[(4-{2-[6-(methyloxy)-1,5-naphthyridin-4-yl]ethyl}-2-piperazinyl)methyl]amino}methyl)-2H-pyrido[3,2-b][1,4]thiazin-3(4H)-one; or
af) (2S)—N-[6-(methyloxy)-1,5-naphthyridin-4-yl]-2-({[(3-oxo-3,4-dihydro-2H-pyrido[3,2-b][1,4]thiazin-6-yl)methyl]amino}methyl)-4-morpholinecarboxamide; or
a pharmaceutically acceptable salt thereof.

35. A pharmaceutical composition comprising a compound according to claim 1, or a pharmaceutically acceptable salt thereof, and a pharmaceutically acceptable carrier.

36. A method of treating bacterial infections in mammals which comprises administering to a mammal in need thereof an effective amount of a compound according to claim 1, or a pharmaceutically acceptable salt thereof.
